(19) United States
(12) Patent Application Publication
(10) Pub. No.: US 2025/0369136 A1
(43) Pub. Date: Dec. 4, 2025

(54) PEPTIDE SELF-ASSEMBLY AS A STRATEGY FOR FACILE IMMOBILIZATION OF ENZYMES AND MICROORGANISMS ON ELECTRODES

(71) Applicant: Ramot at Tel-Aviv University Ltd., Tel-Aviv (IL)

(72) Inventors: Lihi ADLER-ABRAMOVICH, Tel-Aviv (IL); Oren BEN-ZVI, Tel-Aviv (IL); Itzhak GRINBERG, Tel-Aviv (IL); Iftach YACOBY, Tel-Aviv (IL); Maxim SOKOL, Tel-Aviv (IL)

(73) Assignee: Ramot at Tel-Aviv University Ltd., Tel-Aviv (IL)

(21) Appl. No.: 19/298,307

(22) Filed: Aug. 13, 2025

Related U.S. Application Data
(63) Continuation of application No. PCT/IL2024/050168, filed on Feb. 13, 2024.
(60) Provisional application No. 63/484,610, filed on Feb. 13, 2023.

(51) Int. Cl. C25B 11/095; C12P 3/00; C25B 1/04; C25B 1/27; C25B 1/50; C25B 11/052; C25B 11/056; H01M 8/0656

(52) U.S. Cl. CPC C25B 11/095; C12P 3/00; C25B 1/04; C25B 1/27; C25B 1/50; C25B 11/052; C25B 11/056; H01M 8/0656

(57) ABSTRACT

A modified fibrous electrode having associated therewith a self-assembled structure formed of a plurality of short aromatic peptides and a biocatalyst associated with the self-assembled structure, electrochemical cells and systems assembled with such modified electrodes and uses thereof are provided.

### RELATED APPLICATIONS

This application is a Continuation of PCT Patent Application No. PCT/IL2024/050168 having International filing date of Feb. 13, 2024 which claims the benefit of priority under 35 USC § 119(e) of U.S. Provisional Patent Application No. 63/484,610, filed on Feb. 13, 2023.

The contents of the above applications are all incorporated by reference as if fully set forth herein in their entirety.

### FIELD AND BACKGROUND OF THE INVENTION

The present invention, in some embodiments thereof, relates to energy conversion, and more particularly, but not exclusively, to employing peptide-based self-assembled structures in facilitating electrochemical reactions performed in the presence a biocatalyst, to electrochemical components and systems comprising such structures and to uses thereof.

Molecular hydrogen (H₂) is a valuable commodity for both the chemical industry and energy market. However, H₂ is not naturally abundant and has to be produced. Most commonly, fossil fuels are used to create “gray” H₂, which is considered unsustainable in the carbon neutral future. The carbon neutral “green” H₂ is currently produced by water electrolysis, powered by renewable energy sources such as solar and wind. While water electrolysis technologies greatly progressed in recent years, this process still faces inherent limitations.

A different approach for H₂ production is the utilization of a biocatalyst. Enzymes possess a variety of advantages as biocatalysts, including negligible overpotential, high specificity, and complete biodegradability (Cracknell et al., Chem. Rev., 2008, 108, 2439-2461). Enzymes can catalyze chemical reactions in a high-yield, scalable cost-efficient manner, and under mild conditions. Hydrogenases, H₂-producing enzymes, are a diverse group of metalloenzymes that catalyze both the reduction of protons into H₂ and the reverse reaction (Lubitz et al., Chem. Rev., 2014, 114, 4081-4148). The direction of catalysis is influenced by the metallic co-factor at the enzyme catalytic site, where [FeFe] hydrogenases are typically more suitable for H₂ production, while [NiFe] hydrogenases tend to favor H₂ oxidation (Fourmond et al., Chem. Commun., 2013, 49, 6840-6842). The reduction of protons to H₂ at the [FeFe] hydrogenase active site requires redox potentials ranging between −0.37 V to −0.45 V under physiological conditions, depending on the specific hydrogenase species and the pH (Rodríguez-Maciá et al., ACS Catal., 2020, 10, 13084-13095). Since these potentials are significantly lower than the 1.23 V required for water splitting, hydrogenases are considered promising biocatalysts. In addition, the active site of hydrogenases is situated near the enzyme surface, and is therefore suitable for direct electron transfer (Morra et al., Bioelectrochemistry, 2015, 106, 258-262; McDonald et al., Nano Lett., 2007, 7, 3528-3534, and Kihara et al., Int. J. Hydrogen Energy, 2011, 36, 7523-7529), that is, a direct electronic communication between the active site and the electrode surface. For this communication to occur, enzymes must be tethered onto a conductive surface in the correct orientation to permit direct electron transfer between the surface and the biomolecule.

While significant progress has been made in this field (see, e.g., Yates et al., Chem. -A Eur. J., 2018, 24, 12164-12182), direct electron transfer is limited by the 2D planar nature of the electrodes, namely, the total amount of enzyme, and therefore the overall activity is limited by the electrode surface area.

Enzymes can be powered by mediated electron transfer, in which the electrons are shuttled to an unbound enzyme by an electron transfer mediator. In the case of hydrogenases, methyl viologen (MV), a common organic dye with a redox potential of −0.44V, is often used for this purpose (Michaelis et al., J. Gen. Physiol., 1933, 16, 859-873; Tatsumi et al., Anal. Chem., 1999, 71, 1753-1759; Lojou, et al., J. Electroanal. Chem., 2005, 579, 199-213). In its simplest form, the mediated electron transfer technique is applied in the bulk volume of the electrochemical cell, although typically inefficiently (Cadoux et al., ChemElectroChem, 2020, 7, 1974-1986). Another approach employs casting of the enzyme and mediator on an electrode surface. The mediator can either be freely diffused or covalently linked to a polymer (redox polymer), in which the enzyme is embedded. Such redox polymers were successfully demonstrated to activate hydrogenases by using MV (Plumeré et al., Nat. Chem., 2014, 6, 822-827) and cobaltocene mediators (Ruth et al., Chem. -A Eur. J., 2020, 26, 7323-7329). Although this method presumably removes the surface limitation, it is currently limited by the small volume and the planar conductive surface. Increasing the thickness of materials cast on the electrode adds distance between the conductive surface and the catalyst, and is therefore hindered by limited diffusion of the mediator (Castañeda-Losada et al., Angew. Chemie—Int. Ed., 2021, 60, 21056-21061; Li et al., J. Am. Chem. Soc., 2019, 141, 16734-16742).

The surface limitation of planar electrodes in both direct and mediated electron transfer settings has prompted researchers to study ways to increase the available surface area. This is usually achieved by the fabrication of an electrode with a 3D architecture, which allows loading of more active material, compared to planar electrodes. Such electrodes can be made of an inorganic compound as was demonstrated with a hierarchically-structured indium-tin oxide electrode (Mersch et al., J. Am. Chem. Soc., 2015, 137, 8541-8549, and Chen et al., Nat. Mater., 2022, 21, 811-818), or carbon-based materials (Sun et al., Nat. Rev. Mater., 2019, 4, 45-60).

Hydrogenases were successfully bound to pyrolytic graphite (Healy et al., Electrochim. Acta, 2011, 56 10786-10790) and carbon nanotubes (Baur et al., Biotechnol. Biotechnol. Equip., 2015, 29, 205-220) to fabricate 3D-hydrogenase electrodes for direct electron transfer. A mediated electron transfer approach which makes use of a 3D-enzymatic electrode requires immobilization of the enzyme in proximity to the electrode (Cadoux et al., ChemElectroChem, 2020, 7, 1974-1986). Such immobilization prevents diffusion of enzyme molecules away from the electrode, and the subsequent significant reduction in its efficiency.

Immobilization on an electrode may be achieved by non-covalent methods such as physical entrapment, physical adsorption and encapsulation in polymer-based matrices (Mohamad et al., Biotechnol. Biotechnol. Equip., 2015, 29, 205-220; Schlager et al., J. Mater. Chem. A, 2017, 5, 2429-2443). In an exemplary set-up, vinylpyrrolidone was used to entrap [NiFe] hydrogenase in a carbon felt electrode (Shiraiwa et al., Bioelectrochemistry, 2018, 123, 156-161).

However, embedding enzymes in polymer matrices could be a tedious process, which might require chemical modifications, voltage application, washing and drying steps, all of which may have a negative effect on the enzyme activity, thus requiring careful planning (Rodriguez-Abetxuko et al., Front. Bioeng. Biotechnol., 2020, 8).

The use of self-assembled peptide-based hydrogels for protein immobilization has been suggested (Seelbach et al., Macromol. Biosci., 2015, 15, 1035-1044).

Peptide-based hydrogels are environmentally friendly, easily synthesized, soft, and biocompatible materials, which mainly consist of aqueous content (Smith et al., Chem. Soc. Rev., 2011, 40, 4563-4577). Supramolecular self-assembly serves as a key approach for the formation of such bulk hydrogels, and low molecular weight hydrogelators have been widely explored (Fichman et al., Acta Biomater., 2014, 10, 1671-1682; Fleming et al, Chem. Soc. Rev., 2014, 43, 8150-8177; Mahler et al., Adv. Mater., 2006, 18, 1365-1370; Jayawarna et al., Adv. Mater., 2006, 18, 611-614; Schnaider et al., Nano Lett., 2020, 20, 1590-1597; Zhang, Interface Focus, 2017, 7). Self-assembly can be triggered by a change in the conditions, i.e., pH (Fleming et al., Chem. Soc. Rev., 2014, 43, 8150-8177) or solvent switch (Fichman et al., Acta Biomater., 2014, 10, 1671-1682), or assisted by enzymatic activity which can facilitate localized self-assembly (Muller et al., Adv. Colloid Interface Sci., 2022, 304; Fores et al., Polymers (Basel), DOI:10.3390/polym13111793).

Fluorenylmethyloxycarbonyl-diphenylalanine (FmocFF) is an aromatic dipeptide building block that can self-assemble in aqueous solutions into nano-scale ordered fibrils, which form a 3D hydrogel network (Mahler et al., Adv. Mater., 2006, 18, 1365-1370; Jayawarna et al., Adv. Mater., 2006, 18, 611-614; Hauser et al., Chem. Soc. Rev., 2010, 39, 2780-2790). Self-assembly of FmocFF is stabilized by π-π interactions between the aromatic rings of the peptide molecules (Dudukovic et al., Langmuir, 2014, 30, 4493-4500; Orbach et al., Biomacromolecules, 2009, 10, 2646-2651; Orbach et al., Langmuir, 2012, 28, 2015-2022; Adler-Abramovich et al., Chem. Soc. Rev., 2014, 43, 6881-6893; Ben-Zvi et al., ACS Nano, 2021, 15, 6530-6539). It was shown that the FmocFF hydrogel stably retains proteins of over 5 kDa while smaller molecules are less restricted (Mahler et al., Adv. Mater., 2006, 18, 1365-1370).

It was demonstrated that [FeFe] hydrogenase can be chemically activated by MV while encapsulated in FmocFF hydrogel (Ben-Zvi et al., ACS Nano, 2021, 15, 6530-6539; PCT/IL2022/050299, published as WO 2022/195592). These documents, however, are silent with regard to the enzyme's activity when subjected to potential application.

Additional background art includes International Patent Application Nos. PCT/IL2006/001174 (published as WO 2007/043048), PCT/IL2011/000435 (published as WO 2011/151832), PCT/IL2018/050773 (published as WO 2019/012545), PCT/IL2019/050788 (published as WO 2020/012490); Adler-Abramovich & Gazit [Chem Soc Rev 2014, 43:6881-6893]; Dudukovic & Zukoski [Langmuir 2014, 30:4493-4500]; Fleming & Ulijn [Chem Soc Rev 2014, 43:8150-8177]; Hauser & Zhang [Chem Soc Rev 2010, 39:2780-2790]; Jayawarna et al. [Adv Mater 2006, 18:611-614]; Jayawarna et al. [Acta Biomater 2009, 5:934-943]; Orbach et al. [Biomacromolecules 2009, 10:2646-2651]; Orbach et al. [Biomacromolecules 2012, 28:2015-2022]; Panda et al. [ACS Appl Mater Interfaces 2010, 2:2839-2848]; RoseFigura et al. [Biochemistry 2011, 50: 1556-1566]; Schnaider et al. [Nano Lett 2020, 20:1590-1597]; Smith et al. [Adv Mater 2008, 20:37-41]; Ulijn & Smith [Chem Soc Rev 2008, 37:664-675]; Widboom et al. [Nature 2007, 447:342-345]; Yang et al. [J Mater Chem 2007, 17:850-854]; Zhang [Interface Focus 2017, 7:20170028]; and Adler-Abramovich. L et al., Carbon Energy, Volume 5, Issue 11, 2023).

### SUMMARY OF THE INVENTION

According to an aspect of some embodiments of the present invention there is provided a composition-of-matter (also referred to herein as a modified electrode) comprising a fibrous electrode having associated therewith a self-assembled structure formed of a plurality of peptides each being of 2 to 6 amino acid residues in length, wherein in each of the peptides, at least one of the amino acid residues is an aromatic amino acid residue, and a biocatalyst, wherein the biocatalyst is associated with the self-assembled structure.

According to some of any of the embodiments described herein, the electrode is a porous fibrous electrode.

According to some of any of the embodiments described herein, the electrode is a fibrous carbon electrode. Alternatively, the electrode is a fibrous, optionally and preferably porous, electrode, made of a material other than carbon, but capable of interacting with the self-assembled structure so as to assure its chemical fixation to the electrode, in addition to a physical fixation.

According to some of any of the embodiments described herein, the electrode is a carbon felt electrode.

According to some of any of the embodiments described herein, the self-assembled structure in entangled with the electrode.

According to some of any of the embodiments described herein, the self-assembled structure is a fibrillar structure.

According to some of any of the embodiments described herein, the self-assembled structure is in a form of a hydrogel.

According to some of any of the embodiments described herein, at least one, or each, peptide in the plurality of peptides comprises an end-capping moiety.

According to some of any of the embodiments described herein, the end-capping moiety is aromatic.

According to some of any of the embodiments described herein, the end-capping moiety is Fmoc.

According to some of any of the embodiments described herein, the end-capping moiety is attached to the N-terminus of the peptide.

According to some of any of the embodiments described herein, at least one, or each, of the aromatic amino acids is phenylalanine.

According to some of any of the embodiments described herein, at least one, or each, of the peptides is a dipeptide.

According to some of any of the embodiments described herein, the dipeptide is or comprises diphenylalanine.

According to some of any of the embodiments described herein, at least one, or each, of the peptides is Fmoc-diphenylalanine.

According to some of any of the embodiments described herein, the composition-of-matter further comprises an electron transfer mediator.

According to some of any of the embodiments described herein, the electron transfer mediator is associated with the self-assembled structure.

According to some of any of the embodiments described herein, the electron transfer mediator is methyl viologen.

According to some of any of the embodiments described herein, the electron transfer mediator is an MXene.

According to some of any of the embodiments described herein, the biocatalyst is an enzyme.

According to some of any of the embodiments described herein, the biocatalyst is an enzyme-producing microorganism.

According to some of any of the embodiments described herein, the biocatalyst catalyzes a redox reaction.

According to some of any of the embodiments described herein, the biocatalyst is an enzyme selected from a hydrogenase, a nitroreductase, a nitrogenase, ferredoxin-NADP+ reductase (FNR) and a Cytochrome P450 enzyme, and any of the other enzymes described herein.

According to some of any of the embodiments described herein, the composition-of-matter further comprises an electrically conducting element electrically connected thereto.

According to an aspect of some embodiments of the present invention there is provided an electrochemical cell comprising, as a working electrode, the composition-of-matter as described herein in any of the respective embodiments and any combination thereof and a power source (an electric power source, to which the composition-of-matter is connectable).

According to some of any of the embodiments described herein, the electrochemical cell further comprises a reference electrode and optionally an auxiliary electrode.

According to some of any of the embodiments described herein, the electrochemical cell comprises two or more working electrodes, at least one, or each, of the working electrodes in the composition-of-matter as described herein.

According to some of any of the embodiments described herein, the electrochemical cell further comprises electrically conducting elements that electrically connect the working electrode or each of the at least two working electrodes, if present, the power source and the reference electrode, if present.

According to some of any of the embodiments described herein, the electrochemical cell further comprises an electrolyte, or otherwise comprises means for introducing thereto an electrolyte.

According to some of any of the embodiments described herein, the electrolyte comprises a substrate of the biocatalyst, for example, it comprises an aqueous solution comprising the substrate, or simply an aqueous solution in case the substrate is water.

According to some of any of the embodiments described herein, the electrochemical cell further comprises a reservoir configured for collecting a gas and/or liquid product formed by reducing or oxidizing the substrate in the presence of the biocatalyst.

According to some of any of the embodiments described herein, the product is a gas usable as fuel in a fuel cell, and the electrochemical cell is forming a part of a system that comprises the fuel cell (e.g., a fuel cell system as described herein).

According to an aspect of some embodiments of the present invention there is provided an electrochemical system comprising the electrochemical cell as described herein in any of the respective embodiments and any combination thereof. The system can comprise, one, two or more electrochemical cells, optionally in combination or in communication (e.g., gaseous communication, liquid communication, electric communication by e.g., electorally conductive elements or wires) with other components, as described herein.

According to an aspect of some embodiments of the present invention there is provided a method of electrically producing a gas and/or liquid formed in a redox reaction catalyzed by a biocatalyst, the method comprising contacting the composition-of-matter as described herein in any of the respective embodiments and any combination thereof, which comprises the biocatalyst, with a substrate of the biocatalyst, and applying potential to the electrode. The contacting can be with an electrochemical cell that comprises the modified electrode (e.g., as a working electrode), as described herein.

According to an aspect of some embodiments of the present invention there is provided a method of performing a redox reaction of a substrate which is catalyzed by a biocatalyst, the method comprising contacting the substrate with the composition-of-matter as described herein in any of the respective embodiments and any combination thereof, which comprises the biocatalyst, and applying potential to the electrode, thereby reducing or oxidizing the substrate. The contacting can be with an electrochemical cell that comprises the modified electrode (e.g., as a working electrode), as described herein.

According to some of any of the embodiments described herein, the biocatalyst comprises or generates a hydrogenase, and the method is for producing H₂.

According to some of any of the embodiments described herein, the substrate is or comprises water.

According to some of any of the embodiments described herein, the biocatalyst comprises or generates a nitrogenase, and the method is for producing ammonia and/or a salt thereof.

Other combination of a biocatalyst and a respective product are contemplated. For example, the biocatalyst can be an oxidase which converts H₂ to water and produces electric energy.

According to an aspect of some embodiments of the present invention there is provided a fuel cell operated by the H₂ produced by a respective method as described herein in any of the respective embodiments. According to some embodiments, the fuel cell and an electrochemical system comprising a modified electrode of the present embodiments are in gaseous communication and together form a fuel cell system. According to an aspect of some embodiments of the present invention there is provided a (e.g., electronic) device or system, operated by a fuel cell system as described herein.

According to an aspect of some embodiments of the present invention there is provided a process of preparing the composition-of-matter as described herein in any of the respective embodiments and any combination thereof, the method comprising contacting the electrode with a solution comprising the plurality of peptides and with a solution comprising the biocatalyst.

According to some of any of the embodiments described herein, the contacting is subsequent to mixing the solutions.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof.

Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 presents a schematic diagram of a process of preparing a composition-of-matter and assembling it in an electrochemical cell, according to exemplary embodiments of the present invention.

FIG. 2 presents the chemical structures of the peptide hydrogelators FmocFF (left and FmocLL (right).

FIGS. 3A-D present cyclic voltammograms obtained using a carbon felt electrode soaked with FmocFF hydrogel supplemented with HydA and MV, at various stages of the electrochemical measurements as shown in FIG. 3E (FIG. 3A), FmocFF hydrogel supplemented with MV (FIG. 3B), FmocFF and active HydA without MV (FIG. 3C), and FmocFF hydrogel only (FIG. 3D), showing the potential (V_{VSH}) versus Current (mA).

FIG. 3E is a schematic illustration showing an electrochemical processes during the cyclovoltammetric measurements shown in FIGS. 3A-D: reduction of MV by the electrode (upon potential application to the electrode) and shuttling of electrons to HydA which catalyzes H₂ production (I); Oxidation of the MV pool at the electrode (II); and H₂ oxidation by HydA which reduces MV, which shuttles the electrons back to the electrode (III). Brighter (yellow) triangles represent MV^{ox}, darker (blue) triangles represent MV^{red}, HydA is represented by its protein crystallographic structure, light (blue) circles represent H₂, dark (gray) area represents the electrode and light (yellow) area represents a FmocFF hydrogel associated with the electrode.

FIGS. 4A-E present a graph showing the accumulated H₂ (in micromoles; μmol) produced overnight by the electrochemical assay versus the amount of HydA enzyme (μg; micrograms) loaded on the working electrode in FmocFF hydrogel (red circles), FmocLL hydrogel (blue triangles), and from solution (black diamonds) (FIG. 4A); and corresponding chronamperometries of overnight electrochemical assays of FmocFF (red), FmocLL (blue), and solution-soaked electrodes (black) for 50 micrograms HydA (FIG. 4B), 100 micrograms HydA (FIG. 4C), 200 micrograms HydA (FIG. 4D), and 400 micrograms HydA (g) (FIG. 4E).

FIGS. 5A-E present overlayed confocal images of fluorescent Cy5-stained HydA enzyme, soaked on carbon felt, (viewed in brightfield, scale bar is 20 micrometers) of HydA in solution (FIG. 5A), HydA encapsulated in FmocLL hydrogel (FIG. 5B) and HydA encapsulated in FmocFF hydrogel (FIG. 5C); a bar graph presenting the residual activity of HydA soaked on carbon felt (%) in either overnight (O·N) aging, immersed in electrolyte solution O·N, or electrochemically activated O·N, for HydA encapsulated in FmocLL hydrogel (red), HydA encapsulated in FmocFF hydrogel (blue), and HydA in solution (gray) (FIG. 5D); and an immunoblot of HydA in electrolyte collected after O·N. electrochemical activation in FmocFF hydrogel, FmocLL hydrogel or solution (FIG. 5E).

FIGS. 6A-G present images of tilted vials of FmocFF at concentrations of 1, 2, 3, 4 and 5 milligrams/mL (FIG. 6A); a graph showing storage modulus (G′) (blue) and loss modulus (G″) (light blue) (in Pa) of FmocFF at concentrations of 1, 2, 3, 4 and 5 milligrams/mL (FIG. 6B); graphs showing storage (solid lines) and loss moduli (dashed lines) kinetics of FmocFF over 35 minutes at concentrations of 3, g/mL (FIG. 6C), 4 mg/mL(FIG. 6D), 5 mg/mL (FIG. 6E) and 6 mg/mL (FIG. 6F), and a bar graph showing the accumulated H₂ (in μmol) after O·N. electrochemical activation of HydA enzyme, encapsulated in 1, 2, 3, 4 and 5 ml/mL FmocFF and soaked on carbon felt electrode (FIG. 6G; error bars represent mean±S.D. of at least six independent experiments).

FIGS. 7A-F present confocal images of fluorescent Cy5-stained HydA enzyme with FmocFF at concentrations of 0 ml/mL (FIG. 7A), 1 ml/mL (FIG. 7B), 2 ml/mL (FIG. 7C), 3 ml/mL (FIG. 7D), 4 ml/mL L (FIG. 7E), and 5 ml/mL (FIG. 7F). Scale bars are 10 micrometers long.

FIGS. 8A-D present scanning electron microscopy images of carbon felt electrode soaked with FmocFF hydrogel (5 mg/mL) at magnifications of ×40 (FIG. 8A), ×450 (FIG. 8B) and ×5500 (FIG. 8C); and a schematic illustration (FIG. 8D) showing an image of H₂-producing working electrode, composed of carbon felt soaked with FmocFF hydrogel encapsulating HydA (left), magnified to show coating of the carbon felt fibers with the self-assembled FmocFF fibrils (middle), and further magnified to show enzyme molecules attached to the FmocFF fibrils and thus immobilized over the carbon fibers (right).

FIGS. 9A-C present a bar graph showing the H₂ (measured in μmol) accumulated from O·N. electrochemical activation of HydA enzyme, encapsulated in FmocFF in the presence of HydA and carbon felt (FmocFFmix) (yellow), in comparison to samples where Hyd A was added to FmocFF which was pre-assembled separately (FmocFFsep) (red), and FmocLL (blue) (FIG. 9A); chronoamperometry graphs measuring current (mA) versus time (hours) of O·N. electrochemical assays of FmocFFmix (yellow), FmocFFsep (purple), and FmocLL (blue) (FIG. 9B); and an immunoblot of HydA in electrolyte collected after O·N. electrochemical assays of FmocFFmix, FmocFFsep, and FmocLL.

FIGS. 10A-F present confocal microscopy images of FmocFF hydrogel with Cy5-labeled FNR (FIG. 10A) and SOD (FIG. 10B) (scale bar is 10 micrometers long); an immunoblot of FNR (FIG. 10C) and SOD (FIG. 10D) collected from electrolyte after O·N. electrochemical assays in FmocFF hydrogel or solution-soaked electrodes (specific FNR or SOD antibodies were used for the respective immunoblots); and cyclic voltammogram graphs of encapsulated FNR (FIG. 10E) and of encapsulated SOD (FIG. 10F).

FIGS. 11A-E present a bar graph showing electrochemical H₂ production (measured in nmol; nanomoles) with 400 micrograms purified enzyme encapsulated in FmocFF hydrogel on carbon felt electrode (white), compared to encapsulated whole E. coli bacteria expressing [FeFe]hydrogenase (gray) (FIG. 11A); a bar graph showing overnight electrochemical H₂ production (measured in nanomoles) of E. coli expressing HydA enzyme, encapsulated in FmocFF (red), FmocLL (blue), and soaked from solution (black) compared to purified clean HydA encapsulated in FmocFF (grey) (FIG. 11B); a bar graph showing the bacterial concentration-dependent (measured in optical density measurements, OD) electrochemical H₂ production (measured in nmol) with encapsulated whole E. coli expressing [FeFe] hydrogenase, at OD levels of 0.4, 0.8, 1.6, 3.2, 6.4 and 12.8 (FIG. 11C); a graph showing the bacterial growth in LB medium at 37° C. post-electrode encapsulation at initial cell concentrations of 0.4 (yellow), 0.8 (green) and 3.2 (blue) cell O.D, over 20 hours (FIG. 11D); and a SEM image (scale bar is 5-micrometer long) of FmocFF hydrogel containing HydA-expressing E. coli bacteria attached to fibers of the carbon felt electrode, with white arrows indicating the position of the bacteria, and an inset demonstrating the bacteria entangled in the FmocFF fibril network (FIG. 11E).

FIG. 12 in a bar graph showing the accumulated H₂ produced upon 20 hours electrochemical activation of carbon felt electrodes having FmocFF gels encapsulating HydA-expressing E. coli and either 2 mM MV or 1, 2 and 3 mg/mL MXene.

FIGS. 13A-B present a bar graph showing the amount of H₂ accumulated from O·N. electrochemical activation of 1 mL, 2 mL and 2×1 mL electrodes containing HydA-expressing whole bacteria, encapsulated in FmocFF gels using MV as electron transfer mediator (FIG. 13A) and comparative plots showing the corresponding chronoamperometries (FIG. 13B).

FIG. 14 presents a schematic illustration of an electrochemical cell according to some embodiments of the present invention

### DESCRIPTION OF SPECIFIC EMBODIMENTS OF THE INVENTION

The present invention, in some embodiments thereof, relates to energy conversion, and more particularly, but not exclusively, to employing peptide-based self-assembled structures in facilitating electrochemical reactions performed in the presence a biocatalyst, to electrochemical components and systems comprising such structures and to uses thereof.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

Using biocatalysts such as enzymes or whole microorganisms in electrochemistry requires their immobilization on electrodes. Such immobilization must resist electrophoresis or the biocatalyst will be lost. Such immobilization should further be sufficiently effective, so as to avoid leakage of the biocatalyst. Harsh chemical treatment is usually required to covalently link catalysts to electrodes, which is harmful for most enzymes and microorganisms.

The present inventors have designed and successfully practiced a novel methodology for successful immobilization of biocatalysts on electrodes, while employing supramolecular self-assembly (self-assembled structures) formed of short peptides. This newly designed methodology is highly resistant to electrophoresis and can be executed under mild conditions which do not affect the biocatalyst activity.

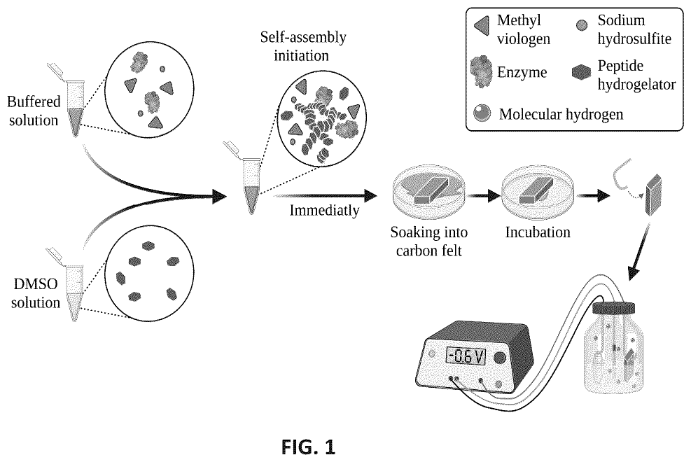

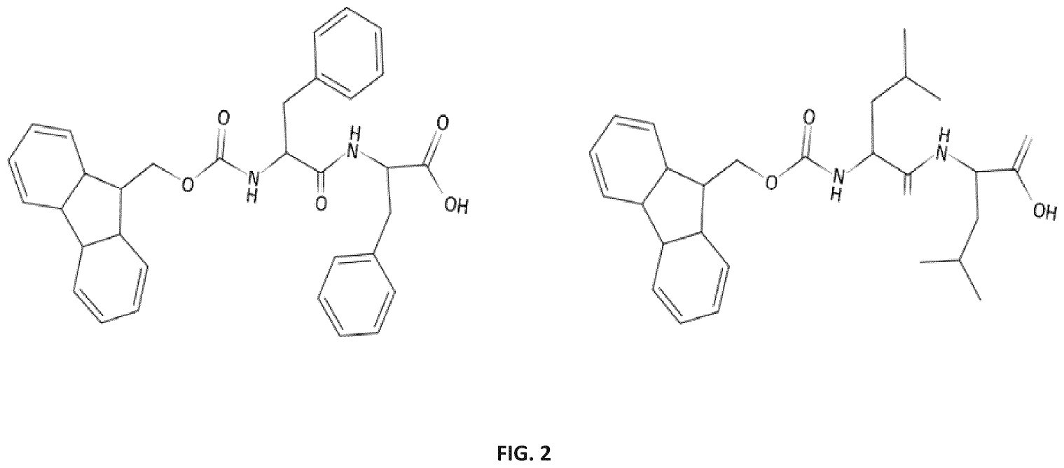

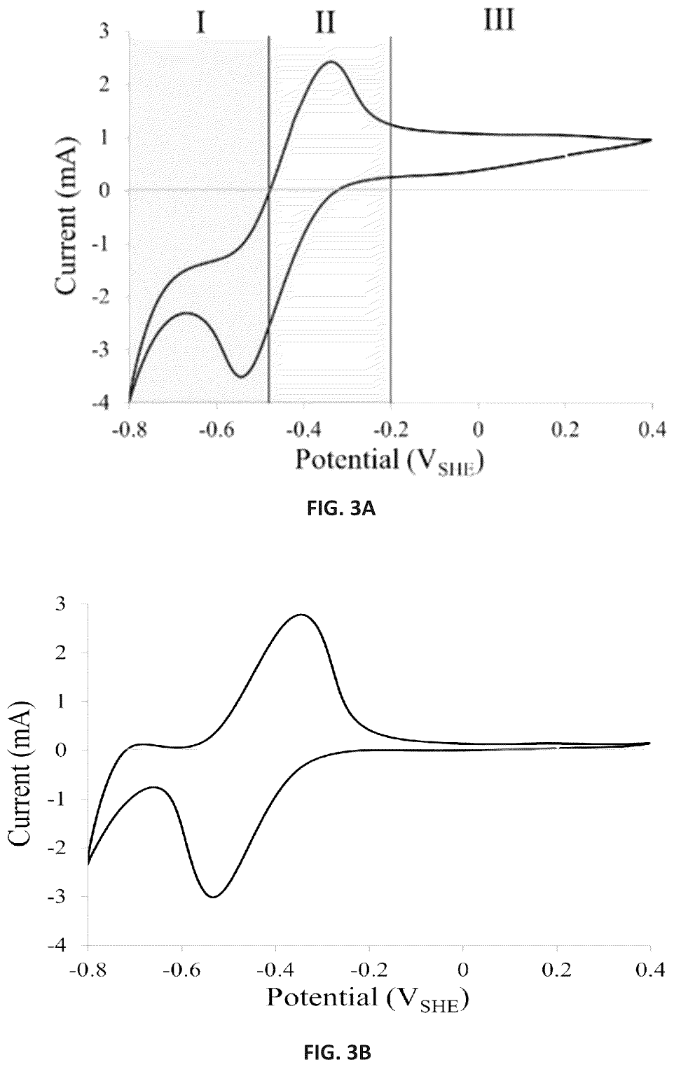

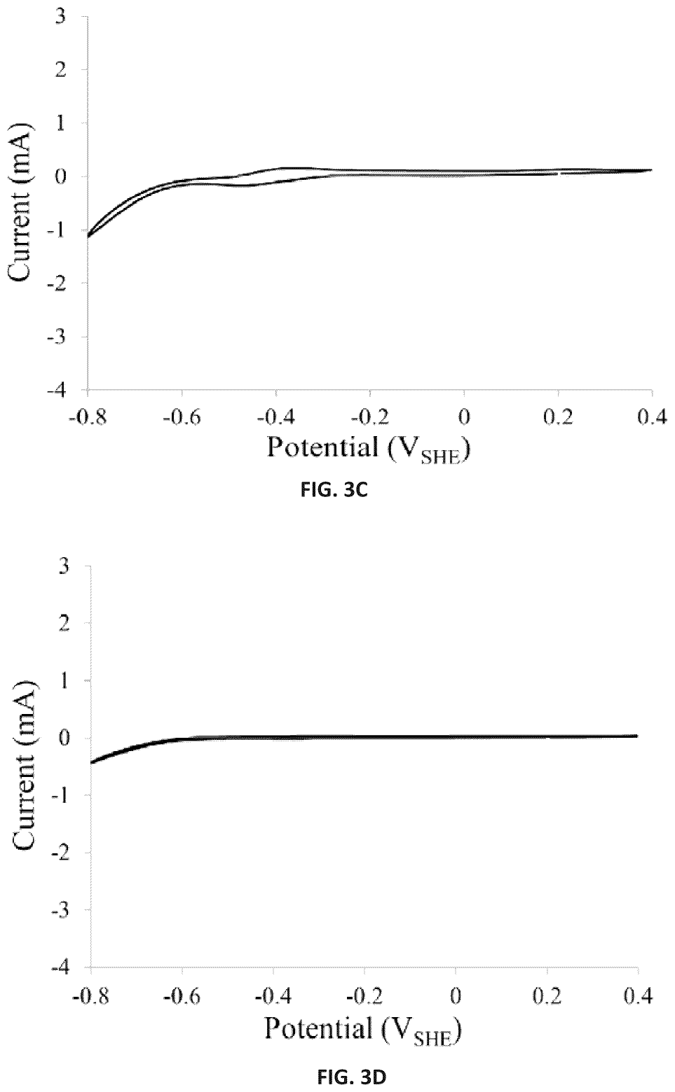

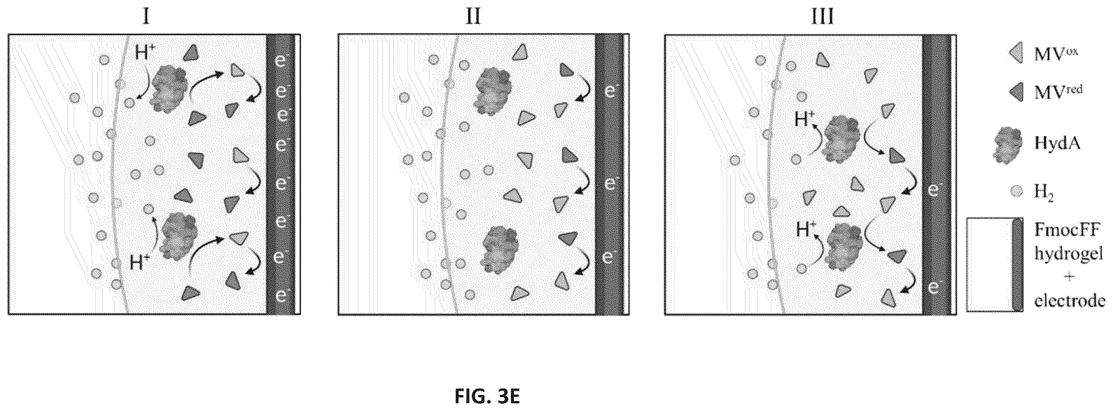

As described in the Examples section that follows, the present inventors have demonstrated that such an enzyme encapsulation approach allows facile and robust immobilization of Chlamydomonas reinhardtii [FeFe] hydrogenase (HydA) on a carbon felt electrode, utilizing the self-assembly of the peptide hydrogelator FmocFF, as schematically exemplified in FIG. 1. The present inventors have demonstrated the high capacity of the FmocFF self-assembled structure to stably retain proteins within the fibrillar structures thereof, while also accumulating over the carbon fibers, thereby serving as a “glue” between the enzyme and the electrode. The overall electrochemical process is mediated by a suitable electron transfer, which is reduced at the carbon fibers and shuttles the electrons to the encapsulated enzyme, to be used for the production of H₂, as schematically exemplified in FIG. 3E. This immobilization approach allows to simultaneously take advantage of porous 3D architecture of the electrode to maintain efficient electron shuttling, overcome the surface limitation to encapsulate proteins at high amounts, under mild conditions, and with relative ease and stability.

The newly designed methodology has been employed to electrochemically produce hydrogen gas at −0.6 V SHE with [FeFe] hydrogenase, both in its pure form and when expressed in whole E. coli bacteria, with high faradaic efficiency.

Some embodiments of the present invention relate to a newly designed method for efficiently encapsulating biocatalysts that promote redox reactions, such as [FeFe] hydrogenase, [NiFe] hydrogenase, Nitrogenase, ferredoxin-NADP+ reductase (FNR) and Cytochrome P450, among others, or microorganisms that produce such enzymes, on an electrode, preferably a porous hydrophobic electrode such as a carbon electrode, for example, a 3D carbon felt electrode, using peptide-based self-assembled structures. Glue-like immobilization of the biocatalyst on, for example, the carbon fibers of a 3D carbon felt electrode, is achieved under mild conditions. High biocatalyst loads, prolonged resistance against electrophoresis, and highly efficient hydrogen production have been demonstrated, indicating its applicability to a wide variety of enzymes and enzyme-containing microorganisms (e.g., bacteria).

Among the applications of the present embodiments are energy conversion to hydrogen and reduction of CO₂ chemical production with redox enzymes.

The electrodes of the present invention assembled with encapsulated enzyme or whole bacteria can be used in devices that convert electrical energy into H₂ gas. Such a device can be coupled with a fuel-cell and/or a gas storage device. The end user can be any organization or homeowner that needs to store energy. Other users can be ammonia production facilities or others that use H₂ gas as a commodity and are interested in green hydrogen. The electrodes of the present embodiments, assembled with encapsulated biocatalyst, can also be used in devices that convert H₂ gas into electrical energy, or in devices that reduce CO₂ chemical production.

Embodiments of the present invention relate to compositions-of-matter in which a self-assembled structure (e.g., supramolecular structure) formed of a plurality of peptides is associated with an electrode and with a biocatalyst that is encapsulated within the self-assembled structure.

Such compositions-of-matter are also referred to herein as modified electrodes or simply as electrodes. Embodiments of the present invention further relate to electrochemical assemblies made of these compositions-of-matter (e.g., electrochemical cells or systems) and to uses thereof, for example, in operating fuel cells.

### Compositions-of-Matter:

According to an aspect of some embodiments of the present invention there is provided a composition-of-matter comprising a fibrous electrode having associated therewith a self-assembled structure made formed of a plurality of peptides each being of 2 to 6 amino acid residues in length, wherein in each of the peptides, at least one of the amino acid residues is an aromatic amino acid residue, and a biocatalyst, wherein the biocatalyst is associated with the self-assembled structure.

According to an aspect of some embodiments of the present invention there is provided a composition-of-matter comprising a carbon electrode, for example, a carbon fibrous and/or porous electrode, having associated therewith a self-assembled structure made formed of a plurality of peptides each being of 2 to 6 amino acid residues in length, wherein in each of the peptides, at least one of the amino acid residues is an aromatic amino acid residue, and a biocatalyst, wherein the biocatalyst is associated with the self-assembled structure.

According to some of any of the embodiments described herein, the composition-of-matter further comprises an electron transfer mediator as described herein in any of the respective embodiments and any combination thereof. According to some of these embodiments, the electron transfer mediator is associated with the self-assembled structure.

A composition-of-matter as described herein in any of the respective embodiments and any combination thereof is also referred to herein as a modified electrode or simply, for brevity, as an electrode.

### Electrode:

According to some of any of the embodiments described herein, the electrode forming the composition-of-matter described herein is a three-dimensional (3D) electrode.

According to some of these embodiments, the electrode features a thickness of at least 1 mm, or at least 2 mm, or at least 3 mm, for example, of 1 to 10 mm, or of 2 to 10 mm, or of 3 to 10 mm, or of 1 to 8 mm, or of 2 to 8 mm, or of 3 to 8 mm, or of 4 to 8 mm, or of 2 to 6 mm, or of 3 to 6 mm, or of 4 to 6 mm, including any intermediate values and subranges therebetween.

Generally, but not necessarily, the electrode (e.g., 3D electrode) features a length (height) and/or width, independently, of from about 1 mm to about 100 cm, or from about 1 mm to about 50 cm, or from about 1 mm to about 30 cm, or from about 1 mm to about 20 cm, or from about 10 mm to about 100 cm, or from about 10 mm to about 50 cm, or from about 10 mm to about 30 cm, or from about 10 cm to about 100 cm, or from about 10 cm to about 50 cm, or from about 10 cm to about 30 cm, or from about 1 mm to about 100 mm, or from about 10 cm to about 200 cm, or from about 10 cm to about 300 cm, or from about 10 cm to about 500 cm, or from about 1 mm to about 100 mm, or from about 1 mm to about 50 mm, or from about 1 mm to about 20 mm, including any intermediate values and subranges therebetween.

Generally, but not necessarily, the 3D electrode features a volume in a range of from 1 mL to 1 L, or from 1 mL to 10 L, or from 1 mL to 5 L, or from 100 mL or 1 L, or from 100 mL to 10 L, or from 100 mL to 5 L, or from 1 mL to 100 mL, or from 10 mL to 100 mL, or from 1 L to 10 L, including any intermediate values and subranges therebetween.

In embodiments where two or more electrodes are assembled into an array, as described herein, the above volume ranges apply to the total volume of the electrodes or to the volume of each electrode alone.

Generally, but not necessarily, the electrode (e.g., 3D electrode) features a surface area that ranges from about 1 to about 10,000 cm², or from about 10 to about 10,000, or from about 100 to about 10000, or from about 1 to about 20000, or from about 10 to about 20000, or from about 100 to about 20000, or from about 10000 to about 20000, or from about 200 to about 20000, or from about 500 to about 15000, cm², or from about 1 to about 5000, or from about 1 to about 2000, or from about 1 to about 1000, cm², including any intermediate values and subranges therebetween.

According to some of these embodiments, the electrode features a thickness of at least 1 mm, or at least 2 mm, or at least 3 mm, for example, of 1 to 10 mm, or of 2 to 10 mm, or of 3 to 10 mm, or of 1 to 8 mm, or of 2 to 8 mm, or of 3 to 8 mm, or of 4 to 8 mm, or of 2 to 6 mm, or of 3 to 6 mm, or of 4 to 6 mm, including any intermediate values and subranges therebetween.

According to some of any of the embodiments described herein, the electrode (e.g., 3D electrode) is a porous electrode. In some embodiments, the electrode features porosity of at least 50%, or at least 60% or at least 70%, or at least 80%, or at least 90%, for example, of from 50 to 99%, or of from 60 to 99%, or of from 70 to 99%, or of from 80 to 99% or of from 90 to 99%, including any intermediate values and subranges therebetween. In some embodiments, the electrode features surface area (for example, as determined in BET measurements) of at least 0.01, or at least 0.05, preferably of at least 0.1, m²/gram, for example, of from 0.01 to 100.4 m²/gram, or from 0.1 to 10 m²/gram, or from 0.1 to 5 m²/gram, or from 0.1 to 1 m²/gram, including any intermediate values and subranges therebetween. According to some of any of the embodiments described herein, the electrode (e.g., 3D electrode) is a fibrous electrode, which comprises in at least a portion thereof a fibrous structure, made of a plurality of fibers, preferably entangled to one another. The average diameter of the fibers can range, for example, from 0.1 micron to 100 microns, or from 0.1 micron to 50 microns, or from 0.1 micron to 20 microns, or from 0.1 micron to 10 microns, or from 1 micron to 100 microns, or from 1 micron to 50 microns, or from 1 micron to 20 microns, or from 1 micron to 10 microns, or from 5 microns to 50 microns, or from 5 microns to 20 microns, or from 5 microns to 15 microns, including any intermediate values and subranges therebetween.

According to some of any of the embodiments described herein, the electrode (e.g., 3D electrode) is a porous fibrous electrode. According to some of these embodiments, the electrode comprises in at least a portion thereof a fibrous structure, made of a plurality of fibers, preferably entangled to one another. The average diameter of the fibers can range, for example, from 0.1 micron to 100 microns, or from 0.1 micron to 50 microns, or from 0.1 micron to 20 microns, or from 0.1 micron to 10 microns, or from 1 micron to 100 microns, or from 1 micron to 50 microns, or from 1 micron to 20 microns, or from 1 micron to 10 microns, or from 5 microns to 50 microns, or from 5 microns to 20 microns, or from 5 microns to 15 microns, including any intermediate values and subranges therebetween; and features porosity of at least 50%, or at least 60% or at least 70%, or at least 80%, or at least 90%, for example, of from 50 to 99%, or of from 60 to 99%, or of from 70 to 99%, or of from 80 to 99% or of from 90 to 99%, including any intermediate values and subranges therebetween. In some embodiments, the electrode features surface area (for example, as determined in BET measurements) of at least 0.01, or at least 0.05, preferably of at least 0.1, m²/gram, for example, of from 0.01 to 100.4 m²/gram, or from 0.1 to 10 m²/gram, or from 0.1 to 5 m²/gram, or from 0.1 to 1 m²/gram, including any intermediate values and subranges therebetween.

According to some of any of the embodiments described herein, the electrode (e.g., 3D electrode) has a density in a range of at least 1 mg/cm³, or of at least 10 mg/cm³, or of at least 50 mg/cm³, for example, of from about 1 to about 1,000, or from about 1 to about 500, or from about 1 to about 200, or from about 10 to about 1,000, or from about 10 to about 500, or from about 10 to about 200, mg/cm³, including any intermediate values and subranges therebetween.

According to some of any of the embodiments described herein, the electrode can be made of any material or combination of materials that provide a suitable conductivity and are inert to electrochemical reaction conditions. Exemplary such materials include carbon, graphite, graphene, noble conductive metals, precious conductive metals, and the like. According to some of any of the embodiments described herein, the electrode is not a platinum (Pt) or a platinum group metal (PGM) electrode, that is, it comprises less than 50%, preferably less than 20% or less than 10%, or less than 5%, or less than 1%, by weight, or null, platinum or a platinum group metal.

As used herein and in the art, the phrase “platinum group metals”, abbreviated PGM, collectively refers to six metallic elements clustered together in the periodic table, which include ruthenium, rhodium, palladium, osmium, iridium, and platinum.

According to some of any of the embodiments described herein, the electrode is made of a material or a combination of material which provide a suitable conductivity, and, in addition, are capable of interacting, chemically, yet non-covalently, with the self-assembled structure. Such materials, in other words, are capable of interacting via, for example, hydrophobic interactions, Van-der-Waals interactions, and/or aromatic interactions, with the self-assembled structure. In some of these embodiments, the electrode is made of an organic material, for example, is a carbon electrode, which comprises at least 50%, or at least 70%, or at least 80%, or at least 90%, or at least 95%, or at least 99%, or 100%, by weight, carbon. Any form of carbon is contemplated.

According to some of any of the embodiments described herein, the electrode (e.g., 3D electrode) is a carbon electrode.

Carbon electrodes can be made of glassy carbon, screen-printed carbon, carbon films, carbon fibers, carbon paste, carbon nanotubes and others.

According to some of any of the embodiments described herein, the electrode (e.g., 3D electrode) is a carbon porous electrode.

According to some of any of the embodiments described herein, the electrode (e.g., 3D electrode) is carbon porous fibrous electrode.

According to some of any of the embodiments described herein, the electrode (e.g., 3D electrode) is a porous carbon fiber electrode.

According to some of any of the embodiments described herein, the electrode (e.g., 3D electrode) is a carbon fiber electrode, featuring porosity and/or density and/or surface area and/or average fiber diameter as described herein in any of the respective embodiments.

According to some of any of the embodiments described herein, the electrode is a carbon fiber electrode, which comprises in at least a portion thereof a fibrous structure, made of a plurality of fibers, preferably entangled to one another. The average diameter of the fibers can range, for example, from 0.1 micron to 100 microns, or from 0.1 micron to 50 microns, or from 0.1 micron to 20 microns, or from 0.1 micron to 10 microns, or from 1 micron to 100 microns, or from 1 micron to 50 microns, or from 1 micron to 20 microns, or from 1 micron to 10 microns, or from 5 microns to 50 microns, or from 5 microns to 20 microns, or from 5 microns to 15 microns, including any intermediate values and subranges therebetween. According to some of any of these embodiments, the carbon electrode features porosity of at least 50%, or at least 60% or at least 70%, or at least 80%, or at least 90%, for example, of from 50 to 99%, or of from 60 to 99%, or of from 70 to 99%, or of from 80 to 99% or of from 90 to 99%, including any intermediate values and subranges therebetween. In some of these embodiments, the electrode features surface area (for example, as determined in BET measurements) of at least 0.01, or at least 0.05, preferably of at least 0.1, m²/gram, for example, of from 0.01 to 100.4 m²/gram, or from 0.1 to 10 m²/gram, or from 0.1 to 5 m²/gram, or from 0.1 to 1 m²/gram, including any intermediate values and subranges therebetween.

According to some of any of the embodiments described herein, the electrode (e.g., 3D electrode) is a carbon felt electrode, and in some embodiments, it is a carbon felt electrode featuring porosity and/or density and/or surface area and/or average fiber diameter as described herein in any of the respective embodiments.

By “carbon felt electrode” it is meant an electrode made of a porous and flexible material composed mainly of carbon fibers.

Exemplary commercially available carbon felt that are usable in the context of the present embodiments include, but are not limited to, electrodes marketed by SGL carbon, such as SIGRACELL® Carbon felt, electrodes marketed FuelCellStore, electrodes marketed by MTI corporation, electrodes marketed by Toray Industries under the trade name Toray Carbon Felt Electrode, electrodes marketed by Fuel Cell Earth, for example, Graphite Felt electrode. Any other commercially available or costume-made carbon electrodes are contemplated.

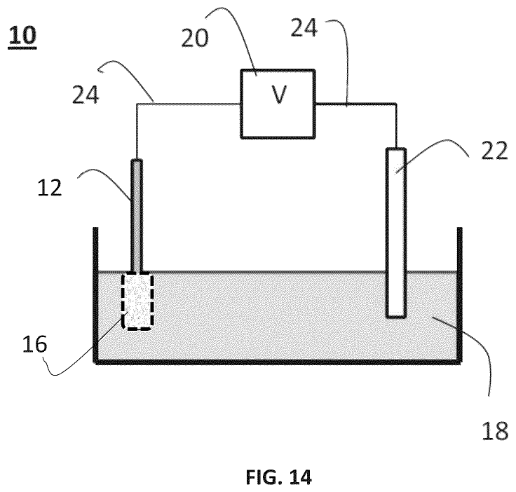

In some embodiments, the electrode is electrically connectable to other parts of an electrochemical system (e.g., electrochemical cell, fuel cell, a combination thereof, etc.) via electrically conducting wires, for example, conducting metal foils such as, but not limited to, Ni foils, Pt foils, etc. In some embodiments, the electrode is in electrical communication with electrically conductive wires. See, for example, FIG. 14.

According to some of any of the embodiments described herein, the composition-of-matter comprises an array of electrodes, that is, two or more electrodes, wherein at least one, at least two and preferably each electrode is independently an electrode as described herein in any of the respective embodiments and any combination thereof. According to some of these embodiments, all the electrodes in the array are the same, that is, are made of the same material (e.g., carbon), and feature the same properties (e.g., fibers' dimension, porosity, density, surface area, volume etc.), as described herein in any of the respective embodiments and any combination thereof. Alternatively, one or more electrodes in the array differ from one another, by one or more of the material composing the electrode and/or the properties of the electrode.

According to some of any of the embodiments described herein, the composition-of-matter comprises an array of electrodes, that is, two or more electrodes, wherein at least one, at least two and preferably each electrode is a carbon felt electrode as described herein in any of the respective embodiments and any combination thereof. In some of these embodiments, each of carbon felt electrodes features fibers' dimensions and porosity as described herein in any of the respective embodiments. In some of these embodiments, the surface area described as described herein, relates to the array of electrodes, that is, it is the sum of the surface area of the two or more electrodes in the array.

### Self-Assembled Structure:

According to some of any of the embodiments described herein a self-assembled structure is associated with the electrode.

Herein, by “associated with” in the context of these embodiments, it is meant that the self-assembled structure is in contact with the electrode, and/or bound to the electrode by chemical interactions, for example, by covalent and/or non-covalent bonding (e.g., hydrogen bonds, van der Waals bonds, electrostatic interaction, and/or hydrophobic interaction and/or aromatic interactions) and/or physical interactions (e.g., by means of absorption, entrapment, entanglement, embedment).

In some of any of the embodiments described herein, the self-assembled structure is associated with the electrode at least by physical interactions, and is some embodiments, it is entangled with the electrode, for example, entangled with the fibers of a fibrous electrode and/or absorbed to the electrode's surface and/or within the electrode's pores (if present). In exemplary embodiments, the self-assembled structure is a fibrous or fibrillar structure as described herein, and fibers of the self-assembled structure are entangled with fibers of a fibrous electrode as described herein.

Herein, the term “self-assembled structure” is also referred to interchangeably as “supramolecular self-assembly”, and is made of a plurality of peptides that self-assemble to form the structure, as described in further detail hereinafter.

The self-assembled structure according to the present embodiments is formed of a plurality of short peptides, each being independently of 2, 3, 4, 5 or 6, or of 2, 3 or 4, or of 2, amino acid residues in length. In each of the peptides, one or more of the amino acid residues is an aromatic amino acid residue, as defined herein. The peptides in the plurality of peptides that form the self-assembled structure can be the same or different, and when different, the peptides may differ from one another by number and/or type of the amino acid residues.

According to some of any of the embodiments described herein, at least 50%, or at least 60%, or at least 70%, or at least 80% or at least 90%, or at least 95%, or at least 98%, or all, of the peptides, are the same peptides in terms of number and type of amino acid residues.

According to some of any of the embodiments described herein, the self-assembled structure is a fibrillar structure (e.g., comprises one or more fibrils). In some embodiments, the structures are self-assembled upon forming aromatic interactions between the aromatic portion of the aromatic molecules that form the structures.

Herein, the term “fibril”, and its adjectival form “fibrillar”, refers to a structure characterized by a narrow cross-section, preferably having an average diameter of less than 10% (optionally less than 1%) of the length of the structure (along a long axis thereof).

According to some of any of the embodiments described herein, the structure comprises a nanostructure (e.g., a fibrillar nanostructure).

As used herein the phrase “nanostructure” refers to a structure having a diameter or a cross-section in at least one dimension thereof of less than 1 m (preferably less than about 100 nm, more preferably less than about 50 nm, and even more preferably less than about 20 nm, e.g., of about 10 nm).

As used herein the phrase “fibrillar nanostructure” refers to a filament or fiber having a diameter or a cross-section width of less than 1 m (preferably less than about 100 nm, more preferably less than about 50 nm, and even more preferably less than about 20 nm, e.g., of about 10 nm). The length of the fibrillar nanostructure (along its long axis) is preferably at least 1 nm, more preferably at least 10 nm, even more preferably at least 100 nm and even more preferably at least 500 nm.

According to some of any of the embodiments described herein, the self-assembled structure is a fibrillar structure, which comprises a plurality of fibrils, preferably associated with one another, as described herein. According to some embodiments, the plurality of peptides self-assemble, via aromatic π-π interactions, to form a plurality of fibrous structures, e.g., fibrils, and these fibrous structures further rearrange for form a fibrillar structure in which the fibrous structures are entangled with one another.

According to some of any of the embodiments described herein, the self-assembled structure forms a 3-dimensional network.

According to some of any of the embodiments described herein, the self-assembled structure is in a form of a hydrogel, for example, a hydrogel comprising an aqueous liquid in addition to the self-assembled structure.

As used herein and in the art, the term “hydrogel” refers to a gel, typically semi-solid, material that comprises 3-dimensional fibrous networks formed of natural or synthetic chains, typically containing more than 80%, or more than 90% or more than 95% or more than 99%, by weight or by volume, water or an aqueous solution.

As used herein the phrase “fibrous network” refers to a set of connections formed between a plurality of fibrous components. Herein, the fibrous components are optionally composed of a plurality of fibrils (e.g., fibrillar nanostructures), at least a portion of which, or each, being formed upon self-assembly of aromatic building blocks (e.g., aromatic amino acid).

According to some of any of the embodiments described herein, the aqueous solution is water.

According to some of any of the embodiments described herein, the aqueous solution is a buffer.

As described herein, the peptides forming the self-assembled structure according to embodiments of the invention include at least one aromatic amino acid residue. According to some of any of the embodiments described herein, in at least a portion, or in all of the plurality of peptides, each of the amino acid residues in the peptide is an aromatic amino acid residue.

By “aromatic amino acid” it is meant an amino acid, or an amino acid residue in a peptide comprising same, that has an aromatic moiety or group, as defined herein, is its side chain. In exemplary embodiments, an aromatic amino acid has, for example, a substituted or unsubstituted naphthalenyl or a substituted or unsubstituted phenyl, in its side chain. The substituted phenyl may be, for example, pentafluorophenyl, iodophenyl, biphenyl and/or nitrophenyl.

As used herein, the phrase “aromatic group” or “aromatic moiety” describes a monocyclic or polycyclic moiety having a completely conjugated pi-electron system. The aromatic group can be an all-carbon moiety or can include one or more heteroatoms such as, for example, nitrogen, sulfur or oxygen. The aromatic group can be substituted or unsubstituted, whereby when substituted, the substituent can be, for example, one or more of alkyl, trihaloalkyl, alkenyl, alkynyl, cycloalkyl, aryl, heteroaryl, heteroalicyclic, halo, nitro, azo, hydroxy, alkoxy, thiohydroxy, thioalkoxy, cyano and amine.

Exemplary aromatic groups include, for example, phenyl, biphenyl, naphthalenyl, phenanthrenyl, anthracenyl, [1,10]phenanthrolinyl, indoles, thiophenes, thiazoles and, [2,2′]bipyridinyl, each being optionally substituted. Thus, representative examples of aromatic groups that can serve as the side chain within the aromatic amino acid described herein include, without limitation, substituted or unsubstituted naphthalenyl, substituted or unsubstituted phenanthrenyl, substituted or unsubstituted anthracenyl, substituted or unsubstituted [1,10]phenanthrolinyl, substituted or unsubstituted [2,2′]bipyridinyl, substituted or unsubstituted biphenyl and substituted or unsubstituted phenyl. The aromatic group can alternatively be substituted or unsubstituted heteroaryl such as, for example, indole, thiophene, imidazole, oxazole, thiazole, pyrazole, pyridine, pyrimidine, quinoline, isoquinoline, quinazoline, quinoxaline, and purine.

In some of any of the embodiments described herein, the aromatic molecule comprises at least one aromatic moiety that is an all-carbon aromatic moiety, e.g., an aryl as defined herein.

In some of any of the embodiments described herein, the aromatic amino acid is phenylalanine. In some such embodiments, at least a portion, or each peptide in the structure comprises a plurality of phenylalanine residues.

In some of any of the embodiments described herein, the peptide is a dipeptide (i.e., having two amino acid residues), and in some embodiments it is a homodipeptide (i.e., having two amino acid residues which are identical with respect to their side-chain). The dipeptide (e.g., homodipeptide) may optionally be modified by an end-capping moiety according to any of the respective embodiments described herein).

Exemplary aromatic homodipeptides include, but are not limited to, phenylalanine-phenylalanine dipeptide (diphenylalanine peptide), naphthylalanine-naphthylalanine dipeptide, phenanthrenylalanine-phenanthrenylalanine dipeptide, anthracenylalanine-anthracenylalanine dipeptide, [1,10]phenanthrolinylalanine-[1,10]phenanthrolinylalanine dipeptide, [2,2′]bipyridinylalanine-[2,2′]bipyridinylalanine dipeptide, (pentahalo-phenylalanine)-(pentahalo-phenylalanine) dipeptide (e.g., (pentafluoro-phenylalanine)-(pentafluoro-phenylalanine) dipeptide), (amino-phenylalanine)-(amino-phenylalanine) dipeptide, (dialkylamino-phenylalanine)-(dialkylamino-phenylalanine) dipeptide, (halophenylalanine)-(halophenylalanine) dipeptide (e.g., (4-fluoro-phenylalanine)-(4-fluoro-phenylalanine) dipeptide), (alkoxy-phenylalanine)-(alkoxy-phenylalanine) dipeptide, (trihalomethyl-phenylalanine)-(trihalomethyl-phenylalanine) dipeptide, (4-phenyl-phenylalanine)-(4-phenyl-phenylalanine) dipeptide and (nitro-phenylalanine)-(nitro-phenylalanine) dipeptide, each of which dipeptides may optionally be modified (e.g., according to any of the respective embodiments described herein). In some of any of embodiments described herein relating to a homodipeptide, the homodipeptide is phenylalanine-phenylalanine and/or naphthylalanine-naphthylalanine dipeptide.

Representative examples of suitable aromatic peptides other than homodipeptides include, without limitation, phenylalanine-glycine (Phe-Gly), phenylalanine-arginine-glycine-aspartic acid (Phe-Arg-Gly-Asp), and arginine-glycine-aspartic acid-phenylalanine (Arg-Gly-Asp-Phe) peptides, each of which peptides may optionally be modified (e.g., according to any of the respective embodiments described herein).

According to some of any of the embodiments described herein, at least a portion, or each peptide in the plurality of peptides (according to any of the respective embodiments described herein) comprises an end-capping moiety.

The phrase “end-capping moiety”, as used herein, refers to a moiety that when attached to the terminus of a peptide, modifies the peptide terminus. The end-capping modification typically results in masking the charge of the peptide terminus, and/or altering chemical features thereof, such as, hydrophobicity, hydrophilicity, reactivity, solubility and the like. Examples of moieties suitable for peptide end-capping modification can be found, for example, in Green et al., “Protective Groups in Organic Chemistry”, (Wiley, 2^{nd} ed. 1991) and Harrison et al., “Compendium of Synthetic Organic Methods”, Vols. 1-8 (John Wiley and Sons, 1971-1996).

Representative examples of N-terminus end-capping moieties include, but are not limited to, formyl, acetyl (also denoted herein as “Ac”), trifluoroacetyl, benzyl, benzyloxycarbonyl (also denoted herein as “Cbz”), tert-butoxycarbonyl (also denoted herein as “Boc”), trimethylsilyl (also denoted “TMS”), 2-trimethylsilyl-ethanesulfonyl (also denoted “SES”), trityl and substituted trityl groups, allyloxycarbonyl, 9-fluorenylmethyloxycarbonyl (also denoted herein as “Fmoc”), and nitro-veratryloxycarbonyl (“NVOC”). Fmoc is an exemplary end-capping moiety.

Representative examples of C-terminus end-capping moieties are typically moieties that lead to acylation of the carboxy group at the C-terminus and include, but are not limited to, benzyl and trityl ethers as well as alkyl ethers, tetrahydropyranyl ethers, trialkylsilyl ethers, allyl ethers, monomethoxytrityl and dimethoxytrityl. Alternatively, the —COOH group of the C-terminus end-capping may be modified to an amide group.

Other end-capping modifications include replacement of the amine and/or carboxyl with a different moiety, such as hydroxyl, thiol, halide, alkyl, aryl, alkoxy, aryloxy and the like, as these terms are defined herein.

In some embodiments of the present invention, a self-assembled structure is made of a plurality of peptides and all of the peptides composing the structure are end-capping modified. In some of these embodiments, the aromatic amino acids are modified only at the N-terminus or the C-terminus thereof, e.g., resulting in a structure that has a negative net charge or a positive net charge, respectively. In another embodiment, the aromatic amino acids are modified at both the N-terminus and the C-terminus thereof, e.g., resulting in an uncharged structure.

According to some of any of the embodiments described herein, the peptide is end-capping modified at the N-terminus thereof.

The end-capping moiety may optionally be aromatic or non-aromatic. According to some of any of the respective embodiments described herein, the end capping moiety is an aromatic end-capping moiety.

According to some of any of the embodiments described herein, the end-capping moiety is attached to the N-terminus of the peptide, and the end-capping moiety is an aromatic moiety (e.g., Fmoc).

Representative examples of aromatic end capping moieties suitable for N-terminus modification include, without limitation, fluorenylmethyloxycarbonyl (Fmoc). Representative examples of aromatic end capping moieties suitable for C-terminus modification include, without limitation, benzyl, benzyloxycarbonyl (Cbz), trityl and substituted trityl groups.

Representative examples of non-aromatic end capping moieties suitable for N-terminus modification include, without limitation, formyl, acetyl trifluoroacetyl, tert-butoxycarbonyl, trimethylsilyl, and 2-trimethylsilyl-ethanesulfonyl. Representative examples of non-aromatic end capping moieties suitable for C-terminus modification include, without limitation, amides, allyloxycarbonyl, trialkylsilyl ethers and allyl ethers.

According to some embodiments, the end-capping modified peptides comprise dipeptides, and according to some embodiments, are homodipeptides (e.g., according to any of the respective embodiments described herein). In some such embodiments, the dipeptides are aromatic dipeptides, in which at least one, preferably both, of the amino acid residues is an aromatic amino acid residue.

Representative examples of such end-capping modified homodipeptides include, without limitation, end-capping modified phenylalanine-phenylalanine (Phe-Phe) dipeptides, end-capping modified naphthylalanine-naphthylalanine (Nal-Nal) dipeptides, end-capping modified (pentafluoro-phenylalanine)-(pentafluoro-phenylalanine) dipeptides, end-capping modified (iodo-phenylalanine)-(iodo-phenylalanine), end-capping modified (4-phenyl phenylalanine)-(4-phenyl phenylalanine) and end-capping modified (p-nitro-phenylalanine)-(p-nitro-phenylalanine). Fmoc-diphenylalanine (i.e., diphenylalanine which Fmoc attached to the N-terminus thereof) is an exemplary dipeptide which comprises phenylalanine and an end-capping moiety.

Contemplated are homodipeptides, and more preferably aromatic homodipeptides in which each of the amino acids comprises an aromatic moiety, such as, but not limited to, substituted or unsubstituted naphthalenyl and substituted or unsubstituted phenyl. The aromatic moiety can alternatively be substituted or unsubstituted heteroaryl such as, for example, indole, thiophene, imidazole, oxazole, thiazole, pyrazole, pyridine, pyrimidine, quinoline, isoquinoline, quinazoline, quinoxaline, and purine

When substituted, the phenyl, naphthalenyl or any other aromatic moiety includes one or more substituents such as, but not limited to, alkyl, trihaloalkyl, alkenyl, alkynyl, cycloalkyl, aryl, heteroaryl, heteroalicyclic, halo, nitro, azo, hydroxy, alkoxy, thiohydroxy, thioalkoxy, cyano, and amine.

In some of any of these embodiments, the end-capping modified peptide is an N-terminus modified peptide.

In some of any of these embodiments, the end-capping modified peptide comprises an aromatic end-capping moiety, as described herein.

In some of any of the embodiments described herein, the aromatic end-capping moiety is Fmoc.

In some of any of the embodiments described herein, at least one, or at least a portion, or each structure in the composition is formed, and is made, of a plurality of end-capping modified aromatic amino acids as described herein in any of the respective embodiments.

In some of any of the embodiments described herein, at least one, or at least a portion, or each structure in the composition is formed, and is made, of a plurality of end-capping modified aromatic dipeptides (in which at least one, preferably both, of the amino acid residues is an aromatic amino acid residue).

In some of any of the embodiments described herein, at least one, or at least a portion, or each structure in the composition is formed, and is made, of a plurality of aromatic amino acids.

In some of any of the embodiments described herein, the aromatic amino acid is phenylalanine.

In some of any of the embodiments described herein, at least one, or at least a portion, or each structure in the composition is formed, and is made, a plurality of phenylalanine molecules.

The phrase “aromatic dipeptide” describes a peptide composed of two amino acid residues, at least one, and preferably both, being an aromatic amino acid as defined herein.

The phrase “end-capping modified dipeptide”, as used herein, refers to a dipeptide as described herein which has been modified at the N(amine)-terminus and/or at the C(carboxyl)-terminus thereof. The end-capping modification refers to the attachment of a chemical moiety to the terminus, so as to form a cap. Such a chemical moiety is referred to herein as an end-capping moiety and is typically also referred to herein and in the art, interchangeably, as a peptide protecting moiety or group.

In a preferred embodiment of the present invention, the end-capping modified dipeptides are modified by an aromatic (e.g. Fmoc) end-capping moiety.

The end-capping moieties described herein for N-terminus modification can also be utilized for providing an amine-modified aromatic amino acid as described herein.

In exemplary embodiments, at least a portion, and preferably each, of the plurality of peptides is fluorenylmethyloxycarbonyl-diphenylalanine (FmocFF), wherein each of the phenylalanine residues is independently an L-phenylalanine or D-phenylalanine. In exemplary embodiments, the peptide is Fmoc(L)F(L)F).

The plurality of peptides that form the self-assembled structure as described herein are also referred to herein as “hydrogelators”.

According to some of any of the embodiments described herein, the self-assembled structure (e.g., hydrogel) is obtainable upon contacting the plurality of peptides with an aqueous solution (e.g., water or buffer) under conditions that allow or promote the self-assembly, as previously described. Exemplary conditions are described in the Examples section that follows.

According to some of any of the embodiments described herein, the self-assembled structure (e.g., hydrogel) is obtained upon contacting the plurality of peptides with an aqueous solution (e.g., water or buffer) of the plurality of peptides under conditions that allow or promote the self-assembly, as previously described, with an electrode as described herein, such that the self-assembled structure is formed in the presence of the electrode and is thereby associated therewith.

In some of any of the embodiments described herein, a concentration of the peptides (according to any of the respective embodiments described herein) in the solution (according to any of the respective embodiments described herein) is no more than 20 mg/ml (e.g., ranges from 1 to 20 mg/ml or from 5 to 20 mg/ml, including any intermediate values and subranges therebetween), or no more than 15 mg/ml (e.g., ranges from 1 to 15 mg/ml or from 5 to 15 mg/ml or from 10 to 15 mg/ml, including any intermediate values and subranges therebetween), or no more than 10 mg/ml (e.g., ranges from 1 to 10 mg/ml or from 2 to 10 mg/ml or from 5 to 10 mg/ml, including any intermediate values and subranges therebetween), or no more than 5 mg/ml (e.g., from 1 to 5 mg/ml or from 2 to 5 mg/ml), and is optionally about 5 mg/ml.

The contacting is performed under conditions that effect self-assembly of the plurality of peptides, as described hereinafter in further detail.

According to some of any of the embodiments described herein, a concentration of the plurality of peptides that form the self-assembled structure in the composition-of-matter is in a range of from 0.1 mg/mL to 100 mg/mL, or from 0.1 mg/mL to 50 mg/mL, or from 0.1 mg to 20 mg/mL, or from 0.1 mg to 10 mg/mL, or from 1 mg/mL to 100 mg/mL, or from 1 mg/mL to 50 mg/mL, or from 1 mg/mL to 20 mg/mL, preferably from 1 mg/mL to 10 mg/mL or from 1 mg/mL to mg/mL, including any intermediate values and subranges therebetween.

In exemplary embodiments, the concentration of the plurality of peptides that form the self-assembled structure in the composition-of-matter ranges from 1 gram to 100 gram, or from 1 gram to 50 gram, or from 1 gram to 20 grams, including any intermediate values and subranges therebetween, per a total surface area of the electrode or of an array of two or more electrodes of 1 m².

According to some of any of the embodiments described herein, a volume of the self-assembled structure (including a biocatalyst and optionally an electron transfer mediator or any other additive) in the composition-of-matter ranges from 0.1 mL to 100 mL, or from 0.1 to 80 mL, or from 0.1 to 50 mL, or from 0.1 to 40 mL, or from 0.1 to 20 mL, or from 0.1 to 10 mL, or from 0.1 to 5 mL, or from 0.1 to 2 mL, or from 1 to 20 mL, or from 1 to 10 mL, or from 1 to 4 mL, including any intermediate values and subranges therebetween, per a total surface area of the electrode or of an array of two or more electrodes of 2 cm².

According to some of any of the embodiments described herein, a volume of the self-assembled structure (including a biocatalyst and optionally an electron transfer mediator or any other additive) in the composition-of-matter ranges from 0.1 L to 100 L, or from 0.1 to 80 L, or from 0.1 to 50 L, or from 0.1 to 40 L, or from 0.1 to 20 L, or from 0.1 to 10 L, or from 0.1 to 5 L, or from 1 to 20 L, or from 1 to 10 L, including any intermediate values and subranges therebetween, per a total surface area of the electrode or of an array of two or more electrodes of 1 m².

### Biocatalyst:

The term “biocatalyst” describes a substance of a biological origin, typically a protein, which accelerates, promotes or catalyzes a biochemical reaction, mostly in living organisms. Biocatalysts are derived from biological systems and are typically enzymes. As such, biocatalysts are highly specific, and are employed in biotechnology and industrial processes to enhance the efficiency, selectivity, and environmental sustainability of chemical transformations.

According to some of any of the embodiments described herein, the biocatalysts is an enzyme.

According to some of any of the embodiments described herein, the biocatalysts is an enzyme-producing or enzyme-expressing microorganism.

According to some of any of the embodiments described herein, the biocatalyst (e.g., enzyme) catalyzes or participates in a redox reaction, either alone or in the presence of a co-reactant.

A redox reaction, or a reduction-oxidation reaction, is a type of chemical reaction in which electrons are transferred between two chemical species. These reactions involve a change in the oxidation states of the reacting elements, where one element undergoes oxidation (loses electrons) and another undergoes reduction (gains electrons). The term “redox” is derived from the two simultaneous processes of reduction and oxidation occurring in the same reaction.

Some enzymes that participate in redox reactions may comprise a transition metal such as iron, for example, in the form of one or more iron-sulfur cluster and/or di-iron (e.g., di-iron azadithiolate) center.

According to some of any of the embodiments described herein, the biocatalyst (e.g., enzyme) catalyzes a redox reaction that produces or consumes a gas such as hydrogen gas (H₂), oxygen gas (O₂), nitrogen gas (N₂), carbon dioxide (CO₂).

Exemplary enzymes that are usable in the context of the present embodiments include, but are not limited to, a hydrogenase, a nitroreductase, a nitrogenase, ferredoxin-NADP+ reductase (FNR) and a Cytochrome P450 enzyme. Additional examples include, but are not limited to, laccase, alcohol dehydrogenase, sulfite reductase, and pyruvate ferredoxin oxidoreductase. Any other enzymes that are usable in catalyzing industrial redox reactions are contemplated.

In some of any of the embodiments described herein, the biocatalyst comprises an [FeFe]-hydrogenase, for example, a Chlamydomonas reinhardtii [FeFe]-hydrogenase.

Microorganisms, or portions thereof, that produce or express one or more the enzymes as described herein can be used as biocatalysts.

The term “microorganism” as used herein and in the art describes an organism that is typically microscopic (too small to be seen by the naked human eye) and/or unicellular.

Microorganisms are very diverse and include bacteria, fungi, archaea, and protists; microscopic plants (called green algae); and animals such as plankton, the planarian and the amoeba.

In exemplary embodiments, the microorganism is selected from bacteria and fungi, and in some embodiments, the microorganism is a bacterium.

Examples of whole microorganisms that could be used to express enzymes in accordance with the present invention include, but are not limited to, bacterium such as E. coli, Clostridium spp, Dehalococcoides, Desulfovibrio spp, Streptomyces spp, Rhodobacter capsulatus sphaeroides, Saccharomyces cerevisiae. Any other enzyme-expressing bacteria or fungi, for example bacteria or fungi that express enzymes that are usable in catalyzing redox reactions are contemplated.

According to some of any of the embodiments described herein, the biocatalyst is an enzyme as described herein, and its concentration in the composition-of-matter is in a range of from 1 microgram/mL to 1,000 micrograms/mL, or from 1 microgram/mL to 800 micrograms/mL, or from 1 microgram/mL to 600 micrograms/mL, or from 1 microgram/mL to 500 micrograms/mL, or from 1 microgram/mL to 400 micrograms/mL, or from 1 microgram/mL to 300 micrograms/mL, or from 1 microgram/mL to 200 micrograms/mL, or from 10 micrograms/mL to 1,000 micrograms/mL, or from 10 micrograms/mL to 800 micrograms/mL, or from 10 micrograms/mL to 600 micrograms/mL, or from 10 micrograms/mL to 500 micrograms/mL, or from 10 micrograms/mL to 400 micrograms/mL, or from 10 micrograms/mL to 300 micrograms/mL, or from 10 micrograms/mL to 200 micrograms/mL, or from 100 micrograms/mL or 1,000 micrograms/mL, or from 100 micrograms/mL to 800 micrograms/mL, or from 100 micrograms to 600 micrograms, or from 100 micrograms to 500 micrograms, or from 100 micrograms/mL to 400 micrograms/mL, or from 100 micrograms/mL to 300 micrograms/mL, or from 100 micrograms/mL to 200 micrograms/mL, or from or from 1 microgram/mL or 100 micrograms/mL, or from 10 micrograms/mL to 100 micrograms/mL, or from 1 microgram/mL to 50 micrograms/mL, including any intermediate values and subranges therebetween.

According to some of any of the embodiments described herein, the biocatalyst is an enzyme as described herein, and its concentration in the composition-of-matter is in a range of from 1 to 1,000, or from 1 to 100, or from 1 to 20, or from 5 to 50, or from 5 to 20, or from 5 to 15, or from 1 to 10, or from 0.1 to 100, or from 0.1 to 10, ×10¹ cells/mL, including any intermediate values and subranges therebetween.

According to some of any of the embodiments described herein, the biocatalyst is an enzyme as described herein, and its concentration in the composition-of-matter is such that exhibits OD of at least 2, or of at least 3, for example, an OD value in a range of from 2 to 20, or from 2 to 10, or from 2 to 5, or from 3 to 10, or from 3 to 8, or from 3 to 5, or from 2 to 4, including any intermediate values and subranges therebetween. OD can be determined by any method known in the art, an example of which is provided in the Examples section that follows.

### Electron Transfer Mediator:

According to some of any of the embodiments described herein, the composition-of-matter further comprises an electron transfer mediator.

According to some of any of the embodiments described herein, the electron transfer mediator is associated with, as described herein (e.g., entrapped, encapsulated or embedded therein).

Herein, the phrase “electron transfer mediator” describes a substance which facilitates electron transfer by reversibly accepting and donating electrons. The electron transfer mediator has both a relatively stable reduced form and a relatively stable oxidized form in order to be capable of cycling between both such states.

In some embodiments, at least one relatively stable reduced form and at least one relatively stable oxidized form differ by a single electron, such that the mediator can facilitate transfer of single electrons. Examples of structures suitable for mediating transfer of single electrons (e.g., having a stabilized form generated by acceptance or donation of a single electron, such as a stable radical anion or radical cation) include, without limitation, transition metals and conjugated pi-electron systems (e.g., such as polycyclic aromatic systems and conjugated aromatic rings), especially pi-electron systems comprising heteroatoms (e.g., heteroaryl).

The electron transfer mediator is optionally soluble in a hydrogel as described herein, e.g., in an aqueous medium forming the hydrogel. Solubility of the mediator may enhance electron transfer, for example, by enhancing diffusion of the mediator between an electron donor and electron acceptor.

In some of any of the embodiments described herein, the electron transfer mediator comprises a viologen. Methyl viologen (also known in the art as “paraquat”) is an exemplary viologen, e.g., in a form of a chloride salt. In some of any of these embodiments, the biocatalyst is an enzyme as described herein in any of the respective embodiments and any combination thereof.

Herein, the term “viologen” refers to any compound having a substituted or unsubstituted bipyridine (preferable 4,4′-bipyridine) structure (including salts thereof), for example, wherein one or both nitrogen atoms are substituted by alkyl (e.g., methyl), alkenyl, alkynyl, cycloalkyl, aryl, heteroaryl and/or heteroalicyclic so as to form a positively charged tetravalent nitrogen atom.

In some of any of the embodiments described herein, a concentration of electron transfer mediator (e.g., viologen) in the self-assembled structure according to any of the respective embodiments is at least 0.1 mM, optionally in a range of from 0.1 to 20 mM, optionally in a range of from 0.1 to 5 mM, and optionally in a range of from 1 to 5 mM, including any intermediate values and subranges therebetween.

In some of any of the embodiments described herein, a total amount of electron transfer mediator (e.g., viologen) in the self-assembled structure according to any of the respective embodiments is in a range of from 0.1 to 20 mg, or from 0.1 to 10 mg, or from 0.1 to 5 mg, or from 1 to 20, or from 1 to 10, or from 1 to 5, or from 2 to 3 mg, including any intermediate values and subranges therebetween.

Additional examples of electron transfer mediators include, but are not limited to, carbon nanotubes, polyaniline, and poly(3,4-ethylenedioxythiophene) polystyrene sulfonate (PEDOT:PSS).

Additional examples of electron transfer mediators include, but are not limited to, two-dimensional (2D) nanomaterials, which are a subtype of nanomaterials with ultrathin layer-structured topology, such as graphene and its derivatives, transition metal dichalcogenides (TMDCs), transition metal oxides transition metal oxides (TMOs), and transition metal carbides (MXenes).

According to some of any of the embodiments described herein the electron transfer mediator is an MXene. According to some of these embodiments, the biocatalyst is a microorganism, as described herein in any of the respective embodiments.

MXenes include 2D inorganic materials with ultrathin atomic thicknesses that are composed of layered transition metal carbides and either nitrides or carbonitrides. MXenes can be collectively represented by the formula Mn+1XnTx, where M is an early transition metal (e.g., Ti, Nb, Cr, Ta, V, Sc, or Mo); X is carbon and/or nitrogen; n is 1 to 3; and Tx represents the chemical groups that represents the surface functionalization (e.g., O, OH, F, and/or Cl). Reference is made, for example, to Li et al., Journal of Nanobiotechnology, volume 21, Article number: 73 (2023), which describes a plethora of MXene that are usable in the context of the embodiments of the present invention, and which is incorporated by reference as if fully set forth herein.

According to some of any of the embodiments described herein, the MXene is a carbide, such that X is C, although nitrides, in which X is N are also contemplated.

According to some of any of the embodiments described herein, the metal M is the MXene is titanium or molybdenum, preferably titanium (Ti).

In exemplary embodiments, the MXene is Ti3C2Tx.

In exemplary embodiments, Tx represents a mixture of functional surface groups, for example, a mixture of hydroxy, oxygen and/or halo surface groups.

According to some of any of the embodiments described herein, when an electron transfer mediator is an inorganic material (e.g., MXene), it is added to the composition-of-matter as particles, preferably nanoparticles and/or microparticles.

According to some of these embodiments, the average particles size (e.g., diameter) is in a range of from 0.1 to 100 microns, or 0.1 to 50 microns, or 0.1 to 20 microns, or 0.2 to 100 microns, or 0.1 to 50 microns, or 0.1 to 20 microns, or 0.1 to 10 microns, or 0.1 to 5 microns, or 0.1 to 2 microns, or 0.1 to 50 microns, or 0.5 to 20 microns, or 0.5 to 10 microns, or 0.5 to 5 microns, including any intermediate values and subranges therebetween, or is about 1 micron.

### Process:

According to an aspect of some embodiments of the present invention there is provided a process of preparing composition-of-matter as described herein in any of the respective embodiments. The process can also be referred to as a process of preparing a modified electrode as described herein, or as a process of modifying an electrode.

According to some embodiments, the process is effected by contacting an electrode as described herein in any of the respective embodiments and any combination thereof, with a solution comprising the plurality of peptides and with a solution comprising the biocatalyst.

According to some embodiments, the composition-of-matter further comprises an electron transfer mediator, and the contacting is further with a solution that comprises the electron transfer mediator. In some embodiments, the electron transfer mediator is included in one of the solutions, of the peptides or of the biocatalyst, or in both. In some embodiments, the electron transfer mediator is included in the biocatalyst solution.

According to some embodiments, the solutions are mixed with one another prior to contacting the electrode, such that the contacting is subsequent to mixing the solution is performed by contacting the electrode with a mixed solution comprising the plurality of peptides and the biocatalysts (and optionally an electron transfer mediator).

According to some of any of the embodiments described herein, a self-assembled structure as described herein is formed upon contacting a stock solution of a plurality of peptides in a water-miscible organic solvent with an aqueous solution. According to some of any of the embodiments described herein, the self-assembled structure if formed in a solution comprising same (e.g., upon contacting a stock solution of a plurality of peptides in a water-miscible organic solvent with an aqueous solution) and this solution is mixed with a solution comprising the biocatalyst and optionally an electron transfer mediator. According to some of these embodiments, a concentration of the peptides in the aqueous solution where the self-assembled structure is formed is such that the self-assembled structure features a liquid consistency (e.g., a storage modulus lower than 1 Pa). In some of these embodiments, the concentration is up or is about to 2 mg/mL.

Preferably, the self-assembled structure is formed by mixing a stock solution of a plurality of peptides in a water-miscible organic solvent with an aqueous solution that comprises the biocatalyst and optionally an electron transfer mediator.

The mixing is preferably at room temperature, or at a temperature that ranges between 1° and 30° C., or 20 to 30° C., or 20 to 25° C.

According to some embodiments, mixing the solutions prior to contacting the electrode, or when contacting the electrode, is such that a final concentration of the peptides in the aqueous solution is of at least 1 mg/ml, or at least 2 mg/ml, or at least 2.5 mg/ml, preferably at least 3 mg/ml, preferably at least 4 mg/ml, and up to 10 mg/ml, preferably up to 5 mg/mL, including any intermediate values and subranges therebetween.

According to some embodiments, mixing the solutions prior to contacting the electrode, or when contacting the electrode, is such that a final concentration of the biocatalyst in the aqueous solution is in a range of from 1 microgram/mL to 1,000 micrograms/mL, or from 1 microgram to 800 micrograms/mL, or from 1 microgram/mL to 600 micrograms/mL, or from 1 microgram/mL to 500 micrograms/mL, or from 1 microgram/mL to 400 micrograms/mL, or from 1 microgram/mL to 300 micrograms/mL, or from 1 microgram/mL to 200 micrograms/mL, or from 10 micrograms/mL to 1,000 micrograms/mL, or from 10 micrograms/mL to 800 micrograms/mL, or from 10 micrograms/mL to 600 micrograms/mL, or from 10 micrograms/mL to 500 micrograms/mL, or from 10 micrograms/mL to 400 micrograms/mL, or from 10/mL micrograms to 300 micrograms/mL, or from 10 micrograms/mL to 200 micrograms/mL, or from 100 micrograms/mL or 1,000 micrograms/mL, or from 100 micrograms/mL to 800 micrograms/mL, or from 100 micrograms/mL to 600 micrograms/mL, or from 100 micrograms/mL to 500 micrograms/mL, or from 100 micrograms/mL to 400 micrograms/mL, or from 100 micrograms/mL to 300 micrograms/mL, or from 100 micrograms/mL to 200 micrograms/mL, or from or from 1 microgram/mL or 100 micrograms/mL, or from 10 micrograms/mL to 100 micrograms/mL, or from 1 micrograms/mL to 50 micrograms/mL, including any intermediate values and subranges therebetween.

Accordingly, stock solutions of the plurality of peptides (e.g., a water-miscible organic solvent) and of the biocatalyst (and optionally the electron transfer mediator) can be pre-prepared and mixed at a ratio that provides the final concentrations as described herein.

Contacting a solution of the peptides dissolved in an organic solvent as described herein with an aqueous solution can be regarded as diluting the peptide's solution to a concentration that allows self-assembly of the peptides.

Mixing can be performed, for example, by manual or mechanical shaking (e.g., by vortex), or by magnetic or mechanical stirring.

The phrase “water-miscible organic solvent”, as used herein, refers to organic solvents that are soluble or miscible in water (e.g., when mixed with water at equal volumes at room temperature). Several factors inherent in the structure of the solvent molecules can affect the miscibility of organic solvents in water, such as for example, the length of the carbon chain and the type of functional groups therein. Hydrogen bonding plays a key role in making organic solvents miscible in water. For example, in alcohols, the hydroxyl group can form hydrogen bonding with water molecules. In addition, aldehydes, ketones and carboxylic acids can form hydrogen bonding via the carbonyl oxygen. Hydrogen bonding between ether and water molecules is also possible, enabling some degree of miscibility of simple ethers in water.

Examples of water-miscible organic solvents include, without limitation, simple alcohols, such as, methanol, ethanol, 1-propanol, 2-propanol, 1-butanol, 2-butanol, 2-methyl-1-propanol, 2-methyl-2-propanol, 2,2-dimethyl-1-propanol and their halogen substituted analogues, ethylene glycol, acetone, dimethylsulfoxide, acetic acid diethyl ether, tetrahydrofuran etc.

Representative examples of organic solvents that were successfully practiced in generating exemplary hydrogels according to the present invention include, acetone, dimethylsulfoxide and hexafluoroisopropanol (e.g., 1,1,1,3,3,3-hexafluoro-2-propanol, abbreviated herein as HFIP).

In some embodiments, the organic solvent is dimethylsulfoxide (abbreviated DMSO).

According to some of any of the embodiments described herein, contacting the solutions (e.g., subsequent to mixing the solutions) with the electrode is performed at room temperature.

According to some of any of the embodiments described herein, contacting the solutions (e.g., subsequent to mixing the solutions) with the electrode is performed for a time period of at least 1 hour, or at least 2 hours, e.g., in accordance with the time at which the self-assembled structure is formed.

According to some of any of the embodiments described herein, contacting the solutions (e.g., subsequent to mixing the solutions) with the electrode is effected by placing the electrode in a mixture of the solutions.

According to some of any of the embodiments described herein, when the solutions are mixed prior to contacting with the electrode, the contacting with the electrode is effect immediately (e.g., from 1 second and up to 10 minutes) after the solutions are mixed.

When an electron transfer mediator as described is included in one of the solutions, its concentration is the solution is such that provides a final concentration in the self-assembled structure (e.g., hydrogel) as described herein in any of the respective embodiments. In exemplary embodiments, the concentration in the biocatalyst solution is in range of from 0.1 to 10 mM, or from 1 to 5 mM, including any intermediate values and subranges therebetween.

According to some of the present embodiments, there is provided a modified electrode, prepared by the process as described herein in any of the respective embodiments and any combination thereof.

According to some of the present embodiments, a process as described herein can be regarded as a process of immobilizing the biocatalyst as described herein in any of the respective embodiments and any combination thereof to an electrode.

### Electrochemical System:

A modified electrode as described herein in any of the respective embodiments can be assembled into an electrochemical system, where it is connected to power source. The electrochemical system can be an electrochemical cell, or a system, for example, a system that is operated by the electrochemical cell or a product thereof.

According to an aspect of some embodiments of the present invention, there is provided an electrochemical cell comprising, as a working electrode, the composition-of-matter as described herein in any of the respective embodiments and any combination thereof, and a power source. According to some embodiments, the electrochemical cell is operated by connecting the electrode to the power source and applying potential to the electrode.

The electrochemical cell typically further comprises a reference electrode and optionally an auxiliary electrode.

The electrochemical cell further comprises electrically conducting elements that electrically connect the working electrode, the power source and the reference electrode, if present, and optionally the auxiliary electrode, if present.

The following describes some embodiments of an electrochemical cell of the invention.

In some embodiments, the modified, working electrode is electrically connectable to a power source, and the cell is configured such that when it is operated, at least a portion of the electrode contacts an electrolyte solution as described herein.

In some embodiments, the electrochemical cell further comprises a reference electrode. Any commercially available or customarily designed reference electrode is contemplated. In some embodiments, the reference electrode is an aqueous reference electrode. Exemplary usable reference electrodes include, but are not limited to, Silver/Silver Chloride electrode (e.g., Ag/AgCl/Saturated KCl electrode such as marketed by Metrohm), a Standard calomel (e.g., saturated calomel) electrode (SCE), a Standard hydrogen electrode (SHE), a Normal hydrogen electrode (NHE), a Reversible hydrogen electrode (RHE), a Copper-copper(II) sulfate electrode (CSE); a pH-electrode; a Palladium-hydrogen electrode, a Dynamic hydrogen electrode (DHE), and a Mercury-mercurous sulfate electrode (MSE).

The reference electrode is also electrically connectable, or electrically connected, to a power source, and the cell is configured such that when it is operated, a potential difference (voltage) is applied between the sensing electrode and the reference electrode. In some embodiments, the voltage is in a range of from 1 to 10, or from 1 to 5, Volts.

In some embodiments, the electrochemical cell follows a three-electrode design and further comprises an auxiliary electrode. Preferably, but not obligatory, the auxiliary electrode is a platinum electrode. Any other auxiliary electrode, commercially available or customarily designed, is contemplated. Non-limiting examples include gold electrodes, carbon electrodes and carbon/gold electrodes.

In some embodiments, the auxiliary electrode is electrically connectable, or electrically connected, to the working electrode.

In some of any of the embodiments described herein, the electrochemical cell further comprises a device that measures a current generated at the working electrode, as a result of a redox reaction that occurs at or next to (in the vicinity of) the working electrode. In some embodiments, this device (e.g., a currently collector, an amperometer) is electrically connectable to, or electrically connected to, the auxiliary electrode and the working electrode.

A schematic presentation of an exemplary assembly of a two-electrode electrochemical cell 10 according to some embodiments of the present invention is presented in FIG. 14.

Electrochemical cell 10 comprises a modified electrode 12 as described herein, which acts as a working electrode. Sensing electrode 12 has self-assembled structure 16 as described herein associated with at least on a portion thereof. When the cell is operated, the portion of the electrode associated with the self-assembled structure 16 should be in contact with an electrolyte 18, which preferably comprises a substrate of the biocatalyst. Working electrode 12 is one half of electrochemical cell 10. A reference electrode 22 is the other half of cell 10. A power source 20 is electrically connectable or connected to working electrode 12 and reference electrode 22 by means of electrical wires 24. Power source 20 is configured to apply voltage between sensing electrode 12 and reference electrode 22, for example, by applying potential to one of the electrodes.

For an electrochemical cell (e.g., cell 10) to operate, at least the working electrode (electrode 12) should be in contact with an electrolyte shown in FIG. 14 as an electrolyte 18. The electrochemical cell (e.g., cell 10) can comprise an electrolyte (e.g., electrolyte 18, as exemplified in FIG. 14), or can comprise means (e.g., an inlet port; not shown in FIG. 14), for introducing the electrolyte to the cell, so as to contact at least the working electrode (e.g., working electrode 12).

An electrochemical cell according to the present embodiments can follow any of the designs known in the art, and can include one or more sensing electrode(s), and one or more of a reference electrode(s) and/or an auxiliary electrode(s). Exemplary designs include, without limitation, rotating disk-ring electrodes, ultramicro-electrodes, or screen printed electrodes.

The configuration of the components of electrochemical cell 10 as presented in FIG. 14 are for illustrative purpose only and are not to be regarded as limiting in any way.

Electrochemical cell 10 can be, for example, in a form of a covered glass (or other inert material like Teflon or quartz) beaker, containing the sample solution in which the three electrodes are dipped. In some embodiments, electrochemical cell 10 is a micro cell or a thin layer cell.

Electrochemical cell 10 may further comprise means for mixing/stirring a sample with electrolyte 18 (not shown in FIG. 14).

Electrochemical cell 10 may further comprise means for monitoring and/or controlling the temperature inside the cell (not shown in FIG. 14).

As used herein and in the art, an electrolyte is an electrically conducting material or medium. An electrolyte can be solid or fluid, and can be used per se or when dissolved in a polar solvent, such as water. When dissolved is a solvent, it is referred to as an electrolyte solution.

Herein throughout, the term “electrolyte” also encompasses an “electrolyte solution”.

In an electrochemical cell as described herein (e.g., cell 10, FIG. 14), at least the working electrode (e.g., electrode 12) contacts the electrolyte (e.g., electrolyte 18) when the cell is operated. In some embodiments, all electrodes contact an electrolyte (e.g., electrolyte 18) when the cell is operated. In some embodiments, all electrodes contact the same electrolyte, as exemplified in FIG. 14, and in some embodiments, one or more of the electrodes contact an electrolyte different from the electrolyte in contact with the sensing electrode, and a membrane is interposed between the different electrolytes.

In some of any of the embodiments described herein, the electrolyte solution (e.g., electrolyte 18) is or comprises an aqueous solution.

In some of any of the embodiments described herein, the electrolyte solution (e.g., electrolyte solution 18, FIG. 14), features a pH suitable for a selected redox reaction to occur once the electrolyte contacts the working electrode (the biocatalyst immobilized to the electrode). In exemplary embodiments, the electrolyte solution features pH of between 6 and 8, or between 7 and 8, or about 7-7.5.

Exemplary aqueous solutions include buffer solutions, for example, phthalate buffer solutions, phosphate buffer solutions or any other buffer solution that provides the desired pH value, as described herein. Buffer solutions that provide pH values as described herein are well known to those skilled in the art. In exemplary embodiments, the aqueous solution further comprises additional salts or otherwise substances that enhance its electric conductivity, yet do not affect the activity of the biocatalyst (e.g., are biocompatible).

In some of any of the embodiments described herein, an electrolyte solution (e.g., electrolyte solution 18, FIG. 14), comprises a soluble salt (e.g., a water-soluble salt, or a salt soluble in the solvent mixture making up the electrolyte solution). Any soluble salt commonly used in electrolyte solution for increasing the ionic strength is contemplated, typically an inorganic salt. A concentration of the salt typically determines, at least in part, the ionic strength of the electrolyte solution.

In exemplary embodiments, an electrolyte solution (e.g., electrolyte solution 18, FIG. 14) which is an aqueous solution as described herein is a substrate of the biocatalyst.

According to some of any of the embodiments described herein, the electrochemical cell comprises a plurality of working electrodes, wherein at least one, at least two and preferably all the working electrodes are a modified electrode as described herein, and the working electrodes are all electrically connected to a power source, and optionally to one or more reference electrodes and further optionally to one or more auxiliary electrodes.

According to some any of the embodiments described herein, provided is a plurality of electrochemical cells, at least one, or more, of the electrochemical cells being an electrochemical cell comprising the modified electrode according to any of the respective embodiments and any combination, wherein the plurality of cells are electrically connected to one another, such that the cells operate simultaneously and the product of the reaction is assembled altogether from the plurality of cells.

By “plurality” it is meant herein throughout, two or more, for example, 2 to 100, or 2 to 50, or 2 to 20, or 2 to 10, or 10 to 100, or 10 to 50, or 10 to 20, or 5 to 50, or 5 to 20, or 5 to 15, or 5 to 10, including any intermediate values and subranges therebetween.

According to some of any of the embodiments described herein, the electrochemical cell further comprises a component configured for collecting a product of the electrochemical cell when operated.

In some embodiments, the product is electric energy, for example, a current, and the component is a current collector.

In some embodiments, the product is a gas and/or a liquid, and optionally a solid, and the electrochemical cell further comprises means for collecting the product once formed in the electrochemical cell.

In some embodiments, the electrochemical cell further comprises a reservoir configured for collecting a gas and/or liquid product formed in the electrochemical cell, for example, by reducing or oxidizing the substrate in the presence of the biocatalyst.

In some embodiments, the product is a gas usable as fuel in a fuel cell, for example, hydrogen or ammonia, and the electrochemical cell forms a part of a system that comprises the fuel cell, as described in further detail hereinafter.

According to some of any of the embodiments described herein, the electrochemical cell is operated in an inert environment, for example, in an oxygen-deficient environment, and further comprises means for effecting such an environment, for example, means for depleting oxygen or for introducing nitrogen or argon to the cell (not shown in FIG. 14).

According to an aspect of some embodiments of the present invention there is provided an electrochemical system comprising the electrochemical cell as described herein in any of the respective embodiments and any combination thereof.

The electrochemical system may comprise, in addition to the electrochemical cell, a system or device that is operable by the product of the electrochemical cell, and is in communication with the electrochemical cell, as described in further detail hereinunder.

The electrochemical system may comprise, in addition to the electrochemical cell, a reservoir configured for collecting a gas and/or liquid product formed in the electrochemical cell, for example, by reducing or oxidizing the substrate in the presence of the biocatalyst.

The electrochemical system can comprise a plurality of electrochemical cells as described herein in any of the respective embodiments and any combination thereof.

### Applications:

The modified electrode as described herein in any of the respective embodiments and any combination thereof can be integrated to or assembled with systems and devices which can benefit from the electrochemical reaction promoted by the biocatalyst.

The modified electrode as described herein allows performing redox reaction, and utilize the products of such reactions as described herein, in high efficiency (e.g., faradic efficiency), and under mild and cost-effective conditions, for example, using simple aqueous solutions such as saline or available buffer solutions, ambient temperatures, and/or physiological pH (of 6-8) throughout the manufacturing process of the electrode and the operation of electrochemical cell or systems or devices as described herein.

The modified electrode can be utilized continuously or intermittently, and is characterized by a life span that ranges from a few days to a few weeks, and even longer, by being able to maintain that catalytic activity of the biocatalyst.

For example, the electrode or electrochemical cell comprising same can be integrated in an energy conversion system or device, for energy conversion of electrical current to hydrogen, for reducing CO₂ chemical production, and any other system or device in which redox reactions can be utilized.

In some embodiments, the electrodes of the present embodiments or electrochemical cells comprising same can be used in devices or systems that convert electrical energy into H₂ gas. Such a device can be coupled with a fuel-cell and/or a gas storage

In some embodiments, the electrodes of the present embodiments or electrochemical cells comprising same can be used in devices or systems that convert electrical energy to ammonia.

The electrodes of the present embodiments or electrochemical cells comprising same can be used in devices or systems that convert gas to electrical energy, for example, for converting hydrogen gas or carbon dioxide to electric energy.

The electrodes of the present embodiments or electrochemical cells comprising same can be used in devices or systems that deplete CO₂ from an environment, to reduce its concentration in the environment, by using a biocatalyst that oxidize or reduce CO₂.

According to an aspect of some embodiments of the present invention there is provided a method of electrically producing a gas and/or liquid formed in a redox reaction catalyzed by a biocatalyst. The method according to these embodiments is effected by contacting a modified electrode as described herein in any of the respective embodiments and any combination thereof, which comprises a respective biocatalyst, with a substrate of the biocatalyst, such that the redox reaction between the biocatalyst and the substrate provided the gas and/or liquid when a potential is applied. The method is further effected by applying potential to the electrode, preferably, when the electrode is assembled in an electrochemical cell or system as described herein in any of the respective embodiments and any combination thereof.

According to an aspect of some embodiments of the present invention there is provided a method of performing a redox reaction of a substrate which is catalyzed by a biocatalyst. The method according to these embodiments is effected by contacting a modified electrode as described herein in any of the respective embodiments and any combination thereof, which comprises a respective biocatalyst, with the substrate of the biocatalyst, such that the redox reaction between the biocatalyst and the substrate occurs when a potential is applied. The method is further effected by applying potential to the electrode, preferably, when the electrode is assembled in an electrochemical cell or system as described herein in any of the respective embodiments and any combination thereof.

Applying potential can be effected by connecting the electrode to a power source.

In exemplary embodiments of the methods and systems as described herein, the biocatalyst comprises or generates a hydrogenase, and the methods and/or systems are for producing H₂ (e.g., “green” hydrogen). According to some of these embodiments, the substrate is or comprises water, preferably an aqueous solution such as a buffer or saline, as described herein in any of the respective embodiments.

In exemplary embodiments of the methods and systems as described herein, the biocatalyst comprises or generates a nitrogenase, and the methods and/or systems are for producing ammonia and/or a salt thereof.

When the product of the redox reaction is, for example, hydrogen, or ammonia, the obtained gas can be utilized as a fuel in a fuel cell system.

A fuel cell system typically comprises an anode compartment, a cathode compartment and a separator interposed therebetween. The fuel in a fuel cell is typically contacted with the anode compartment, where it undergoes a redox reaction that provides electrical energy.

According to an aspect of some embodiments of the present invention there is provided a fuel cell, operated by an electrochemical cell or system as described herein, that is, which is operated by a fuel generated by an electrochemical cell or system as described herein.

According to another aspect of embodiments of the invention there is provided a method of generating electricity, which is effected by electrochemically reacting a fuel, generated by an electrochemical cell or system as described herein, and an oxidant, as described herein.

In some embodiments, the method is effected by supplying a fuel generated by an electrochemical cell or system as described herein, as described herein, to the anode compartment of the fuel cell system, as described herein, and by supplying an oxidant composition, as described herein, to the cathode compartment of the system, as described herein, and by continuing supplying these components as long as electricity is required, and/or as desired, such that that system operates as a fuel cell.

Supplying the fuel to the respective compartments of the fuel cells can be effected by any means known in the art (e.g., via a pump, a pipe, etc.), that connects the electrochemical cell or system comprising the modified electrode of the present embodiments to the fuel cell system.

In some embodiments, the method is effected by utilizing the system as a battery, such that the fuel is supplied only before use.

Any of the fuel cell systems described herein can be used in many applications. Generally, the fuel cell can be incorporated in any electrically driven or hybrid electric (namely, driven by electrical and at least one additional form of energy) system or device, or can be in electrical communication with the system or device for operating it. Systems and devices incorporating the fuel cell as described herein can be stationary or movable, portable or non-portable. In some embodiments, the fuel cell system is incorporated in a power source which is adapted to power the electrically driven system or device. The size, shape and output of the fuel cell is preferably adapted to the application which consumes its energy.

Herein, the phrase “electrically driven system or device” and “electricity consuming system or device” are used interchangeably.

One type of application which can incorporate the fuel cell or portable power source according to some embodiments of the present invention is an electronic device. Representative examples of such device, include, without limitation, a portable telephone, a personal computer, a notebook computer, a portable charging dock, a pager, a PDA, a digital camera, a gameplayer, a smoke detector, a hearing aid, a portable TV, night vision goggles, a portable GPS device, a portable lighting device, a toy, a computer peripheral device, an household appliance, a cordless household appliance, an industrial product, a mobile equipment, a robot, a cordless tool (e.g., drill, saw).

Another type of application which can incorporate the fuel cell or portable power source according to some embodiments of the present invention is an electrically driven or hybrid electric vehicle. One example of a vehicle suitable for the present embodiments is an automobile such as, but not limited to, a car, a bus, a forklift, a segway, a motorcycle, a mobility scooter, a two-three- or four-wheel scooter, a saddle-ride type vehicle. Another example is an unmanned utility vehicle, such as, but not limited to, an autonomous lawn mower, an autonomous pool cleaner and the like. An additional example is an elevated altitude manned or unmanned vehicle, such as, but not limited to, an aircraft, a high altitude aircraft, a rocket and a spacecraft. A further example is a manned or unmanned underwater or above-water vehicle.

The fuel cell described herein can also be incorporated in distributed power source such as, but not limited to, a cogeneration system or a stationary power plant for a house, a public structure an industrial facility. Also contemplated are various appliances typical used in emergency situations, including, without limitation, emergency kits, emergency power supplies, emergency lights, backup generators and the like.

The fuel cell systems presented herein can be further used as a component in a power source in a location, such as, but not limited to, spacecraft, weather station, park, rural location and the like. A fuel cell system according to some embodiments of the present invention can be compact and lightweight.

The fuel cell systems presented herein can be further used in combined heat and power systems. The fuel cell systems of the present embodiments can be used to generate electric power, and at the same time produce hot air and water from the waste heat.

In some embodiments, the fuel cell systems described herein are utilized in a method for powering an electrically-driven or electricity-consuming system or device, as described herein. The powering is effected by establishing electrical communication (e.g., connecting) between the electrochemical cell or system as described herein and a fuel cell system, and between the fuel cell system and the electricity-consuming system or device.

As used herein the term “about” refers to ±10 or ±5%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

Herein, the term “hydrocarbon” describes an organic moiety that includes, as its basic skeleton, a chain of carbon atoms, substituted mainly by hydrogen atoms. The hydrocarbon can be saturated or non-saturated, be comprised of aliphatic, alicyclic or aromatic moieties, and can optionally be substituted by one or more substituents (other than hydrogen). A substituted hydrocarbon may have one or more substituents, whereby each substituent group can independently be, for example, cycloalkyl, alkenyl, alkynyl, aryl, heteroaryl, heteroalicyclic, amine, halide, sulfate, sulfonate, sulfonyl, sulfoxide, phosphate, phosphonyl, phosphinyl, hydroxy, alkoxy, aryloxy, thiohydroxy, thioalkoxy, thioaryloxy, oxo, cyano, nitro, azo, azide, sulfonamide, carbonyl, thiocarbonyl, carboxy, thiocarbamate, urea, thiourea, carbamate, amide, epoxide and hydrazine. The hydrocarbon can be an end group or a linking group, as these terms are defined herein.

Preferably, the hydrocarbon moiety has 1 to 20 carbon atoms. Whenever a numerical range; e.g., “1 to 20”, is stated herein, it implies that the group, in this case the alkyl group, may contain 1 carbon atom, 2 carbon atoms, 3 carbon atoms, etc., up to and including 20 carbon atoms.

Herein, the term “alkyl” describes a saturated aliphatic hydrocarbon end group, as defined herein, including straight chain and branched chain groups. Preferably, the alkyl group has 1 to 20 carbon atoms. More preferably, the alkyl is a medium size alkyl having 1 to 10 carbon atoms. Most preferably, unless otherwise indicated, the alkyl is a lower alkyl having 1 to 4 carbon atoms.

The alkyl group may be substituted or unsubstituted. Substituted alkyl may have one or more substituents, whereby each substituent group can independently be, for example, cycloalkyl, alkenyl, alkynyl, aryl, heteroaryl, heteroalicyclic, amine, halide, sulfate, sulfonate, sulfonyl, sulfoxide, phosphate, phosphonyl, phosphinyl, hydroxy, alkoxy, aryloxy, thiohydroxy, thioalkoxy, thioaryloxy, cyano, nitro, azo, azide, sulfonamide, carbonyl, thiocarbonyl, carboxy, thiocarbamate, urea, thiourea, carbamate, amide, epoxide and hydrazine.

The term “alkylene” describes a saturated or unsaturated aliphatic hydrocarbon linking group, as this term is defined herein, which differs from an alkyl group (when saturated) or an alkenyl or alkynyl group (when unsaturated), as defined herein, only in that alkylene is a linking group rather than an end group.

Herein, the term “alkenyl” describes an unsaturated aliphatic hydrocarbon end group which comprises at least one carbon-carbon double bond, including straight chain and branched chain groups. Preferably, the alkenyl group has 2 to 20 carbon atoms. More preferably, the alkenyl is a medium size alkenyl having 2 to 10 carbon atoms. Most preferably, unless otherwise indicated, the alkenyl is a lower alkenyl having 2 to 4 carbon atoms. The alkenyl group may be substituted or unsubstituted. Substituted alkenyl may have one or more substituents, whereby each substituent group can independently be, for example, cycloalkyl, alkynyl, aryl, heteroaryl, heteroalicyclic, amine, halide, sulfate, sulfonate, sulfonyl, sulfoxide, phosphate, phosphonyl, phosphinyl, hydroxy, alkoxy, aryloxy, thiohydroxy, thioalkoxy, thioaryloxy, cyano, nitro, azo, azide, sulfonamide, carbonyl, thiocarbonyl, carboxy, thiocarbamate, urea, thiourea, carbamate, amide, epoxide and hydrazine.

Herein, the term “alkynyl” describes an unsaturated aliphatic hydrocarbon end group which comprises at least one carbon-carbon triple bond, including straight chain and branched chain groups. Preferably, the alkynyl group has 2 to 20 carbon atoms. More preferably, the alkynyl is a medium size alkynyl having 2 to 10 carbon atoms. Most preferably, unless otherwise indicated, the alkynyl is a lower alkynyl having 2 to 4 carbon atoms. The alkynyl group may be substituted or unsubstituted. Substituted alkynyl may have one or more substituents, whereby each substituent group can independently be, for example, cycloalkyl, alkenyl, aryl, heteroaryl, heteroalicyclic, amine, halide, sulfate, sulfonate, sulfonyl, sulfoxide, phosphate, phosphonyl, phosphinyl, hydroxy, alkoxy, aryloxy, thiohydroxy, thioalkoxy, thioaryloxy, cyano, nitro, azo, azide, sulfonamide, carbonyl, thiocarbonyl, carboxy, thiocarbamate, urea, thiourea, carbamate, amide, epoxide and hydrazine.

The term “cycloalkyl” describes an all-carbon monocyclic or fused ring (i.e., rings which share an adjacent pair of carbon atoms) group where one or more of the rings does not have a completely conjugated pi-electron system. The cycloalkyl group may be substituted or unsubstituted. Substituted cycloalkyl may have one or more substituents, whereby each substituent group can independently be, for example, alkyl, alkenyl, alkynyl, cycloalkyl, aryl, heteroaryl, heteroalicyclic, amine, halide, sulfate, sulfonate, sulfonyl, sulfoxide, phosphate, phosphonyl, phosphinyl, hydroxy, alkoxy, aryloxy, thiohydroxy, thioalkoxy, thioaryloxy, cyano, nitro, azo, azide, sulfonamide, carbonyl, thiocarbonyl, carboxy, thiocarbamate, urea, thiourea, carbamate, amide, epoxide and hydrazine. The cycloalkyl group can be an end group, as this phrase is defined herein, wherein it is attached to a single adjacent atom, or a linking group, as this phrase is defined herein, connecting two or more moieties.

The term “aryl” describes an all-carbon monocyclic or fused-ring polycyclic (i.e., rings which share adjacent pairs of carbon atoms) end group (as this term is defined herein) having a completely conjugated pi-electron system. The aryl group may be substituted or unsubstituted.

Substituted aryl may have one or more substituents, whereby each substituent group can independently be, for example, alkyl, alkenyl, alkynyl, cycloalkyl, aryl, heteroaryl, heteroalicyclic, amine, halide, sulfate, sulfonate, sulfonyl, sulfoxide, phosphate, phosphonyl, phosphinyl, hydroxy, alkoxy, aryloxy, thiohydroxy, thioalkoxy, thioaryloxy, cyano, nitro, azo, azide, sulfonamide, carbonyl, thiocarbonyl, carboxy, thiocarbamate, urea, thiourea, carbamate, amide, epoxide and hydrazine. Phenyl and naphthyl are representative aryl end groups.

The term “heteroaryl” describes a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furan, thiophene, imidazole, oxazole, thiazole, pyrazole, pyridine, pyrimidine, quinoline, isoquinoline and purine. The heteroaryl group may be substituted or unsubstituted. Substituted heteroaryl may have one or more substituents, whereby each substituent group can independently be, for example, alkyl, alkenyl, alkynyl, cycloalkyl, aryl, heteroaryl, heteroalicyclic, amine, halide, sulfate, sulfonate, sulfonyl, sulfoxide, phosphate, phosphonyl, phosphinyl, hydroxy, alkoxy, aryloxy, thiohydroxy, thioalkoxy, thioaryloxy, cyano, nitro, azo, azide, sulfonamide, carbonyl, thiocarbonyl, carboxy, thiocarbamate, urea, thiourea, carbamate, amide, epoxide and hydrazine. The heteroaryl group can be an end group, as this phrase is defined herein, where it is attached to a single adjacent atom, or a linking group, as this phrase is defined herein, connecting two or more moieties.

Representative examples are pyridine, pyrrole, oxazole, indole, purine and the like.

The term “arylene” describes a monocyclic or fused-ring polycyclic linking group, as this term is defined herein, and encompasses linking groups which differ from an aryl or heteroaryl group, as these groups are defined herein, only in that arylene is a linking group rather than an end group.

The term “heteroalicyclic” describes a monocyclic or fused ring group having in the ring(s) one or more atoms such as nitrogen, oxygen and sulfur. The rings may also have one or more double bonds. However, the rings do not have a completely conjugated pi-electron system. The heteroalicyclic may be substituted or unsubstituted. Substituted heteroalicyclic may have one or more substituents, whereby each substituent group can independently be, for example, alkyl, alkenyl, alkynyl, cycloalkyl, aryl, heteroaryl, heteroalicyclic, amine, halide, sulfate, sulfonate, sulfonyl, sulfoxide, phosphate, phosphonyl, phosphinyl, hydroxy, alkoxy, aryloxy, thiohydroxy, thioalkoxy, thioaryloxy, cyano, nitro, azo, azide, sulfonamide, carbonyl, thiocarbonyl, carboxy, thiocarbamate, urea, thiourea, carbamate, amide, epoxide and hydrazine. The heteroalicyclic group can be an end group, as this phrase is defined herein, where it is attached to a single adjacent atom, or a linking group, as this phrase is defined herein, connecting two or more moieties.

Representative examples are piperidine, piperazine, tetrahydrofuran, tetrahydropyran, morpholine and the like.

As used herein, the terms “amine” and “amino” describe both a —NRxRy end group and a —NRx— linking group, wherein Rx and Ry are each independently hydrogen, alkyl, cycloalkyl, aryl, heteroaryl or heteroalicyclic, as these terms are defined herein. When Rx or Ry is heteroaryl or heteroalicyclic, the amine nitrogen atom is bound to a carbon atom of the heteroaryl or heteroalicyclic ring. A carbon atom attached to the nitrogen atom of an amine is not substituted by ═O or ═S, and in some embodiments, is not substituted by any heteroatom.

The amine group can therefore be a primary amine, where both Rx and Ry are hydrogen, a secondary amine, where Rx is hydrogen and Ry is alkyl, cycloalkyl, aryl, heteroaryl or heteroalicyclic, or a tertiary amine, where each of Rx and Ry is independently alkyl, cycloalkyl, aryl, heteroaryl or heteroalicyclic.

The terms “hydroxy” and “hydroxyl” describe a —OH group.

The term “alkoxy” describes both an —O-alkyl and an —O-cycloalkyl end group, or —O-alkylene or —O-cycloalkyl linking group, as defined herein.

The term “aryloxy” describes both an —O-aryl and an —O-heteroaryl end group, or an —O-arylene-linking group, as defined herein.

The term “thiohydroxy” describes a —SH group.

The term “thioalkoxy” describes both an —S-alkyl and an —S-cycloalkyl end group, or —S-alkylene or —S-cycloalkyl linking group, as defined herein.

The term “thioaryloxy” describes both an —S-aryl and an —S-heteroaryl end group, or an —S-arylene-linking group, as defined herein.

The terms “cyano” and “nitrile” describe a —C≡N group.

The term “nitro” describes an —NO₂ group.

The term “oxo” describes an ═O group.

The term “azide” describes an —N═N⁺═N⁻ group.

The term “azo” describes an —N═N—Rx end group or —N═N— linking group, with Rx as defined herein.

The terms “halide” and “halo” refer to fluorine, chlorine, bromine or iodine. In some of any of the respective embodiments, halo is fluoro (e.g., pentahalo is pentafluoro).

The term “phosphate” refers to a —O—P(═O)(ORx)—ORy end group, or to a —O—P(═O)(ORx)—O— linking group, where Rx and Ry are as defined herein, except when referring to a phosphate ion salt such as a calcium phosphate.

The terms “phosphonyl” and “phosphonate” refer to an —P(═O)(ORx)—ORy end group, or to a —P(═O)(ORx)—O— linking group, where Rx and Ry are as defined herein.

The term “phosphinyl” refers to a —PRxRy group, where Rx and Ry are as defined hereinabove.

The term “sulfoxide” or “sulfinyl” describes a —S(═O)—Rx end group or —S(═O)— linking group, where Rx is as defined herein.

The term “sulfonyl” describe a —S(═O)₂—Rx end group or —S(═O)₂— linking group, where Rx is as defined herein.

The term “sulfonate” describes a —S(═O)₂—O-Rx or —O—S(═O)₂—Rx end group or —S(═O)₂—O— linking group, where Rx is as defined herein.

The term “sulfate” describes a —O—S(═O)₂—O-Rx end group or —O—S(═O)₂—O— linking group, where Rx is as defined herein.

The terms “sulfonamide” and “sulfonamido”, as used herein, encompass both S-sulfonamide and N-sulfonamide end groups, and a —S(═O)₂—NRx— linking group.

The term “S-sulfonamide” describes a —S(═O)₂—NRxRy end group, with Rx and Ry as defined herein.

The term “N-sulfonamide” describes an RxS(═O)₂—NRy— end group, where Rx and Ry are as defined herein.

The term “carbonyl” as used herein, describes a —C(═O)—Rx end group or —C(═O)— linking group, with Rx as defined herein.

The term “aldehyde” herein describes a —C(═O)H end group.

The term “thiocarbonyl” as used herein, describes a —C(═S)—Rx end group or —C(═S)— linking group, with Rx as defined herein.

The terms “carboxy” and “carboxyl”, as used herein, encompasses both C-carboxy and O-carboxy end groups, and a —C(═O)—O— linking group.

The term “C-carboxy” describes a —C(═O)—ORx end group, where Rx is as defined herein.

The term “carboxylic acid” describes a —C(═O)—OH end group, or a deprotonated form (—CO₂) or salt thereof.

The term “O-carboxy” describes a —OC(═O)—Rx end group, where Rx is as defined herein.

The term “urea” describes a —NRxC(═O)—NRyRw end group or —NRxC(═O)—NRy— linking group, where Rx and Ry are as defined herein and Rw is as defined herein for Rx and Ry.

The term “thiourea” describes a —NRx-C(═S)—NRyRw end group or a —NRx-C(═S)—NRy-linking group, with Rx, Ry and Rw as defined herein.

The terms “amide” and “amido”, as used herein, encompasses both C-amide and N-amide end groups, and a —C(═O)—NRx— linking group.

The term “C-amide” describes a —C(═O)—NRxRy end group, where Rx and Ry are as defined herein.

The term “N-amide” describes a RxC(═O)—NRy— end group, where Rx and Ry are as defined herein.

The term “carbamyl” or “carbamate”, as used herein, encompasses N-carbamate and O-carbamate end groups, and a —OC(═O)—NRx— linking group.

The term “N-carbamate” describes a RyOC(═O)—NRx— end group, with Rx and Ry as defined herein.

The term “O-carbamate” describes an —OC(═O)—NRxRy end group, with Rx and Ry as defined herein.

The term “thiocarbamyl” or “thiocarbamate”, as used herein, encompasses O-thiocarbamate, S-thiocarbamate and N-thiocarbamate end groups, and a —OC(═S)—NRx- or —SC(═O)—NRx— linking group.

The terms “O-thiocarbamate” and “O-thiocarbamyl” describe a —OC(═S)—NRxRy end group, with Rx and Ry as defined herein.

The terms “S-thiocarbamate” and “S-thiocarbamyl” describe a —SC(═O)—NRxRy end group, with Rx and Ry as defined herein.

The terms “N-thiocarbamate” and “N-thiocarbamyl” describe a RyOC(═S)NRx- or RySC(═O)NRx— end group, with Rx and Ry as defined herein.

The term “hydrazine”, as used herein, describes a —NRx-NRyRw end group or —NRx—NRy— linking group, with Rx, Ry, and Rw as defined herein.

As used herein, the term “epoxide” describes a

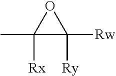

end group or a

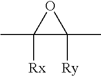

linking group, as these phrases are defined herein, where Rx, Ry and Rw are as defined herein.

For any of the embodiments described herein, a compound, peptide or amino acid as described herein (collectively referred to as “compound” herein) may be in a form of a salt.

As used herein, the salt is a charged species of the parent compound and its counter-ion, which is typically used to modify the solubility characteristics of the parent compound and/or to reduce any significant effect to an organism by the parent compound, while not abrogating the biological activity and properties of the compound. A salt of a compound as described herein can alternatively be formed during the synthesis of the compound, e.g., in the course of isolating the compound from a reaction mixture or re-crystallizing the compound.

In the context of some of the present embodiments, a salt of the compounds described herein may optionally be an acid addition salt and/or a base addition salt.

An acid addition salt comprises at least one basic (e.g., amine and/or guanidinyl) group of the compound which is in a positively charged form (e.g., wherein the basic group is protonated), in combination with at least one counter-ion, derived from the selected acid, that forms a salt. The acid addition salts of the compounds described herein may therefore be complexes formed between one or more basic groups of the compound and one or more equivalents of an acid.

A base addition salt comprises at least one acidic (e.g., carboxylic acid) group of the compound which is in a negatively charged form (e.g., wherein the acidic group is deprotonated), in combination with at least one counter-ion, derived from the selected base, that forms a salt. The base addition salts of the compounds described herein may therefore be complexes formed between one or more acidic groups of the compound and one or more equivalents of a base.

Depending on the stoichiometric proportions between the charged group(s) in the compound and the counter-ion in the salt, the acid additions salts and/or base addition salts can be either mono-addition salts or poly-addition salts.

The phrase “mono-addition salt”, as used herein, refers to a salt in which the stoichiometric ratio between the counter-ion and charged form of the compound is 1:1, such that the addition salt includes one molar equivalent of the counter-ion per one molar equivalent of the compound.

The phrase “poly-addition salt”, as used herein, refers to a salt in which the stoichiometric ratio between the counter-ion and the charged form of the compound is greater than 1:1 and is, for example, 2:1, 3:1, 4:1 and so on, such that the addition salt includes two or more molar equivalents of the counter-ion per one molar equivalent of the compound.

An example, without limitation, of a salt would be an ammonium cation or guanidinium cation and an acid addition salt thereof, and/or a carboxylate anion and a base addition salt thereof.

The base addition salts may include a cation counter-ion such as sodium, potassium, ammonium, calcium, magnesium and the like, that forms a salt.

The acid addition salts may include a variety of organic and inorganic acids, such as, but not limited to, hydrochloric acid which affords a hydrochloric acid addition salt, hydrobromic acid which affords a hydrobromic acid addition salt, acetic acid which affords an acetic acid addition salt, ascorbic acid which affords an ascorbic acid addition salt, benzenesulfonic acid which affords a besylate addition salt, camphorsulfonic acid which affords a camphorsulfonic acid addition salt, citric acid which affords a citric acid addition salt, maleic acid which affords a maleic acid addition salt, malic acid which affords a malic acid addition salt, methanesulfonic acid which affords a methanesulfonic acid (mesylate) addition salt, naphthalenesulfonic acid which affords a naphthalenesulfonic acid addition salt, oxalic acid which affords an oxalic acid addition salt, phosphoric acid which affords a phosphoric acid addition salt, toluenesulfonic acid which affords a p-toluenesulfonic acid addition salt, succinic acid which affords a succinic acid addition salt, sulfuric acid which affords a sulfuric acid addition salt, tartaric acid which affords a tartaric acid addition salt and trifluoroacetic acid which affords a trifluoroacetic acid addition salt. Each of these acid addition salts can be either a mono-addition salt or a poly-addition salt, as these terms are defined herein.

Further, each of the compounds described herein, including the salts thereof, can be in a form of a solvate or a hydrate thereof.

The term “solvate” refers to a complex of variable stoichiometry (e.g., di-, tri-, tetra-, penta-, hexa-, and so on), which is formed by a solute (the heterocyclic compounds described herein) and a solvent, whereby the solvent does not interfere with the biological activity of the solute.

The term “hydrate” refers to a solvate, as defined hereinabove, where the solvent is water.

The compounds described herein can be used as polymorphs and the present embodiments further encompass any isomorph of the compounds and any combination thereof.

The compounds and structures described herein encompass any stereoisomer, including enantiomers and diastereomers, of the compounds described herein, unless a particular stereoisomer is specifically indicated.

As used herein, the term “enantiomer” refers to a stereoisomer of a compound that is superposable with respect to its counterpart only by a complete inversion/reflection (mirror image) of each other. Enantiomers are said to have “handedness” since they refer to each other like the right and left hand. Enantiomers have identical chemical and physical properties except when present in an environment which by itself has handedness, such as all living systems. In the context of the present embodiments, a compound may exhibit one or more chiral centers, each of which exhibiting an (R) or an (S) configuration and any combination, and compounds according to some embodiments of the present invention, can have any their chiral centers exhibit an (R) or an (S) configuration.

The term “diastereomers”, as used herein, refers to stereoisomers that are not enantiomers to one another. Diastereomerism occurs when two or more stereoisomers of a compound have different configurations at one or more, but not all of the equivalent (related) stereocenters and are not mirror images of each other. When two diastereoisomers differ from each other at only one stereocenter they are epimers. Each stereo-center (chiral center) gives rise to two different configurations and thus to two different stereoisomers. In the context of the present invention, embodiments of the present invention encompass compounds with multiple chiral centers that occur in any combination of stereo-configuration, namely any diastereomer.

Herein, the term “peptide” refers to a substance comprising at least 2 amino acid residues linked by peptide bonds or analogs thereof (as described herein below), and optionally only by peptide bonds per se. In some embodiments, the peptide comprises from 2 to 10, preferably from 2 to 6, or from 2 to 4, amino acid residues or analogs thereof.

The term “peptide” encompasses native peptides (e.g., degradation products, synthetically synthesized peptides and/or recombinant peptides), including, without limitation, native proteins, fragments of native proteins and homologs of native proteins and/or fragments thereof; as well as peptidomimetics (typically, synthetically synthesized peptides) and peptoids and semipeptoids which are peptide analogs, which may have, for example, modifications rendering the peptides more stable while in a body or more capable of penetrating into cells. Such modifications include, but are not limited to N-terminus modification, C-terminus modification, peptide bond modification, backbone modifications, and residue modification. Methods for preparing peptidomimetic compounds are well known in the art and are specified, for example, in Quantitative Drug Design, C. A. Ramsden G d., Chapter 17.2, F. Choplin Pergamon Press (1992), which is incorporated by reference as if fully set forth herein. Further details in this respect are provided herein below.

Peptide bonds (—CO—NH—) within the peptide may be substituted, for example, by N-methylated amide bonds (—N(CH3)—CO—), ester bonds (—C(═O)—O—), ketomethylene bonds (—CO—CH2—), sulfinylmethylene bonds (—S(═O)—CH2—), α-aza bonds (—NH—N(R)—CO—), wherein R is any alkyl (e.g., methyl), amine bonds (—CH2—NH—), sulfide bonds (˜CH2-S—), ethylene bonds (˜CH2-CH2—), hydroxyethylene bonds (—CH(OH)—CH2—), thioamide bonds (—CS—NH—), olefinic double bonds (—CH═CH—), fluorinated olefinic double bonds (—CF═CH—), retro amide bonds (—NH—CO—), peptide derivatives (—N(R)—CH2—CO—), wherein R is the “normal” side chain, naturally present on the carbon atom.

These modifications can occur at any of the bonds along the peptide chain and even at several (2-3) bonds at the same time.

Natural aromatic amino acids, Trp, Tyr and Phe, may be substituted by non-natural aromatic amino acids such as 1,2,3,4-tetrahydroisoquinoline-3-carboxylic acid (Tic), naphthylalanine, ring-methylated derivatives of Phe, halogenated derivatives of Phe or O-methyl-Tyr.

The peptides of some embodiments of the invention may also include one or more modified amino acids or one or more non-amino acid monomers (e.g. fatty acids, complex carbohydrates etc.).

The term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids.

Tables 1 and 2 below list naturally occurring amino acids (Table 1), and non-conventional or modified amino acids (e.g., synthetic, Table 2) which can be used with some embodiments of the invention.

TABLE 1

| Amino | Three-Letter | One-letter |
|---|---|---|
| Acid | Abbreviation | Symbol |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic Acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Any amino acid as above | Xaa | X |

TABLE 2

| Non-conventional | | Non-conventional | |
|---|---|---|---|
| amino acid | Code | amino acid | Code |
| ornithine | Orn | hydroxyproline | Hyp |
| α-aminobutyric acid | Abu | aminonorbornyl- | Norb |
| | | carboxylate | |
| D-alanine | Dala | aminocyclopropane- | Cpro |
| | | carboxylate | |
| D-arginine | Darg | N-(3- | Narg |
| | | guanidinopropyl)glycine | |
| D-asparagine | Dasn | N-(carbamylmethyl)glycine | Nasn |
| D-aspartic acid | Dasp | N-(carboxymethyl)glycine | Nasp |
| D-cysteine | Dcys | N-(thiomethyl)glycine | Ncys |
| D-glutamine | Dgln | N-(2-carbamylethyl)glycine | Ngln |
| D-glutamic acid | Dglu | N-(2-carboxyethyl)glycine | Nglu |
| D-histidine | Dhis | N-(imidazolylethyl)glycine | Nhis |
| D-isoleucine | Dile | N-(1-methylpropyl)glycine | Nile |
| D-leucine | Dleu | N-(2-methylpropyl)glycine | Nleu |
| D-lysine | Dlys | N-(4-aminobutyl)glycine | Nlys |
| D-methionine | Dmet | N-(2-methylthioethyl)glycine | Nmet |
| D-ornithine | Dorn | N-(3-aminopropyl)glycine | Norn |
| D-phenylalanine | Dphe | N-benzylglycine | Nphe |
| D-proline | Dpro | N-(hydroxymethyl)glycine | Nser |
| D-serine | Dser | N-(1-hydroxyethyl)glycine | Nthr |
| D-threonine | Dthr | N-(3-indolylethyl) glycine | Nhtrp |
| D-tryptophan | Dtrp | N-(p-hydroxyphenyl)glycine | Ntyr |
| D-tyrosine | Dtyr | N-(1-methylethyl)glycine | Nval |
| D-valine | Dval | N-methylglycine | Nmgly |
| D-N-methylalanine | Dnmala | L-N-methylalanine | Nmala |
| D-N-methylarginine | Dnmarg | L-N-methylarginine | Nmarg |
| D-N-methylasparagine | Dnmasn | L-N-methylasparagine | Nmasn |
| D-N-methylasparatate | Dnmasp | L-N-methylaspartic acid | Nmasp |
| D-N-methylcysteine | Dnmcys | L-N-methylcysteine | Nmcys |
| D-N-methylglutamine | Dnmgln | L-N-methylglutamine | Nmgln |
| D-N-methylglutamate | Dnmglu | L-N-methylglutamic acid | Nmglu |
| D-N-methylhistidine | Dnmhis | L-N-methylhistidine | Nmhis |
| D-N-methylisoleucine | Dnmile | L-N-methylisolleucine | Nmile |
| D-N-methylleucine | Dnmleu | L-N-methylleucine | Nmleu |
| D-N-methyllysine | Dnmlys | L-N-methyllysine | Nmlys |
| D-N-methylmethionine | Dnmmet | L-N-methylmethionine | Nmmet |
| D-N-methylornithine | Dnmorn | L-N-methylornithine | Nmorn |
| D-N-methylphenylalanine | Dnmphe | L-N-methylphenylalanine | Nmphe |
| D-N-methylproline | Dnmpro | L-N-methylproline | Nmpro |
| D-N-methylserine | Dnmser | L-N-methylserine | Nmser |
| D-N-methylthreonine | Dnmthr | L-N-methylthreonine | Nmthr |
| D-N-methyltryptophan | Dnmtrp | L-N-methyltryptophan | Nmtrp |
| D-N-methyltyrosine | Dnmtyr | L-N-methyltyrosine | Nmtyr |
| D-N-methylvaline | Dnmval | L-N-methylvaline | Nmval |
| L-norleucine | Nle | L-N-methylnorleucine | Nmnle |
| L-norvaline | Nva | L-N-methylnorvaline | Nmnva |
| L-ethylglycine | Etg | L-N-methyl-ethylglycine | Nmetg |
| L-t-butylglycine | Tbug | L-N-methyl-t-butylglycine | Nmtbug |
| L-homophenylalanine | Hphe | L-N-methyl- | Nmhphe |
| | | homophenylalanine | |
| α-naphthylalanine | Anap | N-methyl-α-naphthylalanine | Nmanap |
| penicillamine | Pen | N-methylpenicillamine | Nmpen |
| γ-aminobutyric acid | Gabu | N-methyl-γ-aminobutyrate | Nmgabu |
| cyclohexylalanine | Chexa | N-methyl-cyclohexylalanine | Nmchexa |
| cyclopentylalanine | Cpen | N-methyl-cyclopentylalanine | Nmcpen |
| α-amino-α-methylbutyrate | Aabu | N-methyl-α-amino-α- | Nmaabu |
| | | methylbutyrate | |
| α-aminoisobutyric acid | Aib | N-methyl-α- | Nmaib |
| | | aminoisobutyrate | |
| D-α-methylarginine | Dmarg | L-α-methylarginine | Marg |
| D-α-methylasparagine | Dmasn | L-α-methylasparagine | Masn |
| D-α-methylaspartate | Dmasp | L-α-methylaspartate | Masp |
| D-α-methylcysteine | Dmcys | L-α-methylcysteine | Mcys |
| D-α-methylglutamine | Dmgln | L-α-methylglutamine | Mgln |
| D-α-methyl glutamic acid | Dmglu | L-α-methylglutamate | Mglu |
| D-α-methylhistidine | Dmhis | L-α-methylhistidine | Mhis |
| D-α-methylisoleucine | Dmile | L-α-methylisoleucine | Mile |
| D-α-methylleucine | Dmleu | L-α-methylleucine | Mleu |
| D-α-methyllysine | Dmlys | L-α-methyllysine | Mlys |
| D-α-methylmethionine | Dmmet | L-α-methylmethionine | Mmet |
| D-α-methylornithine | Dmorn | L-α-methylornithine | Morn |
| D-α-methylphenylalanine | Dmphe | L-α-methylphenylalanine | Mphe |
| D-α-methylproline | Dmpro | L-α-methylproline | Mpro |
| D-α-methylserine | Dmser | L-α-methylserine | Mser |
| D-α-methylthreonine | Dmthr | L-α-methylthreonine | Mthr |
| D-α-methyltryptophan | Dmtrp | L-α-methyltryptophan | Mtrp |
| D-α-methyltyrosine | Dmtyr | L-α-methyltyrosine | Mtyr |
| D-α-methylvaline | Dmval | L-α-methylvaline | Mval |
| N-cyclobutylglycine | Ncbut | L-α-methylnorvaline | Mnva |
| N-cycloheptylglycine | Nchep | L-α-methylethylglycine | Metg |
| N-cyclohexylglycine | Nchex | L-α-methyl-t-butylglycine | Mtbug |
| N-cyclodecylglycine | Ncdec | L-α-methyl- | Mhphe |
| | | homophenylalanine | |
| N-cyclododecylglycine | Ncdod | α-methyl-α-naphthylalanine | Manap |
| N-cyclooctylglycine | Ncoct | α-methylpenicillamine | Mpen |
| N-cyclopropylglycine | Ncpro | α-methyl-γ-aminobutyrate | Mgabu |
| N-cycloundecylglycine | Ncund | α-methyl-cyclohexylalanine | Mchexa |
| N-(2-aminoethyl)glycine | Naeg | α-methyl-cyclopentylalanine | Mcpen |
| N-(2,2- | Nbhm | N-(N-(2,2-diphenylethyl) | Nnbhm |
| diphenylethyl)glycine | | carbamylmethyl-glycine | |
| N-(3,3- | Nbhe | N-(N-(3,3-diphenylpropyl) | Nnbhe |
| diphenylpropyl)glycine | | carbamylmethyl-glycine | |
| 1-carboxy-1-(2,2-diphenyl | Nmbc | 1,2,3,4- | Tic |
| ethylamino)cyclopropane | | tetrahydroisoquinoline-3- | |
| | | carboxylic acid | |
| phosphoserine | pSer | phosphothreonine | pThr |
| phosphotyrosine | pTyr | O-methyl-tyrosine | |
| 2-aminoadipic acid | | hydroxylysine | |

The peptides of some embodiments of the invention are preferably utilized in a linear form, although it will be appreciated that in cases where cyclization does not severely interfere with peptide characteristics, cyclic forms of the peptide can also be utilized.

Since the present peptides are preferably utilized in therapeutics or diagnostics which require the peptides to be in soluble form, the peptides of some embodiments of the invention preferably include one or more non-natural or natural polar amino acids, including but not limited to serine and threonine which are capable of increasing peptide solubility due to their hydroxyl-containing side chain.

The peptides of some embodiments of the invention may be synthesized by any techniques that are known to those skilled in the art of peptide synthesis. For solid phase peptide synthesis, a summary of the many techniques may be found in J. M. Stewart and J. D. Young, Solid Phase Peptide Synthesis, W. H. Freeman Co. (San Francisco), 1963 and J. Meienhofer, Hormonal Proteins and Peptides, vol. 2, p. 46, Academic Press (New York), 1973. For classical solution synthesis see G. Schroder and K. Lupke, The Peptides, vol. 1, Academic Press (New York), 1965.

In general, these methods comprise the sequential addition of one or more amino acids or suitably protected amino acids to a growing peptide chain. Normally, either the amino or carboxyl group of the first amino acid is protected by a suitable protecting group. The protected or derivatized amino acid can then either be attached to an inert solid support or utilized in solution by adding the next amino acid in the sequence having the complimentary (amino or carboxyl) group suitably protected, under conditions suitable for forming the amide linkage. The protecting group is then removed from this newly added amino acid residue and the next amino acid (suitably protected) is then added, and so forth. After all the desired amino acids have been linked in the proper sequence, any remaining protecting groups (and any solid support) are removed sequentially or concurrently, to afford the final peptide compound. By simple modification of this general procedure, it is possible to add more than one amino acid at a time to a growing chain, for example, by coupling (under conditions which do not racemize chiral centers) a protected tripeptide with a properly protected dipeptide to form, after deprotection, a pentapeptide and so forth. Further description of peptide synthesis is disclosed in U.S. Pat. No. 6,472,505.

A preferred method of preparing the peptide compounds of some embodiments of the invention involves solid phase peptide synthesis.

Large scale peptide synthesis is described by Andersson Biopolymers 2000; 55(3):227-50.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Materials and Methods

### Materials:

Fluorenylmethyloxycarbonyl-diphenylalanine (FmocFF) was obtained from Bachem (Bubendorf, Switzerland).

Fluorenylmethoxycarbonyl-Leucine-Leucine (FmocLL) was obtained from GL Biochem (Shanghai, China).

Dimethyl sulfoxide ReagentPlus®≥99.5%, Methyl viologen dichloride hydrate 98%, and sodium dithionite, technical grade 85% were purchased from Sigma-Aldrich (St. Louis, MO).

Tris(hydroxymethyl)aminomethane (Tris) was purchased from Bio-Lab Ltd (Jerusalem, Israel).

SIGRACELL® GFD 4.65 EA carbon felt (stated thickness of 4.6 millimeters, density of 0.09 grams/cm³, porosity of 94%, BET surface area of 0.4 m²/gram, longitudinal electrical resistivity of <5 Ωmm, and transverse electrical resistivity of <3 Ωmm) was purchased from SGL Carbon (Meitingen, Germany). The diameter of individual carbon fibers was measured in-house in SEM images as approximately 9 μm.

[FeFe] Hydrogenase, Fe Superoxide Dismutase and Ferredoxin-NADP+ Reductase enzymes were expressed and purified in-house, according to published protocols (Ben-Zvi et al., International Journal of Hydrogen Energy, 2016, Vol. 41;, Marco et al., Biochim. Biophys. Acta—Bioenerg., 2019, 1860, 689-698; and Yacoby et al., PLOS ONE 7(4): e35886).

Rabbit polyclonal Chlamydomonas reinhardtii HydA1/2, SOD and FNR primary antibodies were purchased from Agrisera (Vännäs, Sweden).

Cye-5 NHS ester was purchased from Lumiprobe (Hong Kong, China).

### Electrode Preparation:

Electrode fabrication was performed in an anaerobic chamber (3.5% H₂ balance N₂, Coy Laboratories, USA). Tris-HCl pH 7.2 buffered solution was supplemented with 1 mM sodium dithionite, 2 mM MV and HydA enzyme, while peptide hydrogelator powder was dissolved in DMSO to a concentration of 100 mg/mL. The two solutions were then mixed and immediately soaked on 2×1 cm carbon felt, followed by a 2-hours gelation period. For FmocFF electrodes, a final concentration of 35% DMSO and 80 mM Tris-HCl pH 7.2 was used to allow sufficient soaking time due to rapid gelation in buffered solution. FmocLL electrodes were prepared at a final concentration of 10% DMSO, 80 mM Tris-HCl pH 7.2. For solution-soaked electrodes, only 80 mM Tris-HCl pH 7.2 buffer was used. Unless stated otherwise, all electrodes contained 200 micrograms HydA enzyme, while 5 mg peptide hydrogelator was used in either FmocFF or FmocLL electrode.

### Electrochemical Measurements:

Cyclic voltammetry and chronoamperometry were performed using a MultiPalmSens4 (Palm Sense, The Netherlands) potentiostat with three-electrode configuration cells. All experiments were carried out in a custom-made 100 mL electrochemical cell fitted with a sample valve for headspace purging and measurements. The electrolytic cell was filled with 50 mL electrolyte (100 mM Tris HCl pH 7.2, 100 mM KCl) at room temperature. A carbon felt working electrode was threaded onto a platinum wire in an anaerobic environment, and transferred to the electrolytic cell. RE-1S Ag/AgCl electrode (ALS Co., Japan) and platinum mesh were used as the reference and counter electrodes, respectively. All electrodes were wired to the top stopper of the electrochemical cell while submerged in electrolyte solution. Prior to experiment onset, the cell headspace was purged with argon gas for 10 minutes. Scan rate for cyclic voltammetry was 50 mV/second in a range of −0.8 to 0.4V Vs SHE. The potential for chronoamperometry was set at −0.6 V Vs SHE for a duration of 18 hours. H₂ gas was measured by sampling the cell headspace using Hewlett-Packard 5890 Series II gas chromatograph (Agilent Technologies, USA).

### HydA Enzymatic Activity Measurement:

Carbon felt electrodes were prepared as described hereinabove. In addition, samples were subjected to 1 hour washing in 50 mL electrolyte per electrode, to remove DMSO traces. Following washing, fresh samples were assayed immediately while other tested samples were incubated for 18 hours under different conditions: aged samples were removed to dry in 7 mL septum-sealed serum glass vials (Wheaton), immersed samples were left in the electrolyte without further procedures, and electrochemically-activated samples were subjected to a constant potential of −0.6 V Vs SHE in an electrolytic cell. HydA activity determination was then conducted by placing the carbon felt electrodes in 7 mL septum-sealed serum glass vials under anaerobic conditions, while purging with argon gas for 10 minutes, and supplementing the vials with 1 mL activity buffer (100 mM Tris-HCl pH 7.2, 1M NaCl, 20 mM sodium dithionite, and 10 mM MV). The vials were then incubated at 50° C. in a water bath, while 500 microliters of headspace gas samples were drawn at 6-minute intervals. The concentration of H₂ in the vial headspace was measured using a Hewlett-Packard 5890 Series II gas chromatograph (Agilent Technologies, USA). The residual activity of HydA was determined by comparing the samples to the corresponding fresh control.

### Immunoblot:

The electrolyte from the electrolytic cell was collected following 18-hour chronoamperometry and concentrated 100-fold using Amicon® Ultra 15 mL centrifugal device, with a cutoff of 10,000 Dalton (Merc, USA). Equal volumes of electrolyte were incubated for 10 minutes at 72° C. with Bolt LDS Sample Buffer (Invitrogen, USA). Samples were then loaded on 4-12 Bis-Tris Plus PAGE gels© (Invitrogen, USA) and analyzed by immunoblotting using iBind blotter and its specific blocking reagents© (Invitrogen, USA). Rabbit polyclonal HydA1/2, SOD, and FNR antibodies were used as primary antibodies. Membrane images were taken using an Amersham ImageQuant 800 station (Cytiva, USA).

### Rheology:

Rheology measurements were carried out using an AR-G2 controlled-stress rheometer (TA Instruments, USA). In order to determine the linear viscoelastic region, oscillatory strain (0.01-100%) and frequency sweep (0.01-100 Hz) tests were performed in parallel plate geometry. The hydrogels were prepared by pipetting Tris buffer (100 mM, pH 7.2) onto FmocFF solution in DMSO, reaching final FmocFF concentrations of 1-5 milligrams mL⁻¹ and final DMSO concentration of 35%, and immediately dropping 250 microliters of the mixture onto the rheometer plate. The geometry was immediately set at a gap size of 600 micrometers, and a 2-hour soak time was allowed prior to measurement onset. Time sweep oscillatory tests were performed for 35 minutes at a constant frequency of 1 Hz and strain of 0.1% to determine G′ and G″, the storage and loss moduli, respectively. All measurements were conducted at room temperature.

### Scanning Electron Microscopy:

Carbon felt electrodes were prepared as described above followed by 1 hour washing with 50 mL Tris-HCl buffer (100 mM pH 7.2) per electrode to remove DMSO traces. To obtain cross section views of the electrode interior, samples were flash frozen in liquid nitrogen before being fragmented. Samples were freeze-dried, and Au sputter coating of dried samples was performed prior to imaging in a JEOL JSM-IT 100 scanning electron microscope (JEOL, Japan) operating at 20 kV.

### Confocal Microscopy:

Proteins were diluted in phosphate buffer (100 mM, pH 8) to 1 mg mL⁻¹. Cy5 NHS ester was dissolved in DMF and added to the protein solution at 3:1 molar ratio, while keeping the final concentration of DMF at 10%. The solution was shaken overnight at 4° C., and then DMF, buffer and excess dye were washed with Tris-HCl buffer (100 mM, pH 7.2) using a 10,000 Dalton cutoff Amicon® Ultra 15 mL centrifugal device. Samples of carbon felt electrode containing stained protein were prepared as described above. Hydrogels samples containing stained proteins were prepared in a similar manner but without carbon felt. Imaging was performed using ZEISS LSM 900 confocal microscope (ZEISS, Germany).

### Example 1

Electrode preparation was performed in an anaerobic chamber, and is schematically illustrated in FIG. 1. Tris-HCl pH 7.2 buffered solution was supplemented with methyl viologen (MV) and HydA enzyme, and mixed with a DMSO solution of the peptide hydrogelator (of a plurality of peptides that form a self-assembled fibrillar structure). Rapid initiation of gelation necessitated immediate soaking of the mixture onto the carbon felt. Self-assembly was allowed to occur for 2 hours. The electrodes were then tethered on a platinum wire and placed in an electrochemical cell (see, for example, FIG. 1). Electrodes were prepared using self-assembled Fmoc-Phenylalanine-Phenylalanine (FmocFF) hydrogels or Fmoc-Leucine-Leucine (FmocLL), the structures of which are shown in FIG. 2.

The cyclic voltammograms are presented in FIGS. 3A-D for the following working electrodes: FmocFF hydrogel supplemented with HydA and MV soaked on a carbon felt electrode (FIG. 3A), FmocFF hydrogel supplemented with MV soaked on carbon felt electrode (FIG. 3B), carbon felt soaked with FmocFF and active HydA without MV (FIG. 3C), and carbon felt soaked with only FmocFF hydrogel with no other additives (FIG. 3D).

As can be seen from the cyclic voltammetry measurements represented in FIG. 3A, the combination of FmocFF hydrogel with both MV and HydA produced a strong reduction peak, reaching a current of 3.5 mA at −0.54 V, attributed to the reduction of MV⁺² (MV^{ox}) to MV⁺ (MV^{red}). An oxidation peak was detected at −0.33 V as the MV^{red} pool was oxidized back to MV^{ox}, followed by a sustained current of 1 mA between −0.2 V to 0.4 V. This observation may be due to H₂ gas, produced by HydA during the reduction phase, being oxidized by the reverse reaction of the enzyme, which reduces MV^{ox} to MV^{red}. Subsequently, MV^{red} was oxidized at the electrode, resulting in the measured current between −0.2 V to 0.4 V.

As can be seen in FIG. 3B, the FmocFF hydrogel with MV alone yielded a similarly cyclic voltammogram, but without any detectable current following MV oxidation at −0.33V.

These observations demonstrate the mobility of MV in the FmocFF hydrogel as it readily carried electrons to and from the biocatalyst, as schematically shown in FIG. 3E. As can be seen in FIG. 3C, carbon felt electrode soaked with FmocFF and active HydA enzyme, without the presence of MV did not produce a notable current, showing only a minor reduction peak at −0.45V. This is in line with the known redox potential of the HydA active site (Silakov et al., Biochemistry, 2009, 48, 7780-7786). These data indicate that while some direct electron transfer does occur, most of the encapsulated enzyme is too distant from the electrode surface, and that an electron transfer mediator is required for the utilization of the electrode's full potential. As can be seen in FIG. 3D, carbon felt electrode soaked with FmocFF hydrogel alone produced no peaks throughout the range of −0.8 V to 0.4 V vs. a standard hydrogen electrode (SHE).

### Example 2

To Different concentrations of the enzyme were loaded into the hydrogels made of FmocFF or FmocLL and associated with a carbon felt electrode, while the size of the carbon felt electrode and the hydrogel volume were kept constant. The samples were prepared in an anaerobic environment and placed as a working electrode in a 3-electrodes electrochemical cell as described herein. Chronoamperometry was recorded over-night at −0.6 V SHE, and the cell headspace was sampled to quantify the total H₂ production following 18 hours of potential application. Data was compared to electrochemically measurements performed with carbon felt electrode to which the enzyme was soaked directly from solution (with no hydrogel).

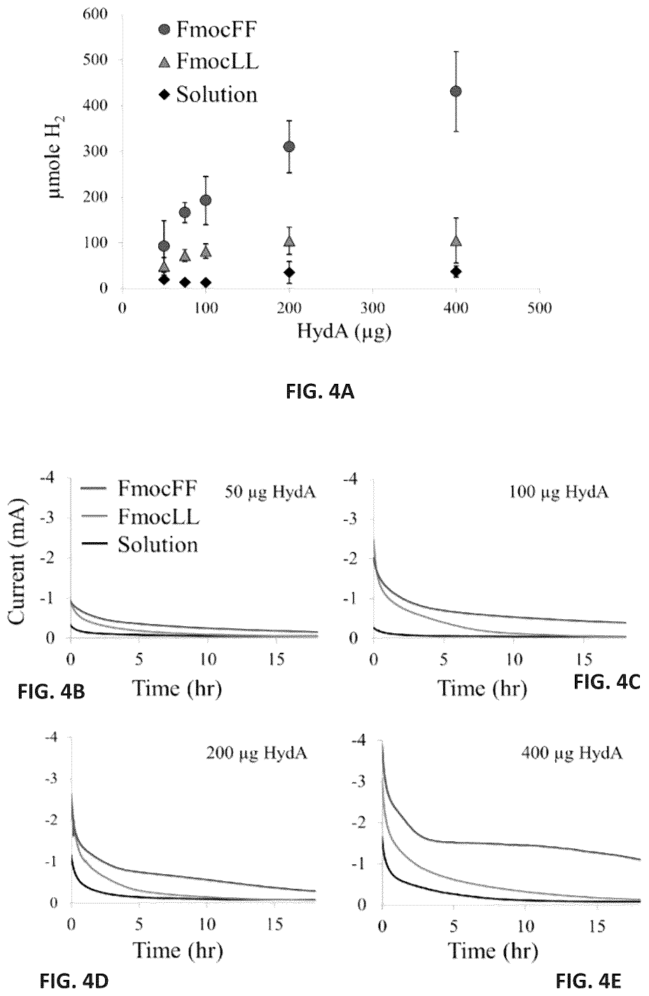

FIGS. 4A-E present the obtained data. FIG. 4A presents a graph showing the accumulated H₂ produced overnight in each of the tested electrochemical setups. FIGS. 4B-E present the corresponding chronamperometries of overnight electrochemical assays performed with FmocFF, FmocLL, and solution-soaked carbon felt electrodes for 50 micrograms HydA (FIG. 4A), 100 micrograms HydA (FIG. 4C), 200 micrograms HydA (FIG. 4D), and 400 micrograms HydA (FIG. 4E).

As shown in FIG. 4A, it was observed that increasing the concentrations of HydA in carbon felt electrodes, in the absence of a hydrogel matrix, failed to result in increased H₂ production, presumably due to enzyme diffusion out of the electrode. Encapsulation of HydA in the FmocLL-soaked electrode produced a mild improvement in total H₂ production, however, not in a concentration-dependent manner. In contrast, H₂ production was in direct correlation to the concentrations of HydA encapsulated in the FmocFF-soaked carbon felt electrode. The accumulated H₂ increased from 90 micromole to 430 micromole as the quantity of HydA enzyme was raised from 50 micrograms to 400 micrograms.

As shown in FIG. 4B-E, the recorded chronoamperometry for all enzyme concentrations showed a similar trend, with solution-soaked electrodes exhibiting a low current immediately upon voltage application, which continued to decline until voltage termination. For the hydrogel-soaked carbon felt electrodes, the currents recorded for the FmocLL and FmocFF samples were similar in the first 10 minutes, with generally higher currents for higher enzyme concentrations. As the measurements continued, a decay of the current was observed in all samples. However, the decay was observed to be much steeper in the FmocLL-containing electrodes, where the reaction practically came to a halt after about 10 hours. In contrast, the current decay observed in the FmocFF samples was milder, with sufficient current to produce visible H₂ bubbling from the electrode even after 18 hours.

By integrating the area under the current curve for the total charge (FIGS. 4B-E) and considering the moles of H₂ that were produced (FIG. 4A), the faradaic efficiency was calculated and found to be approximately 80-90% for all the tested conditions. This high efficiency can be attributed to the high specificity of the enzymatic reaction. Still, the relatively low faradaic loss indicates some unwanted side reactions, for example O₂ reduction into O²⁻ by MV^{red}. O₂ may be introduced into the system via minor air leaks. The relatively stable enzymatic activity in the FmocFF hydrogel, as observed by chronoamperometry, suggests a strong enzyme encaging ability. Hence, soaking the carbon felt within the FmocFF hydrogel allowed the retention of a higher concentration of the enzyme in proximity to the electrode. Indeed, H₂ production yields in FmocFF hydrogel increase with enzyme concentration due to higher overall enzymatic activity. In contrast, the complete decay of the activity in the FmocLL hydrogel suggests a weak retention ability. This is further supported by the inability to achieve higher H₂ production at a high enzyme concentration (FIG. 4A), likely since excess enzyme diffuses out of the electrode. The difference between the two peptide hydrogels is intriguing, as both have similar chemical and assembly properties, form hydrogels with nano-fibrillar structure, with FmocLL shown to increase the stability of a [FeFe] hydrogenase model compound (Frederix et al., Dalt. Trans., 2012, 41, 13112-13119).

### Example 3

To investigate the mode of enzyme entrapment by the peptide hydrogels within the carbon felt electrode, confocal microscopy analysis was performed. Carbon felt electrodes were prepared using HydA covalently linked to the fluorescent dye Cy5.

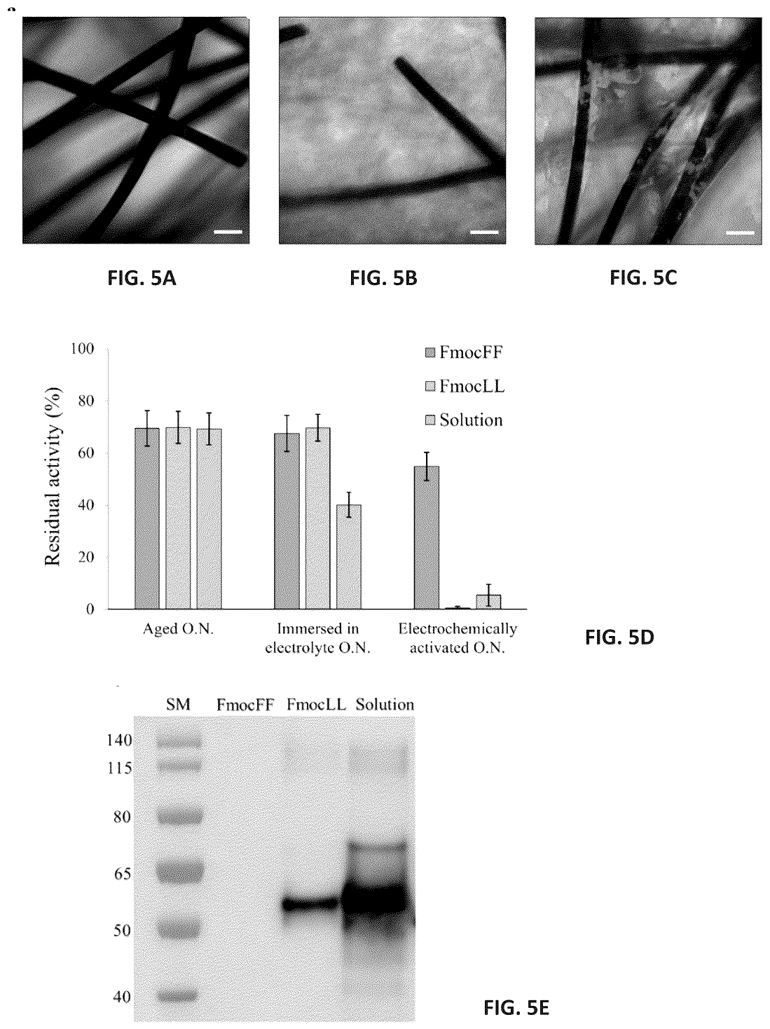

FIGS. 5A-C present overlayed confocal images of fluorescent Cy5-stained HydA enzyme, when soaked from solution on carbon felt electrode (FIG. 5A), of HydA encapsulated in FmocLL hydrogel (FIG. 5B) and HydA encapsulated in FmocFF hydrogel (FIG. 5C), as visualized via brightfield.

As shown in FIGS. 5A-C, neither pristine FmocFF nor FmocLL fibrils displayed fluorescence in the tested wavelengths; therefore, detected fluorescence was attributed to the dyed enzyme. While the enzyme encapsulated in the FmocLL hydrogel appeared to be arranged into clumps in between the carbon fibers (FIG. 5B), the enzyme encapsulated in FmocFF was concentrated directly on the carbon felt fibers (FIG. 5C). This arrangement holds potential in overcoming limitations of mediator diffusion due to the short distance between the enzyme and the electrode surface. Inspecting the solution-soaked carbon felt electrode revealed no fluorescence signal, most likely due to the enzyme not being concentrated in a certain location, but rather evenly distributed throughout the sample (FIG. 5A). This difference in morphology may infer a difference in the ability to retain the enzyme within the carbon felt electrode.

### Example 4

To elucidate the decline in H₂ production over time, it was tested whether the enzyme was slowly inactivated or rather diffusing out of the carbon felt. A chemical assay was performed measuring the residual activity of HydA soaked in the carbon felt electrode, with and without hydrogel encapsulation. Samples of 75 μg (micrograms) HydA were used in: i) Solution-soaked carbon felt electrode ii) FmocLL hydrogel-soaked carbon felt electrode, and iii) FmocFF hydrogel-soaked carbon felt electrode. Fresh samples were assayed immediately to establish the activity baseline for each group. To investigate a possible aging effect, samples were placed in sealed vials overnight and then the innate loss of activity, if any, was measured. To investigate the possible role of electrophoresis, another group of samples was subjected to steady electrical current during an overnight electrochemical H₂ production experiment

To test for passive diffusion of the enzyme out of the electrode and into the electrolyte, the samples were immersed in an electrolyte solution overnight.

FIG. 5D presents a bar graph presenting the residual activity of HydA soaked on carbon felt electrode in either overnight (O·N) aging, immersed in electrolyte solution O·N, or electrochemically activated O·N, for HydA encapsulated in FmocLL hydrogel, HydA encapsulated in FmocFF hydrogel, and HydA in solution

As shown in FIG. 5D, overnight aging resulted in a 30% loss of activity regardless of the encapsulation method, suggesting that the enzyme is stable in the peptide hydrogels as no specific loss of activity was observed, and that the enzyme stability cannot account for the difference observed in the electrochemical assay. When immersed in electrolyte solution, both hydrogels showed the same activity as the aged samples, while the solution-soaked electrodes lost an additional 30% of activity (60% in total). In samples subjected to steady electrical current during an overnight electrochemical H₂ production experiment, it was observed that while the residual enzymatic activities of both FmocLL and solution samples were extremely low, the samples encapsulated in FmocFF retained 55% of the HydA activity, compared to fresh samples. These observations imply that both hydrogels successfully prevented the enzyme from passively diffusing out. However, a significant difference between the two hydrogels was observed when the samples were subjected to overnight electrochemical activation. These results are in accordance with the chronoamperometry analysis shown in FIG. 4A and the corresponding H₂ produced.

The loss of HydA activity in the FmocLL hydrogel can be attributed to either inactivation of the encapsulated enzyme or electrophoresis of active protein out of the hydrogel. To distinguish between these options, the electrolyte solution from the electrochemical cells was collected, concentrated, and analyzed by immunoblot assay using specific anti-HydA antibodies to detect leaked enzyme. FIG. 5E presents an immunoblot of HydA in electrolyte collected after O·N. electrochemical activation in FmocFF hydrogel, FmocLL hydrogel or solution.

As shown in FIG. 5E, a notable protein band detected using the electrolyte sample from the cell comprising either solution-soaked electrode or FmocLL hydrogel electrode, indicating high levels of leaked protein. No protein was detected in the electrolyte surrounding the FmocFF-soaked electrode. This implies that protein electrophoresis is the prominent factor limiting H₂ production in both the solution and FmocLL hydrogel samples. Thus, the retention of HydA in the FmocFF hydrogel, even under electric field, presumably supports the prolonged function of the hydrogel-soaked electrode.

### Example 5

In order to understand whether the formation of a gel is a prerequisite for encapsulation and activity of HydA in the electrochemical system, carbon felt electrodes were soaked with FmocFF at a concentration range of 1-5 mg/mL.

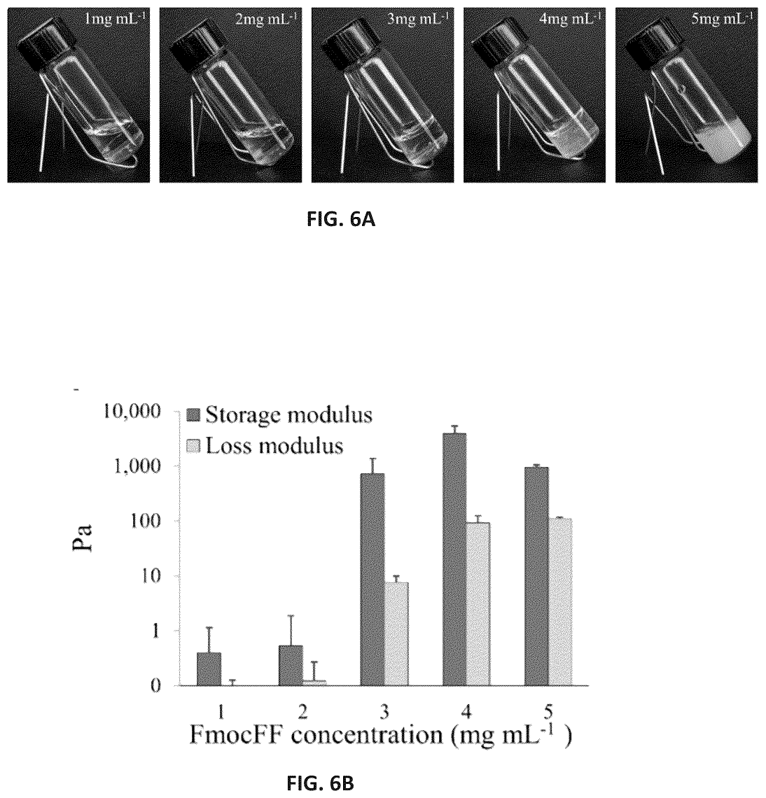

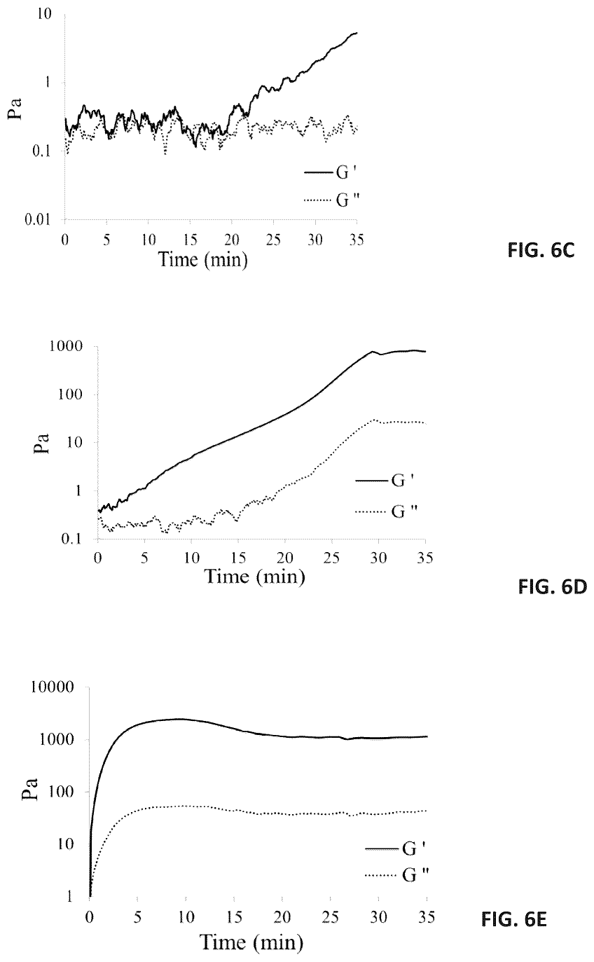

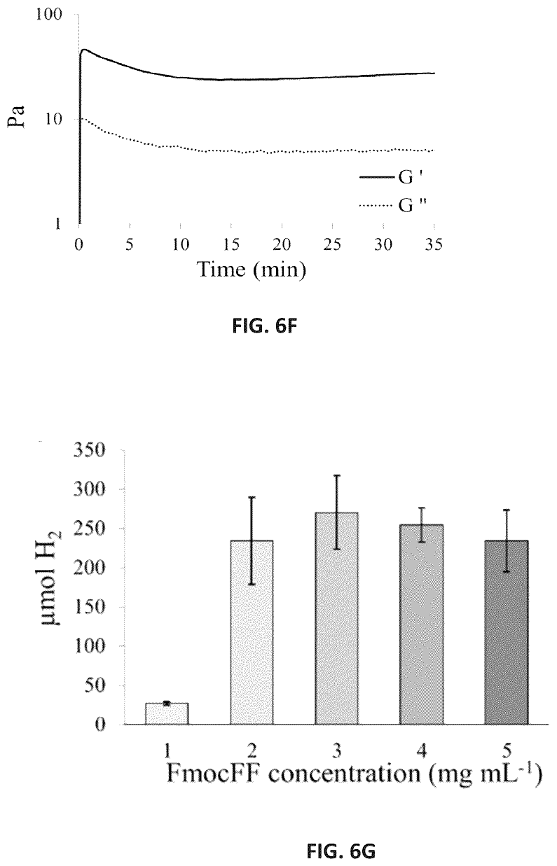

FIGS. 6A-B present images of tilted vials of FmocFF at concentrations of 1, 2, 3, 4 and 5 mg/mL (FIG. 6A) and a graph showing storage modulus and loss modulus of FmocFF at concentrations of 1, 2, 3, 4 and 5 mg/mL (FIG. 6B).

As shown in FIG. 6A, a liquid suspension of self-assembled fibrils, rather than a self-supporting hydrogel, was formed at 1-2 mg/mg peptide concentration. Concentrations of 3-5 mg/mL produced a stable gel, with visible increasing turbidity, correlating to the increase in peptide concentration. These observations were supported by rheology measurements, as shown in FIG. 6B, where 1-2 mg/mL samples exhibited very low storage and loss moduli, indicating their liquid state. In contrast, a gel state was indicated for the 3-5 mg/mL samples by the higher storage moduli compared to the respective loss moduli.

FIGS. 6C-F present graphs showing storage and loss moduli kinetics of FmocFF at concentrations of 3, 4, 5 and 6 mg/mL, respectively over 35 minutes. As can be seen, increasing FmocFF concentration resulted in shorter gelation times. This gelation kinetics presented a technical difficulty for the fabrication of FmocFF-soaked electrodes with concentrations exceeding 5 mg/mL, as a liquid state of at least 5 to 10 seconds is desired for complete absorption into the carbon felt electrode.

FIG. 6G is a bar graph showing the accumulated H₂ after O·N. electrochemical activation of HydA enzyme, encapsulated in 1, 2, 3, 4 and 5 mg/mL FmocFF and soaked on carbon felt electrode.

As can be seen, while both 1 and 2 mg/mL hydrogel concentrations displayed liquid suspension characteristics, electrochemical H₂ production measurements showed a major difference between them. The 2 mg/mL samples showed higher H₂ production, reaching results similar to the non-liquid hydrogel samples (see, FIG. 6A). Electrochemical H₂ production was not significantly different among the 3-5 mg/mL hydrogel samples, despite the differences in turbidity and mechanical properties (shown e.g., in FIGS. 6A-B). Protein immobilization within a polymer matrix can be the result of physical entrapment of the biomolecule between the dense fibers (Rodriguez-Abetxuko et al., Front. Bioeng. Biotechnol., 2020, 8). If indeed HydA retention is governed by its entanglement in the FmocFF fibril mesh, a less dense matrix is anticipated to permit more protein leakage. Hence, as peptide concentration decreases, a gradual decline in protein retention ability and H₂ production was expected. However, no such gradual decline was observed as peptide concentration was decreased from 5 to 3 mg mL−1, and not even as a liquid suspension of 2 mg/mL. A prominent drop in H₂ production was detected only at 1 mg/mL. It appears that FmocFF retention mechanism is not directly dependent on a self-supporting 3D gel state or its properties, but rather on the presence of a critical concentration of supramolecular structures formation.

### Example 6

To better understand the interaction between HydA and FmocFF supramolecular fibrils, Cy5-labeled HydA was encapsulated in FmocFF hydrogels at concentrations of 0-5 mg/mL, without the presence of a carbon felt electrode. The localization of the enzyme was subsequently determined by fluorescent confocal microscopy.

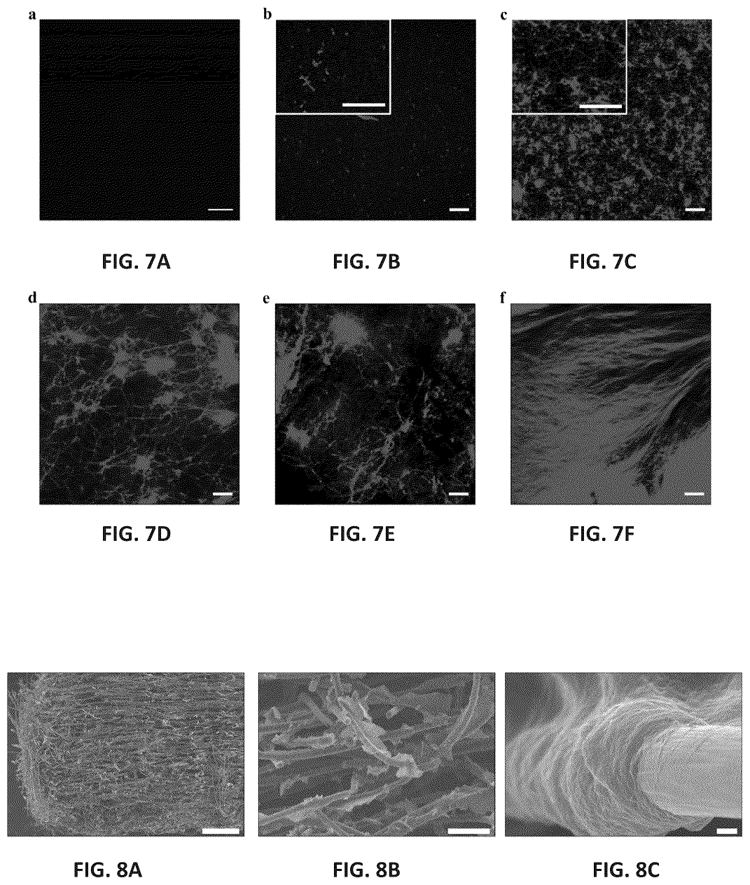

FIGS. 7A-F present confocal images of fluorescent Cy5-stained HydA enzyme with FmocFF at concentrations of 0 mg/mL, 1 mg/mL, 2 mg/mL, 3 mg/mL, 4 mg/mL, and 5 mg/mL, respectively.

As can be seen, no fluorescent signal could be obtained from solution samples (0 mg/mL FmocFF), as the enzyme was not concentrated anywhere in the sample (FIG. 7A). In the presence of 1 mg/mL of FmocFF, some enzyme clusters could be viewed (FIG. 7B). 2 mg/mL of FmocFF produced a distinct fluorescent fibril matrix (FIG. 7C). Further increase in FmocFF concentrations to 3-5 mg/mL resulted in a visible matrix of large fluorescent clusters with inter-connecting fibrils (FIGS. 7D-F, respectively). The visualization of the fluorescent enzyme as a distinct fibril matrix implies direct interaction with the peptide supramolecular structure. These observations are also in line with the H₂ production measured at different FmocFF concentrations (FIG. 6D), further supporting the hypothesis of direct protein-FmocFF interaction. In light of this, the clusters viewed at 1 mg/mL FmocFF can be interpreted as interaction between the protein and small supramolecular structures, insufficient to retain the enzyme on the carbon felt electrode. This interaction may be attributed to physical adsorption, a common immobilization mechanism in which the enzyme adsorbs to the polymer by van der Waals forces, hydrogen bonds, ionic and hydrophobic interactions. However, such immobilization is prone to enzyme leakage due to the relatively weak nature of the forces in play (Jesionowski et al., Adsorption, 2014, 20, 801-821).

Nonetheless, the interaction between HydA and FmocFF appeared to be strong enough, not only to prevent passive diffusion out of the electrode, but also to retain the enzyme in place when potential is applied. When considering physical interactions, π-π and cation-π are among the strongest known, comparable to hydrogen bonds and ion pairing (Dougherty, Acc. Chem. Res., 2013, 885-893). The demonstrated strong retention of HydA in FmocFF compared to FmocLL may stem from the fewer aromatic rings of FmocLL and the consequent lower probability for aromatic interactions (FIG. 2). A difference in the peptides' supramolecular arrangements could also contribute to this phenomenon, as the assembled FmocLL may expose fewer of the peptide's aromatic amine and hydroxyl groups, rendering the assembled fibril less prone to interact with the protein's available residues.

Binding of the protein to peptide fibrils cannot fully account for retention in carbon felt electrodes. To achieve such retention, the enzyme-fibril complex should also attach to the carbon felt electrode and remain inside it throughout the electrochemical activation. It was initially hypothesized that the hydrogels fibril matrix is distributed homogeneously and fills the spaces between the carbon fibers as well as encapsulate HydA in their fibril mesh. While the results obtained with Cy5-labeled HydA encapsulated in FmocLL support this hypothesis, when encapsulated in FmocFF, the labeled HydA was accumulated directly on the carbon fibers, suggesting that the FmocFF fibrils directly interact with the carbon felt electrode (FIG. 5C). This difference can arise from FmocLL lacking the available hydrophobic motifs to achieve a strong adherence to the carbon fibers of the electrode. In addition, a physical mechanism could play a role in FmocFF retention ability as its fibrils appear to be physically entangled around the carbon fibers.

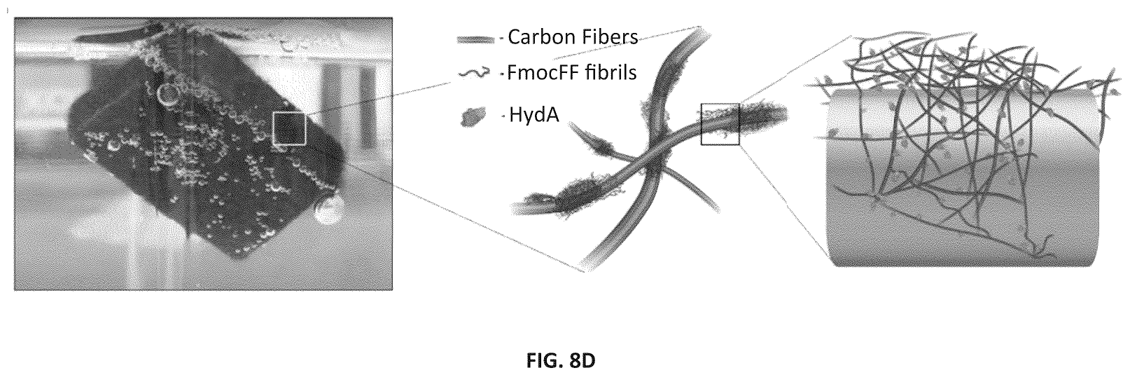

SEM measurements were used to evaluate the above assumptions. FIGS. 8A-C present scanning electron microscopy images of carbon felt electrode soaked with FmocFF hydrogel (5 mg/mL) at magnifications of ×40 (FIG. 8A), ×450 (FIG. 8B) and ×5500 (FIG. 8C). These high resolution scanning electron microscopy images indeed show hydrogel masses directly adhered onto the carbon fibers in FmocFF-soaked carbon felt electrode.

Taking together the interaction of FmocFF with HydA and with the carbon felt electrode, a mode of encapsulation is proposed in which FmocFF fibrils act as a glue, binding the protein to the electrode to achieve significantly improved retention, strong enough to resist the electrophoresis forces taking place under the tested experimental conditions, as schematically illustrated in FIG. 8D. This model can also explain why the 2 mg/mL suspensions show strong retention ability even as a liquid.

### Example 7

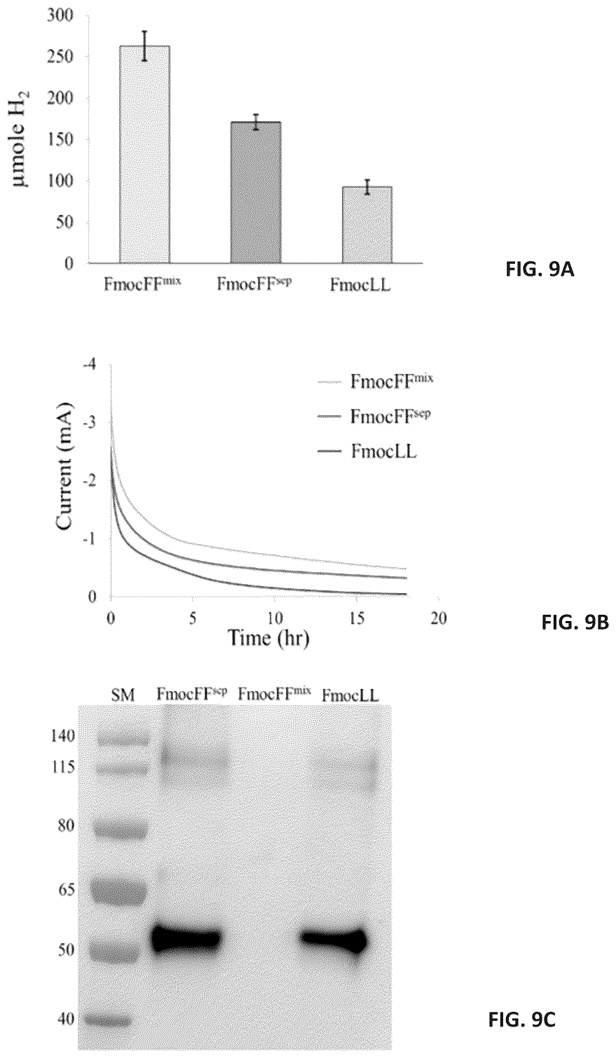

To further establish the model suggested in FIG. 8D, it was tested whether it is necessary to initiate the self-assembly of FmocFF fibrils in the presence of HydA and carbon felt electrode in order to achieve the observed efficient retention. The liquid state of self-assembled FmocFF at 2 mg/mL was utilized to prepare a fibril suspension prior to HydA introduction and carbon felt soaking. FmocFF was allowed to self-assemble overnight, and HydA was subsequently added to the suspension. The mixture was then incubated for 2 hours to allow the enzyme to interact with the assembled FmocFF fibrils before carbon felt soaking. The obtained electrodes were thereafter subjected to electrochemical measurements, and the obtained data is shown in FIGS. 9A-C.

FIG. 9A is a graph showing the H₂ accumulated from O·N. electrochemical activation of HydA enzyme, encapsulated in FmocFF in the presence of HydA and carbon felt electrode (FmocFFmix), in comparison to samples where Hyd A was added to FmocFF or to FmocLLmix (FmocFFsep). The results show that electrochemical H₂ production measurements were approximately 60% higher in samples where the fibrils were co-assembled in the presence of HydA and carbon felt (FmocFFmix), in comparison to samples where FmocFF was pre-assembled separately (FmocFFsep). FmocFFsep samples exhibited H₂ production 80% higher than FmocLL hydrogel samples.

FIG. 9B presents chronoamperometry graphs measuring current versus time of O·N. electrochemical assays of FmocFFmix, FmocFFsep, and FmocLL, which show that while the current of FmocFFsep samples decayed faster than FmocFFmix samples, the decay was still milder compared to that of FmocLL, further supporting the findings shown in FIG. 9A.

To assess whether these observations correspond to differences in retention ability, immunoblot assay was conducted as described above, as presented in FIG. 9C. While no detectable band was found in the electrolyte sample of FmocFFmix, a strong detectable HydA band was observed in both FmocLL and FmocFFsep electrolyte samples, implying enzyme loss.

The FmocFFsep band appears stronger than that of FmocLL, which contrasts with the higher current and H₂ production obtained from FmocFFsep. As the immunoblot assay was not designed to be quantitative, numerous factors can affect the visualized protein abundance (Pillai-Kastoori et al., Anal. Biochem., 2020, 593). Hence, the presence or absence of protein should be considered, rather than the strength of the band, which should be taken with care. Nonetheless, based on the higher current and H₂ production compared to FmocLL, it can be assumed at least a partial protein retention by the assembled fibrils of FmocFFsep.

These results suggest that while the presence of all the components upon assembly initiation is not an absolute requisite, it may augment enzyme retention. It is possible that initiating the assembly of FmocFF fibrils within the carbon felt results in better adherence of the fibrils to the carbon fibers, which increases the retention strength. Alternatively, the interaction between the FmocFF fibrils and the protein may be more efficient when the enzyme is present during fibril assembly. Nonetheless, the ability to introduce the components separately, while still preserving some retention ability, provides greater flexibility.

### Example 8

The generality of protein-fibrils interaction was evaluated by encapsulating two other Cy5-labeled redox enzymes; Ferredoxin-NADP Reductase (FNR); and Superoxide Dismutase (SOD) in 3 mg/mL FmocFF hydrogel.

In addition, the retention ability of FNR and SOD in the FmocFF hydrogel was assessed, when subjected to an electrochemical assay. For this purpose, 200 micrograms of either FNR or SOD together with 75 micrograms HydA in FmocFF hydrogels was encapsulated, which were soaked on carbon felt electrodes.

Following overnight electrochemical activation, an immunoblot assay was conducted to detect protein presence in the electrolyte.

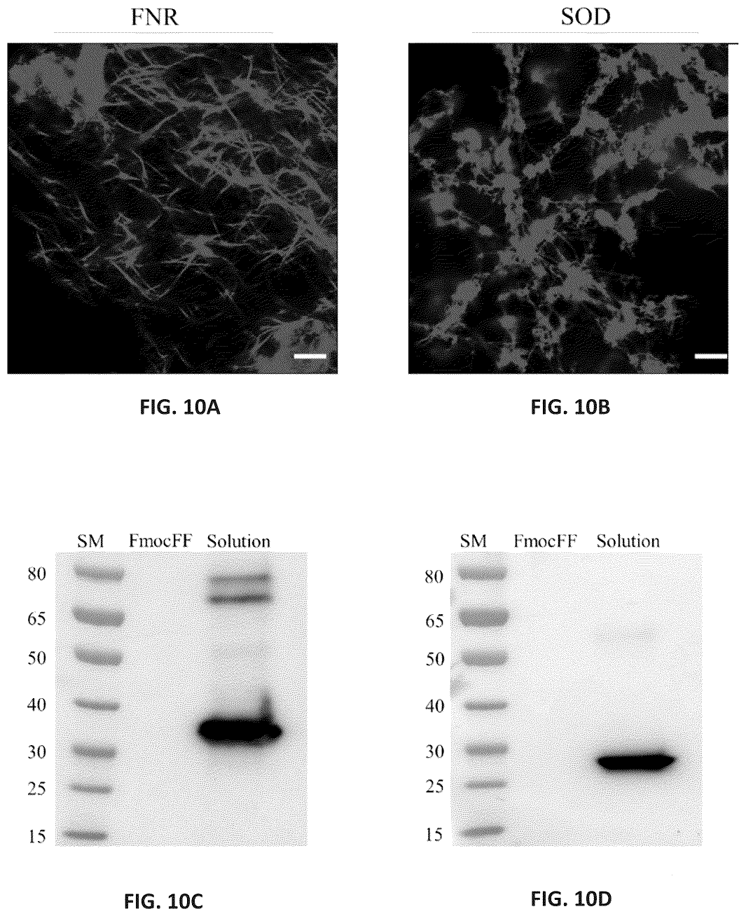

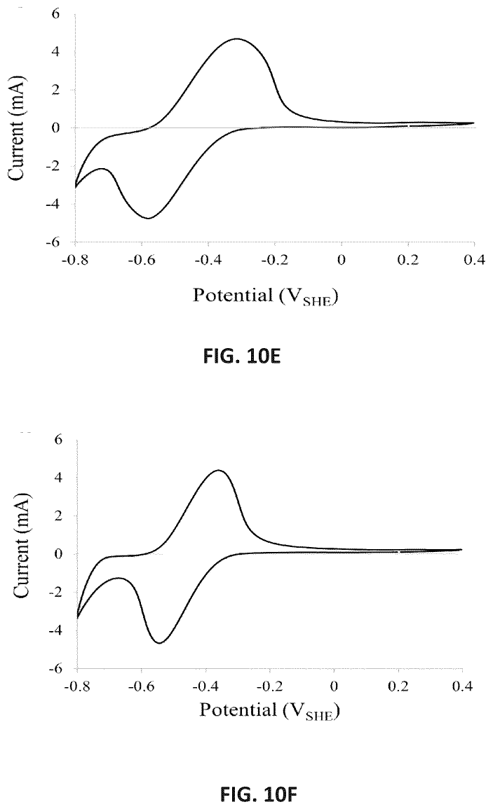

FIGS. 10A-B present confocal microscopy images of FmocFF hydrogel with Cy5-labeled FNR (FIG. 10A) and SOD (FIG. 10B). As can be observed similar to HydA-containing hydrogels, the encapsulation of stained FNR and SOD resulted in a fluorescent fibril matrix.

FIGS. 10E-F present cyclic voltammogram graphs of encapsulated FNR (FIG. 10E) and of encapsulated SOD (FIG. 10F). The electrochemical profile obtained by cyclic voltammetry of electrodes supplemented with MV and either FNR or SOD was similar to that obtained with MV-only loaded electrodes (not shown).

FIGS. 10C-D present immunoblots of FNR (FIG. 10C) and SOD (FIG. 10D) collected from electrolyte after O·N. electrochemical assays in FmocFF hydrogel or solution-soaked electrodes, and show that a specific protein band was observed for solution-soaked electrodes, demonstrating the inability of the carbon felt alone to retain the proteins. FmocFF-soaked electrodes successfully retained both HydA and FNR or SOD, with no detectable protein bands.

The similar structure and function observed for the three proteins examined suggest a general phenomenon of FmocFF fibril-protein interaction, which may be exploited for encapsulation of other proteins in a variety of electrochemical applications for energy, sensors, bionics, and electrochemical synthesis.

### Example 9

Whole E. coli bacteria expressing [FeFe] hydrogenase were immobilized on a carbon felt electrode and encapsulated in the hydrogel instead of pure protein, as follows: Electrode fabrication was performed in an anaerobic chamber (3.5% H₂ balance N₂, Coy Laboratories, USA). Tris-HCl pH 7.2 buffered solution was supplemented with 1 mM sodium dithionite, 2 mM MV and E. coli bacterial culture. Peptide hydrogelator powder was dissolved in DMSO to a concentration of 100 mg/mL. The two solutions were then mixed and immediately soaked on 2×1 cm carbon felt, followed by a 20-minutes gelation period. For FmocFF electrodes, a final concentration of 30% DMSO and 80 mM Tris-HCl pH 7.2 was used to allow sufficient soaking time due to rapid gelation in buffered solution. FmocLL electrodes were prepared at a final concentration of 10% DMSO, 80 mM Tris-HCl pH 7.2. For solution-soaked electrodes, only 80 mM Tris-HCl pH 7.2 buffer was used. Unless stated otherwise, all electrodes contained 3.2 O.D 600/mL E. coli bacterial culture, while 3 mg peptide hydrogelator was used in either FmocFF or FmocLL electrodes.

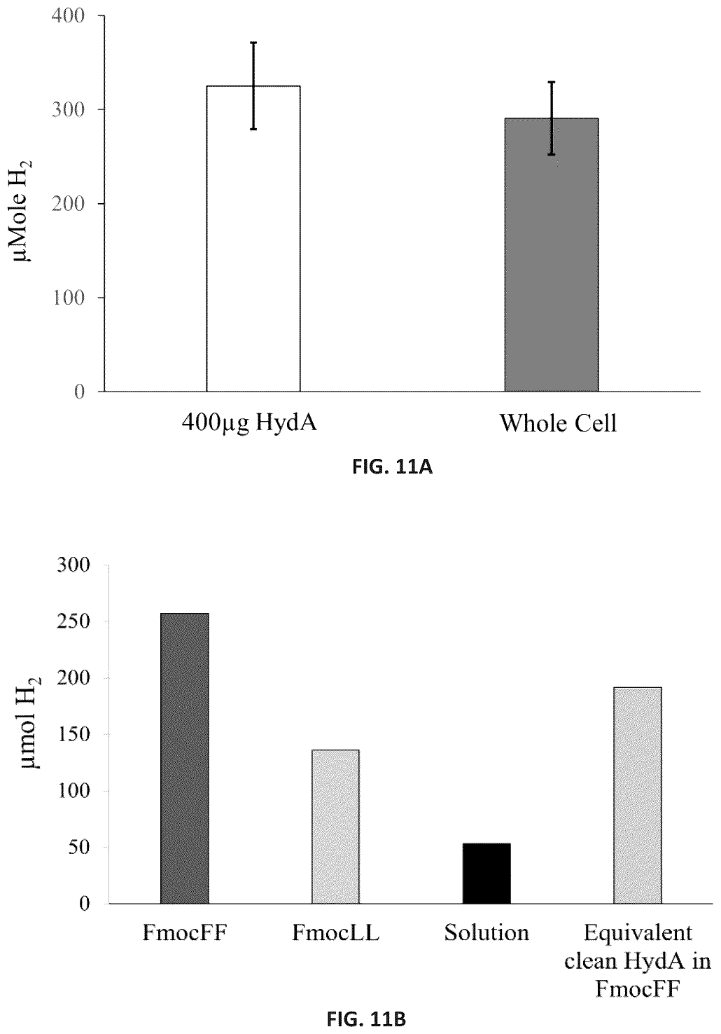

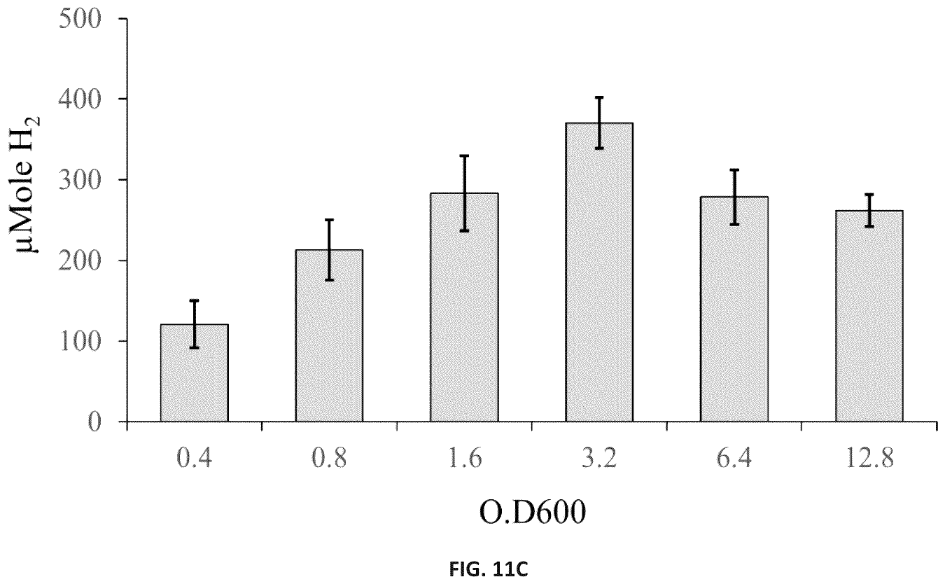

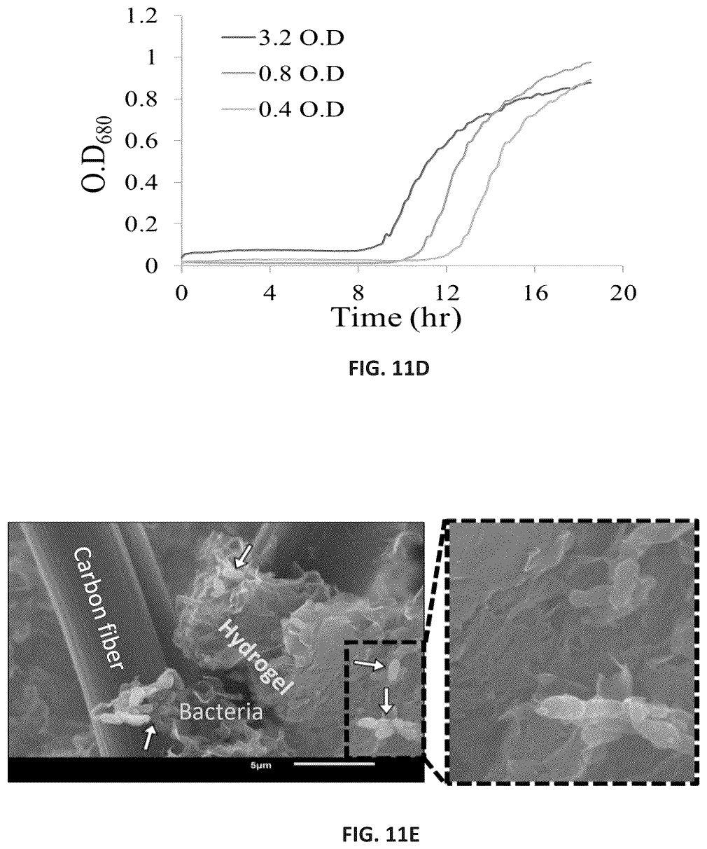

The obtained carbon felt electrodes were tested, and the obtained data is presented in FIGS. 11A-E.

FIG. 11A is a graph showing electrochemical H₂ production with 400 micrograms purified enzyme encapsulated in FmocFF hydrogel on carbon felt electrode, compared to encapsulated whole E. coli bacteria expressing [FeFe] hydrogenase, demonstrating a substantially similar performance of the tested electrodes.

FIG. 11B is a bar graph showing overnight electrochemical H₂ production of E. coli expressing HydA enzyme, encapsulated in FmocFF or FmocLL, or soaked from solution compared to purified clean HydA encapsulated in FmocFF, further showing a successful performance of the encapsulated bacteria electrode.

FIG. 11C is a graph showing the bacterial concentration dependent electrochemical H₂ production with encapsulated whole E. coli expressing [FeFe] hydrogenase, at OD levels of 0.4, 0.8, 1.6, 3.2, 6.4 and 12.8. As can be seen, when using MV as an electron mediator, higher bacterial concentrations lead to increased H₂ production, with optimal performance observed at a concentration of 3.2 O.D 600/mL.

FIG. 11D presents comparative plots showing the bacterial growth in LB medium at 37° C. post-electrode encapsulation at initial cell concentrations of 0.4, 0.8 and 3.2 cell O.D, over 20 hours. Bacteria were encapsulated on electrodes at varying concentrations (0.4, 0.8, and 3.2 O.D 600/mL), then submerged in 70 mL of LB medium and incubated at 37° C. for growth assessment using the Multi-Cultivator MC1000-OD (PSI). Optical density (O.D.) was measured at 10-minute intervals. The growth pattern reflects cell viability; higher viability of bacteria on the source electrode corresponds to early observed growth.

As can be seen, the growth rate correlated with concentration, with the earliest growth observed at a concentration of 3.2 O.D 600/mL. This indicates that the encapsulation process maintained bacterial viability, as evidenced by the growth observed.

SEM images of FmocFF hydrogel containing HydA-expressing E. coli bacteria attached to fibers on the carbon felt are presented in FIG. 11E, with white arrows indicating the position of the bacteria, and an inset demonstrating the bacterial entangled in the FmocFF fibril network. As can be seen, intact cells are bound to the carbon fibers, either individually or in groups, indicating that the FmocFF hydrogel successfully bound the bacteria to the carbon fibers while facilitating electron transfer.

Over all, these data suggest that encapsulating a whole bacteria can be successfully performed in a manner similar to that described above for the pure enzyme protein.

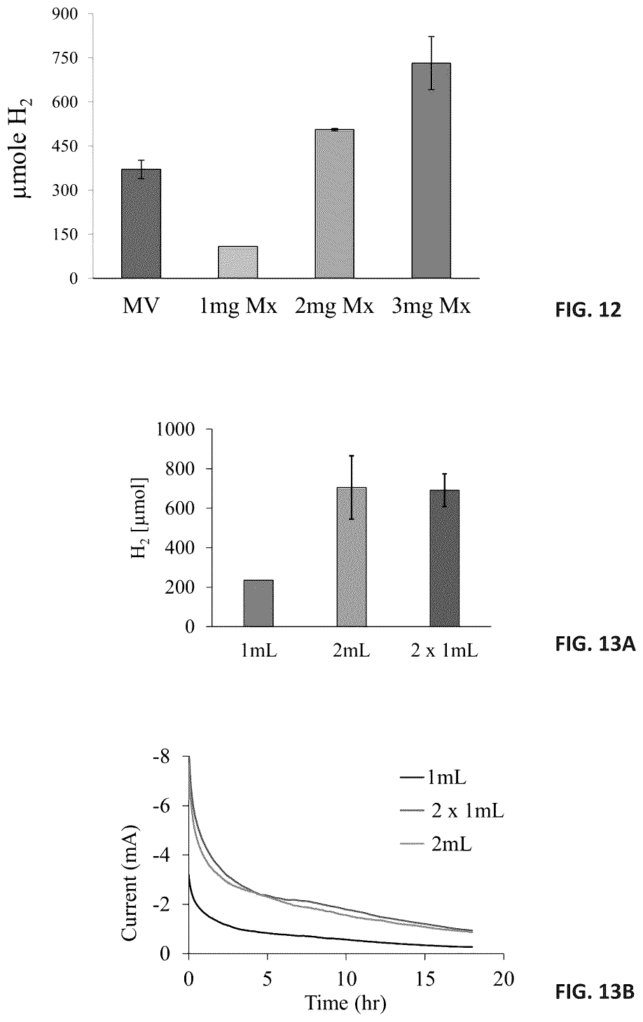

Similar electrodes were prepared with an exemplary electron transfer mediator of the MXene family, in accordance with the procedure described hereinabove. Titanium carbide Ti3C2Tx sheets were mixed with the buffer and bacteria before gelation with FmocFF. The MXene-infused gel were able to conduct electrons directly to the enzyme-bearing bacteria, allowing H₂ production. FIG. 12 presents the data obtained for the H₂ production performance of MV-mediated electrodes compared with electrodes employing MXene-based electron transfer mediator at various MXene concentrations, showing the efficient performance of the MXene as an electron transfer mediator.

### Example 10

A scaled-up electrochemical system was tested, by increasing the reaction volume and further increasing the electrode size or by tethering multiple electrodes (an array). The electrochemical set-up was assembled as described hereinabove, using electrodes featuring a volume of 1 mL, 2 mL, or 2 tethered 1 mL electrodes, and the H₂ O·N accumulation was determined, as described hereinabove. The obtained data is presented in FIGS. 13A-B. As can be seen, electrochemical H₂ production was increased in more than 2-folds in both systems, suggesting a successful scale-up.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A composition-of-matter comprising a fibrous electrode having associated therewith a self-assembled structure formed of a plurality of peptides each being of 2 to 6 amino acid residues in length, wherein in each of said peptides, at least one of said amino acid residues is an aromatic amino acid residue, and a biocatalyst, wherein said biocatalyst is associated with the self-assembled structure.

2. The composition-of-matter of claim 1, wherein said self-assembled structure is in a form of a hydrogel.

3. The composition-of-matter of claim 1, wherein at least one, or each, peptide in said plurality of peptides comprises an end-capping moiety.

4. The composition-of-matter of claim 3, wherein said end-capping moiety is aromatic.

5. The composition-of-matter of claim 1, wherein at least one, or each, of said peptides is a dipeptide.

6. The composition-of-matter of claim 5, wherein said dipeptide comprises diphenylalanine.

7. The composition-of-matter of claim 1, further comprising an electron transfer mediator.

8. The composition-of-matter of claim 1, wherein said biocatalyst is selected from an enzyme and an enzyme-producing microorganism.

9. The composition-of-matter of claim 1, wherein said biocatalyst catalyzes a redox reaction.

10. An electrochemical cell comprising, as a working electrode, the composition-of-matter of claim 1, and a power source.

11. The electrochemical cell of claim 10, further comprising an electrolyte, wherein said electrolyte comprises a substrate of said biocatalyst.

12. The electrochemical cell of claim 10, further comprising a reservoir configured for collecting a gas and/or liquid product formed by reducing or oxidizing said substrate in the presence of said biocatalyst, wherein when said product is a gas usable as fuel in a fuel cell, the electrochemical cell forms a part of a system that comprises said fuel cell.

13. An electrochemical system comprising the electrochemical cell of claim 10.

14. A method of electrically producing a gas and/or liquid formed in a redox reaction catalyzed by a biocatalyst, the method comprising contacting the composition-of-matter of claim 1, which comprises said biocatalyst, with a substrate of said biocatalyst, and applying potential to said electrode.

15. A method of performing a redox reaction of a substrate which is catalyzed by a biocatalyst, the method comprising contacting said substrate with the composition-of-matter of claim 1, which comprises said biocatalyst, and applying potential to said electrode, thereby reducing or oxidizing the substrate.

16. The method of claim 14, wherein said biocatalyst comprises or generates a hydrogenase, the method being for producing H₂; and/or wherein said biocatalyst comprises or generates a nitrogenase, the method being for producing ammonia and/or a salt thereof.

17. The method of claim 15, wherein said biocatalyst comprises or generates a hydrogenase, the method being for producing H₂; and/or wherein said biocatalyst comprises or generates a nitrogenase, the method being for producing ammonia and/or a salt thereof.

18. A fuel cell operated by the H₂ produced by the method of claim 16.

19. A process of preparing the composition-of-matter of claim 1, the method comprising contacting the electrode with a solution comprising said plurality of peptides and with a solution comprising said biocatalyst.

20. A fuel cell operated by the H₂ produced by the method of claim 17.
